(19) United States
(12) Patent Application Publication
(10) Pub. No.: US 2025/0011785 A1
(43) Pub. Date: Jan. 9, 2025

(54) Compositions and Methods for Modulating Single Stranded DNA Regions that Catalyze Enzymatic Reactions

(71) Applicant: The Trustees of the University of Pennsylvania, Philadelphia, PA (US)

(72) Inventors: James Eberwine, Philadelpia, PA (US); Sergei Vinogradov, Philadelphia, PA (US); Srinivasa Rao Allu, Philadelphia, PA (US); Jifen Li, Philadelphia, PA (US); Youtao Lu, Philadelphia, PA (US); Junhyong Kim, Philadelphia, PA (US)

(21) Appl. No.: 18/764,835

(22) Filed: Jul. 5, 2024

Related U.S. Application Data
(60) Provisional application No. 63/511,984, filed on Jul. 5, 2023.

(51) Int. Cl. C12N 15/113; C12N 15/10

(52) U.S. Cl. CPC C12N 15/113; C12N 15/1037; C12N 2310/127; C12N 2320/50

(57) ABSTRACT

Described are novel DNAzyme constructs with porphyrin metalation activity, methods of making and using them, as well as methods of identifying novel DNAzymes and methods of modulating the activity of DNAzymes.

### CROSS REFERENCE TO RELATED APPLICATIONS

This application claims priority to U.S. Provisional Application No. 63/511,984, filed Jul. 5, 2023, which is hereby incorporated by reference herein in its entirety.

### STATEMENT REGARDING FEDERALLY SPONSORED RESEARCH OR DEVELOPMENT

This invention was made with government support under AA028409 and HG010023 awarded by the National Institutes of Health. The government has certain rights in the invention.

### REFERENCE TO SEQUENCE LISTING SUBMITTED ELECTRONICALLY

This application contains a Sequence Listing, which is submitted electronically via EFS-Web as an XML Document formatted sequence listing with a file name “046483-6268-OOUS Sequence Listing.xml” having a creation date of Jul. 2, 2024, and having a size of 34,476 bytes. The sequence listing submitted via EFS-Web is part of the specification and is herein incorporated by reference in its entirety.

### BACKGROUND OF THE INVENTION

DNA 3D structure of eukaryotic cells is well recognized for regulating chromosome organization and RNA transcription. Apart from the B-form, double-stranded conformation (Kouzine et al., 2017, Cell Syst., 4, 344-356 e7), DNA also takes the non-B, single-stranded form (Sharma et al., 2011, J. Nucleic Acids., 724215). Stable ssDNA is thought to be involved in nucleosome localization, while transient ssDNA in replication, repair, recombination, and transcription. An example of transcriptionally related ssDNA is the “transcription bubble” (Bieberstein et al., 2012, Cell Rep., 2, 62-8; Barnes et al., 2015, Mol Cell. 59, 258-69). Specifically, the bubble moves along the length of the gene being transcribed, and in concert, long stretches of single-stranded areas longer than a kilobase form over the transcriptionally active chromatin (Kouzine et al., 2017, Cell Syst., 4, 344-356 e7; Bieberstein et al., 2012, Cell Rep., 2, 62-8; Zhou et al., 2015, Anal Biochem., 479, 48-50). Single-stranded DNA exists not only in genic regions but also in intergenic regions, including sites of DNA repair and replication, and these ssDNAs can be short or long lived and are reported to be regulated by a family of ssDNA binding proteins (Swamynathan et al., 1998, FASEB J., 12, 515-522). The amount of ssDNA in the genome is estimated to vary from ˜0.2% to 2.5%, depending upon the physiological state of the cell (Zhou et al., 2015, Anal Biochem., 479, 48-50).

Studying transcription in single cells in their natural microenvironment has been difficult, not only in the small amount of input material per cell, but also in the need to analyze the chromatin status before it is biochemically isolated. In efforts to understand brain cells' functional dynamics (especially the transcriptional potential) and to complement the other single-cell chromatin approaches, present experiments how a new method named CHEX-seq (Cromatin EXposed). It assumes that, as regions of transcribed open chromatin exist in single-stranded form, chemically crosslinking the chromatin can preserve the cytoarchitecture and allow detection and evaluation of the single-stranded regions in their natural context. CHEX-seq has been applied to explore ssDNA open chromatin in fixed dispersed neurons and astrocytes, and in situ localized single neurons preserving the cellular microenvironment. The CHEX-seq technology was used to identify single stranded DNA regions that can act catalytically.

There is a need in the art for compositions and methods for modulating single stranded DNA regions that catalyze enzymatic reactions. This invention satisfies this unmet need.

### SUMMARY OF THE INVENTION

The invention provides compositions and methods to modulate endogenous ssDNA sequences having enzymatic or catalytic activity as well as methods of use thereof for increasing the enzymatic or catalytic activity of an endogenous ssDNA sequence. Also provided are assay systems for identifying novel endogenous ssDNA sequences having enzymatic or catalytic activity.

In one embodiment, the invention relates to a composition comprising a modulator of a catalytic single stranded DNA molecule (DNAzyme).

In one embodiment, the modulator comprises an inhibitor of the DNAzyme. In one embodiment, the inhibitor disrupts or destabilizes the conformation of the DNAzyme.

In one embodiment, the modulator comprises an activator of the DNAzyme. In one embodiment, the activator generates or stabilizes a specific structure within the ssDNA region to increase the activity of the DNAzyme.

In one embodiment, the activator comprises an oligonucleotide comprising at least one region that binds to an internal ssDNA region of the DNAzyme. In one embodiment, the oligonucleotide is a DNA oligonucleotide or an RNA oligonucleotide. In one embodiment, the oligonucleotide comprises at least one modified base.

In one embodiment, the activator comprises an oligonucleotide comprising at least one region that binds to an internal ssDNA region of the DNAzyme, wherein binding of the oligonucleotide generates or stabilizes a specific structure within the ssDNA region.

In one embodiment, the structure is a loop structure comprising the catalytically active site of the DNAzyme. In one embodiment, the DNAzyme is TATDN2P1short (SEQ ID NO:28), TATDN2P1long (SEQ ID NO:27) or RPL7AP61 (SEQ ID NO:32).

In one embodiment, the modulator comprises a clamping oligonucleotide selected from the group consisting of opTATDN2Planneal-short-loop (SEQ ID NO: 29) and opTATDN2Planneal-long-loop (SEQ ID NO:30).

In one embodiment, the invention relates to a method of modulating the level or activity of a DNAzyme, the method comprising contacting the DNAzyme with a composition comprising a modulator of a catalytic single stranded DNA molecule (DNAzyme).

In one embodiment, the DNAzyme is a plant DNAzyme, a mammalian DNAzyme, or a synthetic DNAzyme.

In one embodiment, the DNAzyme is TATDN2P1short (SEQ ID NO:28), TATDN2P1long (SEQ ID NO:27) or RPL7AP61 (SEQ ID NO:32).

In one embodiment, the invention relates to a composition comprising an isolated nucleic acid molecule comprising a porphyrin metalation DNAzyme, wherein the DNAzyme is TATDN2P1short (SEQ ID NO:28), TATDN2P1long (SEQ ID NO:27) or RPL7AP61 (SEQ ID NO:32).

In one embodiment, the invention relates to a method for modulating porphyrin metalation in a sample, the method comprising contacting the sample with a composition comprising an isolated nucleic acid molecule comprising a porphyrin metalation DNAzyme, wherein the DNAzyme is TATDN2P1short (SEQ ID NO:28), TATDN2P1long (SEQ ID NO:27) or RPL7AP61 (SEQ ID NO:32).

In one embodiment, the method comprises contacting the sample with a DNAzyme of TATDN2P1short (SEQ ID NO:28), TATDN2P1long (SEQ ID NO:27) or RPL7AP61 (SEQ ID NO:32).

In one embodiment, the method comprises contacting the sample with a complex comprising a) a DNAzyme; and b) a clamping oligonucleotide. In one embodiment, the DNAzyme is TATDN2P1short (SEQ ID NO:28), TATDN2P1long (SEQ ID NO:27) or RPL7AP61 (SEQ ID NO:32). In one embodiment, the clamping oligonucleotide is opTATDN2Planneal-short-loop (SEQ ID NO: 29) or opTATDN2Planneal-long-loop (SEQ ID NO:30).

In one embodiment, the invention relates to a method of treating a disease or disorder in a subject in need thereof, the method comprising administering a composition comprising a modulator of a catalytic single stranded DNA molecule (DNAzyme) to modulate the activity of a DNAzyme in the subject, wherein the subject has a disease or disorder that would benefit from modulation of the DNAzyme.

In one embodiment, the disease or disorder is cancer, a neurodegenerative disease, a mitochondrial disease, a disease associated with a DNA repair defect, a disease associated with telomere shortening, a bacterial disease or a viral disease.

In one embodiment, the invention relates to a method of identifying a DNAzyme, the method comprising
a) performing a CHEX-seq (CHromatin EXposed) assay to identify single-stranded open chromatin regions in a genomic or mitochondrial DNA sample,
b) identifying at least one catalytic core sequence of a DNAzyme having a desired activity,
c) aligning the catalytic core sequence of the DNAzyme having the desired activity with the single-stranded open chromatin regions in a genomic or mitochondrial DNA sample,
d) identifying homologous ssDNA loci in the sample as potential DNAzymes, and
e) confirming the DNAzyme activity of the homologous ssDNA loci.

In one embodiment, the method further comprises a step of synthesizing a modulator of the DNAzyme.

In one embodiment, the invention relates to a method of identifying a catalytic nucleic acid molecule, the method comprising performing a de novo gDNAzyme sequences (DEAS) assay comprising the steps of:
a) isolating endogenous single stranded DNA from at least one genomic or mitochondrial DNA sample;
b) cloning at least a portion of the isolated ssDNA into a single-strand phage to generate a library of phage-cloned ssDNA;
c) contacting the phage-cloned ssDNA library with at least one test nucleic acid molecule under conditions that allow for the generation of at least one modified nucleic acid product by a catalytic nucleic acid molecule;
d) collecting and sequencing at least one modified nucleic acid product to identify the target cleavage sequence of a catalytic nucleic acid molecule, and
e) contacting at least a portion of the sample with a bait nucleic acid molecule comprising the target cleavage sequence of the catalytic nucleic acid molecule to capture the catalytic nucleic acid molecule.

In one embodiment, at least one modified nucleic acid product is a DNA cleavage product, an RNA cleavage product, a DNA ligation product, RNA ligation product, DNA phosphorylation product or an RNA phosphorylation product.

In one embodiment, the method further comprises a step of sequencing the captured DNAzyme.

In one embodiment, the method further comprises a step of contacting the ssDNA phage library with ssDNA endonuclease prior to step c).

In one embodiment, at least one test nucleic acid molecule of step c) is a genonic DNA molecule.

In one embodiment, at least one test nucleic acid molecule of step c) is an RNA molecule.

In one embodiment, the method is for identifying a catalytic nucleic acid molecule having DNA cleavage activity, RNA cleavage activity, DNA ligation activity, RNA ligation activity, DNA phosphorylation activity, or RNA phosphorylation activity.

In one embodiment, the catalytic nucleic acid molecule is a gDNAzyme or an mtDNAzyme.

In one embodiment, the sample comprises a neuron, and the endogenous single stranded DNA is extracted from isolated intact neuronal nuclei.

In one embodiment, the sample comprises fixed dispersed primary cell cultures or fixed tissue sections. In some embodiments, the fixed tissue sections are brain tissue sections.

In one embodiment, the invention relates to a catalytic DNA molecule isolated using a de novo gDNAzyme sequences (DEAS) assay.

In one embodiment, the catalytic DNA molecule is a gDNAzyme or an mtDNAzyme.

In one embodiment, the catalytic nucleic acid molecule has DNA cleavage activity, RNA cleavage activity, DNA ligation activity, RNA ligation activity, DNA phosphorylation activity or RNA phosphorylation activity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1G depict representative results from experiments demonstrating CHEX-seq experimental design, K562 priming location characterization, and overlap with K562 transcriptome. FIG. 1A depicts a schematic of CHEX-seq assay. FIG. 1B depicts representative photoactivation at a specific site in the nucleus of K562. FIG. 1C shows the representative statistics of CHEX-seq priming sites with respect to genomic regions. FIG. 1D depicts representative TSS proximal (+/−5 kb) coverage of K562 samples (all positive non-outlier samples aggregated, top 1%-tile genes shown). FIG. 1E shows the representative Z-scored coverage at TSS (+/−5 kb) and CDS (+/−3 kb) proximity at single-cell level; Single: single-cell samples, Bulk: multi-cell samples, All: aggregates of Single and Bulk. FIG. 1F depicts the overlap between CHEX-seq primed genes (extended gene body>0) and RNA-seq highly expressed genes (counts>median). FIG. 1G depicts the representative GO functional enrichment results (top 20 significant) of the CHEX-RNA overlapping genes (F, left), x-axis, −log 10(Benjamini-Hochberg adjusted p-value).

FIG. 2A through FIG. 2C depict representative results from experiments demonstrating genomic comparisons of CHEX-seq with other open chromatin assays (ATAC-, DNase-, FAIRE-seq), transcriptome (GRO-seq), and epigenomes (RRBS DNA methylation, histone modifications, super enhancer [SE]). FIG. 2A depicts the UCSC Genome Browser track view comparing the coverage of CHEX- (purple) against ATAC- (red), DNase- (blue) and FAIRE-seq (green) at locus OTUD5. Below four assays is the GeneCards TSS track. The last four tracks are transcriptome and three histone marks. Dashed-line boxes highlight two loci shared by all four open chromatin assays. FIG. 2B shows the representative hierarchical clustering of open chromatin assays, transcriptome, and epigenomes at 5 kb (left) or 50 kb (right) resolution, using binarized coverage and Jaccard distance. FIG. 2C depicts the representative hierarchical clustering of CHEX-, ATAC-, DNase- and FAIRE-seq by the similarity with an extended set of 284 K562 epigenomes. Color indicates quantile normalized fold of enrichment given a particular assay (0 means the lowest enrichment and 1 means the highest enrichment).

FIG. 3A through FIG. 3E depict representative results from experiments demonstrating expression levels of genes with TSS priming stratified by the distance to TSS (maximum 2.5 kb to either direction). FIG. 3A depicts the representative bulk K562 RNA-seq. FIG. 3B depicts the representative bulk K562 GRO-seq. FIG. 3C depicts the representative K562 scRNA-seq, single cells aggregated. Y-axis, gene expressions (capped at 90%-tile); x-axis, distance (bp) to TSS from CHEX-seq priming sites. FIG. 3D and FIG. 3E depict the representative CHEX-seq unique property: detecting open chromatin's strandedness. FIG. 3D shows a schematic showing the hypothesis that CHEX-seq reads should have opposite strandedness than sense-strand mRNA transcripts; FIG. 3E shows representative results testing the hypothesis in (FIG. 3D). X-axis, sub-genic regions; y-axis, ratio of the number of antisense-stranded over sense-stranded CHEX-seq reads, samples with less than 5 counts discarded; significance by Wilcoxon rank-sum test (two-sided).

FIG. 4A through FIG. 4D depict representative results from experiments demonstrating TPA-induced dynamics in distribution of ssDNA throughout the genome. FIG. 4A depicts the representative changes in the distribution of CHEX-seq priming location (relative to TSS) upon TPA perturbation. Y-axis: cumulative empirical distribution of CHEX-seq priming sites with the distance to TSS; x-axis: distance to TSS (bp). FIG. 4B depicts representative differentially primed genes in acute response to TPA treatment. The heatmap color indicates the priming counts in log 2. FIG. 4C depicts the representative genes losing (top) or gaining (bottom) TSS single-strandedness in concert to the TSS enrichment changes. The heatmap color indicates the priming counts in log 2. FIG. 4D shows the top 50 GO (Molecular Function) terms enriched in the genes losing TSS single-strandedness.

FIG. 5A through FIG. 5F depict representative results from experiments demonstrating CHEX-seq applied to mouse neuronal samples (fixed tissue section and dispersed culture). FIG. 5A depicts the representative schematic of CHEX-seq applied to mouse brain tissue section using cell-specific markers. FIG. 5B depicts the representative photoactivation of CHEX-seq probes (red) at single neuronal cell stained with the marker MAP2 (green) in mouse brain section. FIG. 5C shows the representative TSS (±5 kb) (top) and CpG island flanking (±500 bp) (bottom) CHEX-seq coverage in mouse brain section. FIG. 5D shows the representative correlation between CHEX-seq intronic priming frequency and transcriptional activity (RNA-seq intronic counts) in mouse brain section. FIG. 5E and FIG. 5F depicts the representative correlation between CHEX-seq intronic priming frequency and transcriptional variability (RNA-seq intronic counts) in mouse brain section.

FIG. 6A and FIG. 6B depict representative results from experiments demonstrating CHEX-seq unveiling the mitochondrial ssDNA in mouse brain. FIG. 6A depicts the representative mitochondrial single-stranded hotspots found in mouse astrocytes and neurons in primary cultures, and mouse neuron and interneuron in brain sections. X-axis: coordinate (bp) of the mitochondrial genome; y-axis: per-base priming counts (z-scored). Green dashed lines highlight where z-score equals to 1 or 2. The red font indicates the heavy-strand genes (upper gray boxes), and the blue font indicates the light-strand genes (lower gray boxes); the dark-light alternating font color corresponds to the shade of the gray boxes, to make adjacent genes more discernible. FIG. 6B depicts the representative CHEX-seq priming density (y-axis) inside (red) or outside (green) the D-loop (top) and the CHEX-seq priming bias (bottom) as measured as the ratio of the heavy-strand counts to the light-strand counts (y-axis).

FIG. 7A through FIG. 7E depict representative results from experiments demonstrating that genomic ssDNA regions can catalyze porphyrin metalation in vitro. FIG. 7A depicts representative results showing insertion of Zn2+ into mesoporphyrin IX (mPIX) catalyzed by candidate ssDNA regions of human or mouse gDNA. The plots represent the time course of the absorbance at 410 nm corresponding to the characteristic peak (Soret band) of Zn-mPIX (normalized by the porphyrin concentration). Spectra were captured every 30 min. Solid line: Zn+++Pb++, dashed line: Zn++ only. Red: TATDN2P1 (A,B), green: RPL7AP61 (C,D), blue: Bmpr1 (E,F); native gDNA (G), denatured gDNA (H), denatured HaeIII cut gDNA (I). FIG. 7B depicts the representative slopes of catalytic activity curves. Data are presented as mean±SEM. ns, not significant. *, p<0.05. ***, p<0.001. FIG. 7C depicts the representative comparison of catalytic activity slopes for gDNAzyme w/and w/out Pb2+ cofactor, and mouse genomic DNA. SEM***, p<0.001. FIG. 7D depicts the representative catalytic activity of TATDN2)1 DNAzyme in various conformations. Green: single-stranded short sequence, Orange: single-stranded long sequence, Red: long sequence constrained to form a short loop, Blue: long sequence constrained to form a long loop. FIG. 7E shows representative mouse cortical neurons in primary cell culture interrogated with FRET-FISH. Single-stranded DNA areas that contain sequences capable of acting catalytically from the RPL and TATDN2P1genes are identified with red dots on the periphery of neuronal nucleus. Calibration bar=20 μm.

FIG. 8 depicts representative chemical synthesis workflow of the CHEX-seq probe (BC1 for example. For the complete barcode/primer list see Table 1).

FIG. 9 depicts the representative single-stranded open chromatin model and the CHEX-seq protocol. Dashed-line boxes denote the two sequences that define the CHEX-seq barcode/primer quality. The bold font denotes the barcode/primer identifier (BC1 for example. For the complete barcode/primer list see Table 1).

FIG. 10 describes the CHEX-seq barcode/primer quality classes and subclasses under A, B and C.

FIG. 11A through FIG. 11E depicts the representative results from experiments demonstrating the quality assessment of CHEX-seq priming counts and coverage in K562 positive and control samples. FIG. 11A depicts the total number of priming sites. FIG. 11B depicts the total number of priming genes. FIG. 11C through FIG. 11E depict the representative priming coverage enrichment around TSS+/−5 kb region in (FIG. 11C) positive samples, (FIG. 11D) negative controls and (FIG. 11E) mung bean digested controls.

FIG. 12 depicts the representative results from experiments demonstrating the comparison between CHEX-seq and ATAC-seq in TSS coverage profiles using reads extended to the same size (2 kb) for CHEX-seq and ATAC-seq. Both assays have mitochondrial and chrY reads removed. The coverage of top 1% (n=581) high-coverage genes across 500 bins within TSS±5 kb is shown in the heatmap (bottom), while gene-averaged coverage is shown as the curve (top).

FIG. 13A through FIG. 13C depicts the representative results from experiments demonstrating CHEX-epigenome association analysis. FIG. 13A shows the representative hierarchical clustering of CHEX-, ATAC-, DNase- and FAIRE-seq by the similarity with the extended set of 284 K562 epigenomes. CHEX-seq reads have been filtered with more stringent criteria than FIG. 2C. Color indicates quantile normalized fold of enrichment per assay (0 means the lowest enrichment and 1 means the highest enrichment). NIM: Narrow Histone Marks. BHM: Broad Histone Marks. FIG. 13B depicts CHEX-seq association with non-B-form DNA, ATAC-, DNase- and FAIRE-seq in human brain; color indicates the fold of enrichment in log 2. FIG. 13C depicts CHEX-seq association with non-B-form DNA, ATAC-, DNase-seq and ENCODE histone marks in mouse brain (8 weeks adult); color indicates the fold of enrichment in log 2.

FIG. 14A through FIG. 14F depict the representative results from experiments demonstrating FISH validation of single-stranded, intergenic loci in K562. FIG. 14A depicts UCSC Genome Browser visualization of read coverage from CHEX-, ATAC-, DNase-, and FAIRE-seq; the lower panel shows GeneCards TSS, GeneHancer enhancers and promoters as well as chromatin interactions in between. FIG. 14B through FIG. 14F depicts representative DAPI and FISH images of the K562 cells showing three annealing sites at the locus in (FIG. 14A), possibly due to the trisomy chromosomes in K562; (FIG. 14C-FIG. 14F) Additional FISH validation for CHEX-seq identified loci at (FIG. 14C) chr4:1466001-1468000, (FIG. 14D) chr8:116514001-116516000, (FIG. 14E) chr11:123002001-123004000, (FIG. 14F) chrX:55182001-55184000.

FIG. 15A through FIG. 15F depict representative results from experiments demonstrating CHEX-seq coverage profiles in gene-body, 5′ UTR, TSS and CpG Island flanking regions in mouse neuron and interneuron tissue section samples. FIG. 15A depicts gene-body±5 kb flanking in mouse neuron tissue section. FIG. 15B depicts 5′ UTR±500 bp flanking in mouse neuron tissue section. FIG. 15C depicts TSS±5 kb flanking in mouse interneuron tissue section. FIG. 15D depicts CpG island ±500 bp flanking in mouse interneuron tissue section. FIG. 15E gene-body±5 kb flanking in mouse interneuron tissue section. FIG. 15F depicts 5′ UTR 500 bp flanking in mouse interneuron tissue section.

FIG. 16A through FIG. 16G depict representative results from experiments demonstrating the correlation between CHEX-seq priming distance to the TSS (x-axis) and RNA-seq gene expression (y-axis). (FIG. 16A) Human astrocyte culture; (FIG. 16B) Human neuron culture; (FIG. 16C) Human interneuron culture; (FIG. 16D) Mouse astrocyte culture; (FIG. 16E) Mouse neuron culture; (FIG. 16F) Mouse neuron tissue section; (FIG. 16G) Mouse interneuron tissue section.

FIG. 17A through FIG. 17C depict representative results from experiments demonstrating the overlap between CHEX-seq and transcriptome from the corresponding cell type in various sub-genic regions. FIG. 17A depicts the representative schematic defining the various sub-genic regions. TES: transcription end site. FIG. 17B depicts the representative odds ratio (log 2) for the overlap between CHEX-seq primed genes (cells aggregated, binarized at zero) and highly expressed genes (binarized at the median) of matched cell types (except for human interneuron where human neuron RNA-seq is used) in sub-genic regions ([G]eneExt, [P]romoter, [E]xon, [I]ntron, [D]ownstream). FIG. 17C depicts the representative odds ratio (log 2) for the overlap between CHEX-seq primed genes (binarized at zero) and highly expressed nascent RNAs (GRO-seq binarized at the median) in K562, showing single-cell, bulk and aggregated data.

FIG. 18A and FIG. 18B depict representative results from experiments demonstrating mitochondrial priming patterns in human and mouse. FIG. 18A depicts the representative single-cell and bulk samples' priming status (binarized: green if primed) for 37 human mitochondrial encoded genes in K562 control and TPA treated samples. FIG. 18B depicts the representative single-cell and population samples' priming status (binarized: green if primed) for 37 mouse mitochondrial encoded genes in mouse brain samples.

FIG. 19A through FIG. 19H depict representative results from experiments demonstrating single-base strand specific priming counts in human and mouse mitochondrial genome. (FIG. 19A) K562; (FIG. 19B) Human astrocyte culture; (FIG. 19C) Human neurons in culture; (FIG. 19D) Human interneurons in culture; (FIG. 19E) Mouse astrocytes in culture; (FIG. 19F) Mouse neurons in culture; (FIG. 19G) Mouse neuron in brain section; (FIG. 19H) Mouse interneuron in brain section. X-axis: coordinate (bp) of the mitochondrial genome; y-axis: per-base priming counts in the plus (y>0) and minus (y<0) strand of the mitochondrial genome. Green dashed boxes highlight regions sharing strand-specific priming across mouse brain samples. The dark-light alternating font color corresponds to the shade of the gray boxes, to make adjacent genes more discernible.

FIG. 20 depicts a DEAS strategy for cloning and functionalization of endogenous ss gDNA. This approach uses single-stranded phage as cloning and activity vectors.

FIG. 21 depicts representative results from experiments demonstrating DEAS isolation of ssDNA and potential gDNAzymes from fixed tissue sections. Low concentrations of Mung Bean nuclease are added to fixed tissue sections after deparaffinization (Left). The time of treatment was optimized with 3 min providing a good distribution of single stranded DNA which was liberated from the cellular nuclear and mitochondrial DNA (Right).

FIG. 22 depicts representative results from DNA-directed catalytic activity of cloned ssDNA isolated from fixed cells. Phage-cloned ssDNA isolated from a fixed brain tissue section was incubated with double-stranded genomic DNA to assess whether catalytic activity can be detected. The reaction conditions need to be optimized with NaCl concentration being tested in these data. After a 2 hour incubation, the reactions were run on a bioanalyzed to check for DNA cleavage. Lane 1 is the marker lane. Lanes 2-4 are complete reactions with ssDNA library, substrate double-stranded gDNA testing NaCl dependence. Uncut gDNA is shown in the lanes 5-6 and the phage library is shown in the 7^{th} lane.

FIG. 23A through FIG. 23C depict representative results from experiments demonstrating mitochondrial Cox1 RNA cleaving DNAzyme. FIG. 23A) CHEX-seq ss mtDNA reads map across the genome with a region concentrated in the Cox1 genes (red rectangle). FIG. 23B) in vitro testing of the enzymatic activity of Cox1 positioned DNAzyme shows that it cuts Cox1 mRNA at the predicted site (red arrows) (lanes—1-size markers, 2—uncut RNA, 3—one hour cutting, 4—three hour cutting). FIG. 23C) Cox1 primer extension from two independent mouse brain total RNA. Red arrow is primer binding site. The termination site of primer extended cDNAs coincident with the 5′end of the predicted cleavage site of the endogenous Cox1 mtDNAzyme (green arrow).

### DETAILED DESCRIPTION

In some embodiments, the invention provides compositions and method to modulate endogenous ssDNA sequences having enzymatic or catalytic activity. In some embodiments, the compositions comprise modulators for increasing the enzymatic or catalytic activity of an endogenous ssDNA sequence. In some embodiments, the compositions comprise modulators for decreasing the enzymatic or catalytic activity of an endogenous ssDNA sequence. In some embodiments, the endogenous ssDNA sequence is a genomic ssDNA sequence having enzymatic or catalytic activity (gDNAzyme). In some embodiments, the endogenous ssDNA sequence is a mitochondrial ssDNA sequence having enzymatic or catalytic activity (mtDNAzyme).

Methods of modulating endogenous ssDNA sequences having enzymatic or catalytic activity find use in multiple fields including, but not limited to, agricultural applications, environmental applications, and medical applications. In various embodiments, the methods can be used for modulating a gDNAzyme or mtDNAzyme in a plant cell, a bacterial cell, a virus, or an animal cell. In some embodiments, the methods can be used for modulating cell division, or for the treatment of diseases or disorders including cancer, neurodegenerative disease, mitochondrial disease, diseases associated with DNA repair defects, and diseases associated with telomere shortening,

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or +0.1% from the specified value, as such variations are appropriate to perform the disclosed methods.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding such regulatory polypeptides, that may optionally include intron sequences that are derived from chromosomal DNA. The term “intron” refers to a DNA sequence present in a given gene that is not translated into protein and is generally found between exons. As used herein, the term “transfection” means the introduction of a nucleic acid, e.g., an expression vector, into a recipient cell by nucleic acid-mediated gene transfer.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

The term “nucleic acid enzyme” refers to a nucleic acid molecule that catalyzes a chemical reaction. The nucleic acid enzyme may be covalently linked with one or more other molecules yet remain a nucleic acid enzyme. Examples of other molecules include dyes, quenchers, proteins, and solid supports. The nucleic acid enzyme may be entirely made up of ribonucleotides, deoxyribonucleotides, or a combination of ribo- and deoxyribonucleotides.

The term “catalytic nucleic acid”, “catalytic DNA”, “deoxyribozyme”, “DNA enzyme” or “DNAzyme” as used herein may refer to a nucleic acid molecule or oligonucleotide sequence that can catalyze or initiate a reaction. DNAzymes may be single-stranded DNA, and may include RNA, modified nucleotides and/or nucleotide derivatives. In some embodiments, the DNAzyme is “RNA-cleaving” and catalyzes the cleavage of a particular substrate, for example a nucleic acid sequence comprising one or more ribonucleotides, at a defined cleavage site. In some embodiments, the substrate is a target nucleic acid in a sample. In some embodiments, the DNAzyme cleaves a single ribonucleotide linkage. In some embodiments, the single ribonucleotide linkage is in a nucleic acid sequence wherein the remaining nucleotides are ribonucleotides. In some embodiments, the single ribonucleotide linkage is in a nucleic acid sequence wherein the remaining nucleotides are deoxyribonucleotides. In some embodiments, the DNAzyme cleaves a nucleic acid sequence at a single ribonucleotide linkage thereby producing a nucleic acid cleavage fragment.

The term “hybridize” refers to the annealing of one single-stranded nucleic acid molecule to another nucleic acid molecule based on sequence complementarity. The propensity for hybridization between nucleic acids depends on the temperature and ionic strength of their milieu, the length of the nucleic acids and the degree of complementarity. The effect of these parameters on hybridization is well known in the art.

The term “inhibit” means to slow, stop or otherwise impede.

The term “treatment or treating” as used herein may refer to an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “virus” as used herein may refer to an organism of simple structure, composed of proteins and nucleic acids, and capable of reproducing only within specific living cells, using its metabolism. In some embodiments, the virus is an enveloped virus, a non-enveloped virus, a DNA virus, a single-stranded RNA virus and/or a double-stranded RNA virus. Non-limiting examples of virus include rhinovirus, myxovirus (including influenza virus), paramyxovirus, coronavirus such as SARS-CoV-2, noro virus, rotavirus, herpes simplex virus, pox virus (including variola virus), reovirus, adenovirus, enterovirus, encephalomyocarditis virus, cytomegalovirus, varicella zoster virus, rabies lyssavirus and retrovirus (including HIV).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Nucleic acid agents able to act on specific mRNAs to silence the expression of target genes at transcript- or allele-specific levels have been exploited by many labs around the world for decades. Among them, DNAzymes have been extensively studied with promising results for use as therapeutic agents. The basic structure of DNAzymes comprises a catalytic domain flanked by two substrate binding arms with their sequences complementary to targeted mRNA sequence. DNAzymes have shown different reaction rates toward different nucleotide composition at mRNA cleavage site. Besides, unlike siRNAs which require Dicer protein to form RNA-induced silencing complex (RISC) for mRNA cleavage, divalent metal ions such as Mg2+ or Ca2+, which are abundant in cell cytosol, are sufficient for catalyzing DNAzyme function. Combining these factors together, DNAzymes are cheap, stable, and easily manipulated nucleic acid agents with highly efficient mRNA cleavage activity and low non-specific toxicity in cancer therapy.

### Methods for Identification of Novel DNAzymes

In some embodiments, the invention provides a method for identifying novel catalytic nucleic acid molecules in a cell or organism of interest. In some embodiments, the catalytic nucleic acid molecule comprises a genomic DNAzyme (gDNAzyme) or a mitochondrial DNAzyme (mtDNAzyme). The catalytic nucleic acid molecules may have nuclease activity on DNA or RNA molecules.

In some embodiments, the method comprises performing a CHEX-seq (CHromatin EXposed) assay to identify single-stranded open chromatin regions in a genomic or mitochondrial DNA sample, identifying at least one catalytic core sequence of a DNAzyme having a desired activity, aligning the catalytic core sequence of the DNAzyme having the desired activity with the single-stranded open chromatin regions in a genomic or mitochondrial DNA sample, and identifying homologous ssDNA loci in the sample. In some embodiments, the invention further comprises confirming the DNAzyme activity of the homologous ssDNA loci. The CHEX-seq assay is described in International Patent Application Publication WO2020146312A1, which is incorporated herein in its entirety.

In some embodiments, the method comprises a de novo gDNAzyme sequences (DEAS) assay for identifying a catalytic nucleic acid molecule.

In some embodiments, the method comprises the steps of: a) isolating endogenous single stranded DNA from at least one genomic or mitochondrial DNA sample; b) cloning a portion of the isolated ssDNA into a single-strand phage to generate a library of phage-cloned ssDNA; c) contacting the phage-cloned ssDNA library with test nucleic acid molecules under conditions that allow for the generation of DNA or RNA cleavage products by a catalytic nucleic acid molecule; d) collecting and sequencing the DNA or RNA cleavage products to identify the target cleavage sequence of a catalytic nucleic acid molecule, and e) contacting at least a portion of the sample with a bait nucleic acid molecule comprising the target cleavage sequence of the catalytic nucleic acid molecule to capture the catalytic nucleic acid molecule. In some embodiments, the method further comprises a step of sequencing the captured DNAzyme. In some embodiments, the single-strand phage is M13. In some embodiments, the method further comprises a step of contacting the ssDNA phage library with ssDNA endonuclease prior to contacting the phage-cloned ssDNA library with nucleic acid molecules under conditions that allow for the generation of DNA or RNA cleavage products by a catalytic nucleic acid molecule. In some embodiments, the test nucleic acid molecules are genonic DNA molecules. In some embodiments, the test nucleic acid molecules are mitochondrial DNA molecules. In some embodiments, the test nucleic acid molecules are RNA molecules.

In some embodiments, the method comprises the steps of: a) isolating endogenous single stranded DNA from at least one genomic or mitochondrial DNA sample; b) cloning a portion of the isolated ssDNA into a single-strand phage to generate a library of phage-cloned ssDNA: c) contacting the phage-cloned ssDNA library with test nucleic acid molecules under conditions that allow for the generation of DNA or RNA ligation products by a catalytic nucleic acid molecule; d) collecting and sequencing the DNA or RNA ligation products to identify the target cleavage sequence of a catalytic nucleic acid molecule, and e) contacting at least a portion of the sample with a bait nucleic acid molecule comprising the target cleavage sequence of the catalytic nucleic acid molecule to capture the catalytic nucleic acid molecule. In some embodiments, the method further comprises a step of sequencing the captured DNAzyme. In some embodiments, the single-strand phage is M13. In some embodiments, the method further comprises a step of contacting the ssDNA phage library with ssDNA endonuclease prior to contacting the phage-cloned ssDNA library with nucleic acid molecules under conditions that allow for the generation of DNA or RNA ligation products by a catalytic nucleic acid molecule. In some embodiments, the test nucleic acid molecules are genomic DNA molecules. In some embodiments, the test nucleic acid molecules are mitochondrial DNA molecules. In some embodiments, the test nucleic acid molecules are RNA molecules.

In some embodiments, the method comprises the steps of: a) isolating endogenous single stranded DNA from at least one genomic or mitochondrial DNA sample; b) cloning a portion of the isolated ssDNA into a single-strand phage to generate a library of phage-cloned ssDNA; c) contacting the phage-cloned ssDNA library with test nucleic acid molecules under conditions that allow for phosphorylation of the test nucleic acid molecules by a catalytic nucleic acid molecule; d) collecting and sequencing the phosphorylated nucleic acid molecules to identify the target phosphorylation site of a catalytic nucleic acid molecule, and e) contacting at least a portion of the sample with a bait nucleic acid molecule comprising the target phosphorylation site of the catalytic nucleic acid molecule to capture the catalytic nucleic acid molecule. In some embodiments, the method further comprises a step of sequencing the captured DNAzyme. In some embodiments, the single-strand phage is M13. In some embodiments, the method further comprises a step of contacting the ssDNA phage library with ssDNA endonuclease prior to contacting die phage-cloned ssDNA library with nucleic acid molecules under conditions that allow for the generation of phosphorylated nucleic acid molecules by a catalytic nucleic acid molecule. In some embodiments, the test nucleic acid molecules are genomic. DNA molecules. In some embodiments, the test nucleic acid molecules are mitochondrial DNA molecules. In some embodiments, the test nucleic acid molecules are RNA molecules.

### DNAzyme Compositions

In some embodiments, the invention provides compositions comprising a catalytic nucleic acid molecule and methods of use thereof for modifying at least one target nucleic acid. In some embodiments, the catalytic nucleic acid molecule comprises a DNAzyme.

In some embodiments, the DNAzyme is a genomic ssDNA sequence having enzymatic or catalytic activity (gDNAzyme). In some embodiments, the DNAzyme is a mitochondrial ssDNA sequence having enzymatic or catalytic activity (mtDNAzyme). In some embodiments the DNAzyme (e.g., gDNAzyme or mtDNAzyme) has catalytic activity against a target DNA molecule. In some embodiments the DNAzyme (e.g., gDNAzyme or mtDNAzyme) has catalytic activity against a target RNA molecule.

In some embodiments, the catalytic activity is a nuclease activity, ligase activity or phosphorylation activity. Therefore, in one embodiment, the invention relates to a composition comprising a DNAzyme having DNA cleavage activity, RNA cleavage activity, DNA ligation activity, RNA ligation activity, DNA phosphorylation activity or RNA phosphorylation activity.

In some embodiments, the DNAzyme is identified using a CHEX-seq assay, a DEAS assay, or a combination thereof. In some embodiments, the DNAzyme comprises an isolated nucleic acid molecule having catalytic activity. In some embodiments, the DNAzyme comprises a plasmid or vector comprising a nucleic acid molecule having catalytic activity.

### Modulator Compositions

In some embodiments, the invention provides compositions and methods for modulating the level or activity of a DNAzyme. In some embodiments, the DNAzyme is a genomic ssDNA sequence having enzymatic or catalytic activity (gDNAzyme). In some embodiments, the DNAzyme is a mitochondrial ssDNA sequence having enzymatic or catalytic activity (mtDNAzyme). In some embodiments the DNAzyme has catalytic activity against RNA.

In some embodiments the DNAzyme has catalytic activity against RNA. In some embodiments the DNAzyme has catalytic activity against DNA. In some embodiments, the catalytic activity is a nuclease activity, ligase activity or phosphorylation activity.

Modulators of the invention can activate or inhibit DNAzyme molecules. Inhibitors are compounds that, e.g., bind to, partially or totally block activity, decrease, prevent, delay activation, inactivate, desensitize, or down regulate the activity or expression of a DNAzyme. “Activators” are compounds that increase, open, activate, facilitate, enhance activation, sensitize, agonize, or up regulate activity of a DNAzyme, e.g., agonists. Inhibitors, activators, or modulators also include synthetic ligands, antagonists, agonists, antibodies, peptides, cyclic peptides, nucleic acids, antisense molecules, ribozymes, RNAi, microRNA, siRNA molecules, small organic molecules and the like. Assays for inhibitors and activators include, e.g., expressing DNAzyme in vitro, in cells, or cell extracts, applying putative modulator compounds, and then determining the functional effects on activity of the DNAzyme.

In some embodiments, the invention provides compositions for modulating the level or activity of one or more DNAzyme. In various embodiments, the present invention includes compositions for modulating the level or activity of one or more DNAzyme in a subject, a cell, a tissue, or an organ in need thereof.

In some embodiments, the compositions of the invention include compositions for treating or preventing diseases or disorders associated with an increased or decreased level or activity of one or more DNAzyme. In some embodiments, the compositions of the invention include compositions for treating or preventing diseases or disorders that would be benefitted by an increased or decreased level or activity of one or more DNAzyme. In one embodiment, diseases or disorders that can be treated using the compositions and methods of the invention include, but are not limited to, cancer, neurodegenerative disease, mitochondrial disease, diseases associated with DNA repair defects, diseases associated with telomere shortening, bacterial diseases and viral diseases.

In some embodiments, the compositions of the invention include compositions for modulating the level or activity of one or more DNAzyme in a plant cell. In one embodiment, the compositions or method of the invention may be beneficial for agricultural or environmental use. For example, the rate, and steady state levels of photosynthesis may be modulated using the compositions of the invention or plant dormancy may be modulated using the compositions of the invention.

In some embodiments, the compositions of the invention include compositions for modulating the level or activity of one or more DNAzyme in a biosensor. In some embodiments, the biosensor is for use in screening, diagnostics, and/or health monitoring. In one embodiment, the compositions or method of the invention may be beneficial for research, diagnostic, industrial or environmental use.

### Compositions for Modulating the Structure of DNAzymes

In some embodiments, disruption of a native three-dimensional (3D) structure within the genomic ssDNA region modulates the activity of the DNAzyme. Therefore, in some embodiments, the invention includes compositions and methods for disrupting a native 3D structure of a DNAzyme.

In some embodiments, the modulator comprises a clamping oligonucleotide which anneals to one or more internal regions of a genomic ssDNA region that functions as a DNAzyme. In one embodiment, the clamping oligonucleotide binds two separate regions within the genomic ssDNA region that functions as a DNAzyme, forcing the DNAzyme sequence to form a loop. In some embodiments, the formation of a loop modulates the activity level of the DNAzyme. Therefore, in some embodiments, the invention provides clamping oligonucleotides comprising a first binding region for binding to a first portion of a genomic ssDNA region that functions as a DNAzyme and a second binding region for binding to a first portion of a genomic ssDNA region that functions as a DNAzyme, wherein binding of the genomic ssDNA region by both the first and second binding regions of the clamping oligonucleotide generates a 3D structure within the genomic ssDNA region. In some embodiments, the 3D structure is a loop, a hairpin, a cruciform, a G4 quadruplex, or other unpaired DNA structures. In some embodiments, the formation of the 3D structure within the genomic ssDNA region modulates the activity of the DNAzyme.

In some embodiments, the clamping oligonucleotide may bind a target DNAzyme and cause a 3D structure to form, thereby preventing an activity of the DNAzyme. In some embodiments, the clamping oligonucleotide may bind a target DNAzyme, and cause a 3D structure to form thereby enhancing at least one activity of the DNAzyme.

In some embodiments, the clamping oligonucleotide binds to one or more internal regions of the DNAzyme forming a long loop which has decreased catalytic activity as compared to the native DNAzyme. In some embodiments, a long loop comprises more than 60, 65, 70, 75, 80 or more than 80 nucleotides.

In some embodiments, the clamping oligonucleotide binds to one or more internal regions of the DNAzyme forming a short loop which has increased catalytic activity as compared to the native DNAzyme. In some embodiments, a short loop comprises less than 60, 55, 50, 45, 40 or less than 40 nucleotides.

### Proteins and Peptides

In one embodiment, at least one composition for modulating the level or activity of one or more DNAzyme of the invention comprises a protein or peptide. The peptide of the present invention may be made using chemical methods. For example, peptides can be synthesized by solid phase techniques cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

In one embodiment, the peptide is made by recombinant means or by cleavage from a longer polypeptide. The composition of a peptide may be confirmed by amino acid analysis or sequencing.

The peptides can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction. A peptide or protein of the invention may be modified, e.g., phosphorylated, using conventional methods.

The peptides may include unnatural amino acids formed by post-translational modification or by introducing unnatural amino acids during translation. A variety of approaches are available for introducing unnatural amino acids during protein translation.

In one embodiment, at least one composition for modulating the level or activity of one or more DNAzyme of the invention comprises a cyclic peptide. Cyclization of peptide may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptide may be synthesized by conventional techniques. For example, the peptides or chimeric proteins may be synthesized by chemical synthesis using solid phase peptide synthesis or solution phase synthesis methods. By way of example, a peptide may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain a peptide fused to the selected protein or marker protein as described herein. Examples of proteins which may be used to prepare fusion proteins include immunoglobulins, glutathione-S-transferase (GST), hemagglutinin (HA), and truncated myc.

Peptides may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. Libraries may also be constructed by concurrent synthesis of overlapping peptides.

Peptides and proteins may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In one embodiment, at least one composition for modulating the level or activity of one or more DNAzyme comprises an antibody, or antibody fragment, that specifically binds to a target. In some embodiments, the antibody can modulate the level or activity of the DNAzyme.

### Small Molecule Therapeutic Agents

In various embodiments, at least one composition for modulating the level or activity of one or more DNAzyme is a small molecule chemical compound. When the modulator is a small molecule, the small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule therapeutic agent comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determine the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the agents depicted here, as well as the non-salt and non-solvate form of the agents, as is well understood by the skilled artisan. In some embodiments, the salts of the agents of the invention are pharmaceutically acceptable salts.

Where tautomeric forms may be present for any of the agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the agents described. The recitation of the structure or name herein is intended to embrace all possible stereoisomers of agents depicted. All forms of the agents are also embraced by the invention, such as crystalline or non-crystalline forms of the agents. Compositions comprising an agent of the invention are also intended, such as a composition of substantially pure agent, including a specific stereochemical form thereof, or a composition comprising mixtures of agents of the invention in any ratio, including two or more stereochemical forms, such as in a racemic or non-racemic mixture.

In some instances, small molecule therapeutic agents modulators are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by at least one chemical reactions. As such, an analog can be a structure having a structure similar to that of the small molecule therapeutic agents described herein or can be based on a scaffold of a small molecule therapeutic agent described herein, but differing from it in respect to certain components or structural makeup, which may have a similar or opposite action metabolically. An analog or derivative of any of a small molecule agent in accordance with the present invention can be used to treat a disease or disorder.

In one embodiment, the small molecule therapeutic agents described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having at least one hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Therapeutic Agents

In some embodiments, at least one composition for modulating the level or activity of one or more DNAzyme an isolated nucleic acid. In various embodiments, the isolated nucleic acid molecule is a DNA molecule or an RNA molecule. In various embodiments, the isolated nucleic acid molecule is a cDNA, mRNA, miRNA, siRNA, antagomir, antisense molecule, or CRISPR guide RNA molecule. In one embodiment, the isolated nucleic acid molecule encodes a therapeutic peptide. In some embodiments, the therapeutic agent is an siRNA, miRNA, sgRNA or antisense molecule, which inhibits a targeted nucleic acid. In one embodiment, the nucleic acid comprises a promoter/regulatory sequence such that the nucleic acid is capable of directing expression of the nucleic acid. Thus, in one embodiment at least one composition for modulating the level or activity of one or more DNAzyme, of the invention comprises an expression vector, and the invention comprises a method for the introduction of exogenous DNA into cells with concomitant expression of the exogenous DNA in the cells.

In one embodiment of the invention, DNAzyme, can be inhibited by way of inactivating and/or sequestering the DNAzyme. As such, inhibiting the activity of the targeted gene or protein can be accomplished by using an antisense nucleic acid molecule or a nucleic acid molecule encoding a transdominant negative mutant.

In one embodiment, siRNA is used to decrease the level of a DNAzyme. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. In one embodiment, an siRNA comprises a chemical modification that aids in intravenous systemic delivery. Optimizing siRNAs involves consideration of overall G/C content, C/T content at the termini, Tm and the nucleotide content of the 3′ overhang. Therefore, the present invention also includes methods of decreasing levels of expression products (i.e., mRNA and protein) of a target gene using RNAi technology.

In one embodiment, at least one composition for modulating the level or activity of one or more DNAzyme of the invention comprises a vector comprising an siRNA or antisense polynucleotide. In one embodiment, the siRNA or antisense polynucleotide is capable of inhibiting the expression of a target polypeptide. The incorporation of a desired polynucleotide into a vector and the choice of vectors is well-known in the art.

In certain embodiments, the expression vectors described herein encode a short hairpin RNA (shRNA) therapeutic agent. shRNA molecules are well known in the art and are directed against the mRNA of a target, thereby decreasing levels of the expression products (i.e., mRNA and protein) of the target gene of interest. In certain embodiments, the encoded shRNA is expressed by a cell, and is then processed into siRNA. For example, in certain instances, the cell possesses native enzymes (e.g., dicer) that cleaves the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells contacted with at least one composition for modulating the level or activity of one or more DNAzyme. In one embodiment, the selectable marker may be carried on a separate piece of DNA. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one embodiment, at least one composition for modulating the level or activity of one or more DNAzyme may be in the form of a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art. In a particular embodiment, the vector is a vector useful for transforming animal cells.

In one embodiment, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, and other techniques known in the art. Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, 3-galactosidase, chloramphenicol acetyltransferase, firefly luciferase, or an immunoglobulin or portion thereof such as the Fc portion of an immunoglobulin, such as IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like.

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art. Antisense nucleic acids are DNA or RNA molecules that are complementary to at least a portion of a specific mRNA molecule. In the cell, antisense nucleic acids hybridize to the corresponding mRNA, forming a double-stranded molecule thereby inhibiting the translation of genes.

The use of antisense methods to inhibit the translation of genes is known in the art. Such antisense molecules may be provided to the cell via genetic expression using DNA encoding the antisense molecule.

Alternatively, antisense molecules of the invention may be made synthetically and then provided to the cell. In one embodiment, antisense oligomers of between about 10 to about 30 nucleotides are used since they are easily synthesized and introduced into a target cell. Synthetic antisense molecules contemplated by the invention include oligonucleotide derivatives known in the art which have improved biological activity compared to unmodified oligonucleotides.

In one embodiment, the modulator may comprise at least one component of a CRISPR-Cas system, where a guide RNA (gRNA) targeted to a DNAzyme, and a CRISPR-associated (Cas) peptide form a complex to induce mutations within the targeted gene. In one embodiment, the therapeutic agent comprises a gRNA or a nucleic acid molecule encoding a gRNA. In one embodiment, the therapeutic agent comprises a Cas peptide or a nucleic acid molecule encoding a Cas peptide.

DNAzymes with Porphyrin Metalation Activity

In some embodiments, the invention provides a DNAzyme having porphyrin metalation activity. In some embodiments, the DNAzyme comprises a sequence as set forth in Table 2 designated as TATDN2P1short, TATDN2P1long or RPL7AP61. In some embodiments, the DNAzyme comprises a complex comprising a DNAzyme comprises a sequence as set forth in Table 2 designated as TATDN2P1short or TATDN2P1long hybridized to a clamping oligonucleotide comprising a sequence as set forth in Table 2 designated as opTATDN2Planneal-short-loop. In some embodiments, the DNAzyme comprises a complex comprising a DNAzyme comprises a sequence as set forth in Table 2 designated as TATDN2P1short or TATDN2P1long hybrizided to a clamping oligonucleotide comprising a sequence as set forth in Table 2 designated as opTATDN2Planneal-long-loop.

### Methods of Modulating Porphyrin Metalation

In some embodiments, the invention includes methods for modulating the level or activity of porphyrin metalation in a sample. In some embodiments, the sample comprises a cell. In some embodiments the cell is a eukaryotic cell. In some embodiments, the cell is a mammalian cell. In some embodiments, the cell is a human cell. In some embodiments, the method comprises administering a DNAzyme having porphyrin metalation activity to the cell or organism. In some embodiments, the DNAzyme having porphyrin metalation activity is a DNAzyme comprising a sequence set forth in Table 2 designated as TATDN2P1short, TATDN2P1long or RPL7AP61. In some embodiments, the method comprises administering a clamping oligonucleotide which generates, stabilizes, or destabilizes a loop structure in a DNAzyme having porphyrin metalation activity. In some embodiments, the clamping oligonucleotide comprises a sequence as set forth in Table 2 designated as opTATDN2Planneal-short-loop or opTATDN2Planneal-long-loop. In some embodiments, the method comprises administering a complex comprising a DNAzyme comprises a sequence as set forth in Table 2 designated as TATDN2P1short or TATDN2P1long hybridized to a clamping oligonucleotide comprising a sequence as set forth in Table 2 designated as opTATDN2Planneal-short-loop. In some embodiments, the method comprises administering a complex comprising a DNAzyme comprises a sequence as set forth in Table 2 designated as TATDN2P1short or TATDN2P1long hybridized to a clamping oligonucleotide comprising a sequence as set forth in Table 2 designated as opTATDN2Planneal-long-loop.

### Pharmaceutical Compositions

The present invention provides pharmaceutical compositions comprising at least one agent for use in the methods of the invention. The relative amounts of the agent(s), any pharmaceutically acceptable carrier, and any additional ingredients in a pharmaceutical composition of the invention will vary, depending upon the identity, size, and condition of the subject treated and further depending upon the route by which the composition is to be administered.

The formulations of the pharmaceutical compositions described herein may be prepared by any method known or hereafter developed in the art of pharmacology. In general, such preparatory methods include the step of bringing the active ingredient into association with a carrier or at least one other accessory ingredients. Said compositions may comprise additional medicinal agents, pharmaceutical agents, carriers, buffers, adjuvants, dispersing agents, diluents, and the like depending on the intended use and application.

Examples of suitable pharmaceutical carriers, excipients and/or diluents are well known in the art and include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Pharmaceutically acceptable carriers for liquid formulations are aqueous or non-aqueous solutions, suspensions, emulsions or oils, Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, turmeric oil, fish-liver oil, another marine oil, or a lipid from milk or eggs.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Compositions comprising such carriers can be formulated by well-known conventional methods. Suitable carriers may comprise any material which, when combined with the biologically active compound of the invention, retains the biological activity. Preparations for parenteral administration may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles may include sodium chloride solution, Ringer's dextrose, dextrose and sodium chloride, lactated Ringer's, or fixed oils. Intravenous vehicles may include fluid and nutrient replenishes, electrolyte replenishers (such as those based on Ringer's dextrose), and the like. Preservatives and other additives may also be present including, for example, antimicrobials, anti-oxidants, chelating agents, and inert gases and the like, in addition, the pharmaceutical composition of the present invention might comprise proteinaceous carriers, e.g., serum albumin or immunoglobulin, in some embodiments of human origin.

At least one composition for modulating the level or activity of one or more DNAzyme may be administered alone, or in combination with other drugs and/or agents as pharmaceutical compositions. The composition may contain at least one added materials such as carriers and/or excipients. As used herein, “carriers” and “excipients” generally refer to substantially inert, non-toxic materials that do not deleteriously interact with other components of the composition. These materials may be used to increase the amount of solids in particulate pharmaceutical compositions, such as to form a powder of drug particles. Examples of suitable carriers include water, silicone, gelatin, waxes, and the like.

Examples of normally employed “excipients,” include pharmaceutical grades of mannitol, sorbitol, inositol, dextrose, sucrose, lactose, trehalose, dextran, starch, cellulose, sodium or calcium phosphates, calcium sulfate, citric acid, tartaric acid, glycine, high molecular weight polyethylene glycols (PEG), and the like and combinations thereof. In one embodiment, the excipient may also include a charged lipid and/or detergent in the pharmaceutical compositions. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, for example, TWEEN surfactants (Atlas Chemical Industries, Wilmington, Del.), polyoxyethylene ethers, for example, Brij®, pharmaceutically acceptable fatty acid esters, for example, lauryl sulfate and salts thereof (SDS), and the like. Such materials may be used as stabilizers and/or anti-oxidants. Additionally, they may be used to reduce local irritation at the site of administration.

In at least one embodiment, the composition is formulated in a lyophilized form. In certain embodiments, the lyophilized formulation of the composition allows for maintaining structure and achieving remarkably superior long-term stability conditions which might occur during storage or transportation of the composition.

### Methods of Modulating DNAzyme Activity

In some embodiments, the invention provides methods of modulating the level or activity of one or more DNAzyme. In some embodiments, the DNAzyme is a gDNAzyme. In some embodiments, the DNAzyme is a mtDNAzyme.

In various embodiments, the present invention includes methods of modulating the level or activity of one or more DNAzyme in a subject, a cell, a tissue, or an organ in need thereof.

In some embodiments, the invention provides a method for treating or preventing diseases or disorders associated with an increased or decreased level or activity of one or more DNAzyme. In some embodiments, the invention provides a method for treating or preventing diseases or disorders that would be benefitted by an increased or decreased level or activity of one or more DNAzyme. In one embodiment, diseases or disorders that can be treated using the compositions and methods of the invention include, but are not limited to, cancer, neurodegenerative disease, mitochondrial disease, diseases associated with DNA repair defects, diseases associated with telomere shortening, bacterial diseases and viral diseases.

In some embodiments, the invention includes methods for modulating the level or activity of one or more DNAzyme in a plant cell. In one embodiment, the methods of the invention are beneficial for agricultural or environmental use.

In some embodiments, the invention includes methods for modulating the level or activity of one or more DNAzyme in a biosensor. In some embodiments, the biosensor is for use in screening, diagnostics, and/or health monitoring. In one embodiment, the method of the invention is beneficial for research, diagnostic, industrial or environmental use.

It will be appreciated by one of skill in the art, when armed with the present disclosure including the methods detailed herein, that the invention is not limited to treatment of diseases or disorders that are already established. Particularly, the disease or disorder need not have manifested to the point of detriment to the subject; indeed, the disease or disorder need not be detected in a subject before treatment is administered. That is, significant signs or symptoms of diseases or disorders do not have to occur before the present invention may provide benefit. Therefore, the present invention includes a method for preventing diseases or disorders, in that a composition, as discussed previously elsewhere herein, can be administered to a subject prior to the onset of diseases or disorders, thereby preventing diseases or disorders.

One of skill in the art, when armed with the disclosure herein, would appreciate that the prevention of a disease or disorder, encompasses administering to a subject a composition as a preventative measure against the development of, or progression of, a disease or disorder.

One of skill in the art will appreciate that the compositions of the invention can be administered singly or in any combination. Further, the compositions of the invention can be administered singly or in any combination in a temporal sense, in that they may be administered concurrently, or before, and/or after each other. One of ordinary skill in the art will appreciate, based on the disclosure provided herein, that the compositions of the invention can be used to prevent or to treat a disease or disorder, and that a composition can be used alone or in any combination with another composition to affect a therapeutic result. In various embodiments, any of the compositions of the invention described herein can be administered alone or in combination with other modulators of other molecules associated with diseases or disorders.

In one embodiment, the invention includes a method comprising administering a combination of compositions described herein. In certain embodiments, the method has an additive effect, wherein the overall effect of the administering a combination of compositions is approximately equal to the sum of the effects of administering each individual inhibitor. In other embodiments, the method has a synergistic effect, wherein the overall effect of administering a combination of compositions is greater than the sum of the effects of administering each individual composition.

The method comprises administering a combination of composition in any suitable ratio. For example, in one embodiment, the method comprises administering two individual compositions at a 1:1 ratio. However, the method is not limited to any particular ratio. Rather any ratio that is shown to be effective is encompassed.

### Methods of Treatment

In one embodiment, the method of the invention includes identifying a subject as having a disease or disorder that would benefit from treatment with at least one DNAzyme, and administering an isolated DNAzyme to the subject. In some embodiments, the DNAzyme is a gDNAzyme. In some embodiments, the DNAzyme is a mtDNAzyme. In some embodiments, the DNAzyme has DNA cleavage activity, RNA cleavage activity, DNA ligation activity, RNA ligation activity, DNA phosphorylation activity or RNA phosphorylation activity.

In one embodiment, the method of the invention includes a) identifying a subject as having a disease or disorder that would benefit from treatment with at least one agent for modulating the activity of at least one DNAzyme and b) administering an agent for modulating the level of activity of at least one DNAzyme to the subject. In some embodiments, the DNAzyme is a gDNAzyme. In some embodiments, the DNAzyme is a mtDNAzyme.

In one embodiment, the invention relates to methods of administering a composition comprising an agent for decreasing the level or activity of at least one DNAzyme to the subject. In one embodiment, the invention relates to methods of administering a composition comprising an agent for increasing the level or activity of at least one DNAzyme to the subject.

In various embodiments, at least one agent for modulating the activity of at least one DNAzyme, is administered to a subject in need in a wide variety of ways. In various embodiments, at least one agent for modulating the activity of at least one DNAzyme, is administered orally, intraoperatively, intratumorally, intravenously, intravascularly, intramuscularly, subcutaneously, intracerebrally, intraperitoneally, by soft tissue injection, by surgical placement, by arthroscopic placement, and by percutaneous insertion, e.g., direct injection, cannulation or catheterization. Any administration may be a single administration of a composition of invention or multiple administrations. Administrations may be to single site or to more than one site in the subject being treated. Multiple administrations may occur essentially at the same time or separated in time.

In certain embodiments, the composition of the invention is administered during surgical resection or debulking of a tumor or diseased tissue. For example, in subjects undergoing surgical treatment of diseased tissue or tumor, the composition may be administered to the site in order to further treat the tumor.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous (i.v.) injection, or intraperitoneally.

The composition comprising at least one agent as described herein can be incorporated into any formulation known in the art. For example, at least one agent for modulating the activity of at least one DNAzyme may be incorporated into formulations suitable for oral, parenteral, intravenous, subcutaneous, percutaneous, topical, buccal, or another route of administration. Suitable compositions include, but are not limited to, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

In the method of treatment, the administration of the composition of the invention may be for either “prophylactic” or “therapeutic” purpose. When provided prophylactically, the composition of the present invention is provided in advance of any sign or symptom, although in particular embodiments the invention is provided following the onset of at least one sign or symptom to prevent further signs or symptoms from developing or to prevent present signs or symptoms from becoming more severe. The prophylactic administration of the composition serves to prevent or ameliorate subsequent signs or symptoms. When provided therapeutically, the pharmaceutical composition is provided at or after the onset of at least one sign or symptom. Thus, the present invention may be provided either prior to the anticipated exposure to a disease-causing agent or disease state or after the initiation of the disease or disorder.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: CHEX-Seq Detects Single-Cell Genomic ssDNA with Catalytical Capacity

Genomic DNA (gDNA) undergoes structural interconversion between single-stranded and double-stranded states during transcription, DNA repair and replication, which is critical for cellular homeostasis and response to external stimuli. However, surveying the single-stranded DNA (ssDNA) chromatin landscape at a genome-wide and single-cell level has not previously been possible.

The CHEX-seq (CHromatin EXposed) assay identifies single-stranded open chromatin in situ in individual, formalin-fixed cells. CHEX-seq uses 3′-terminal blocked, light-activatable probes to prime the copying of ssDNA into complementary DNA (cDNA) that is sequenced, allowing for profiling of the single-stranded chromatin status. CHEX-seq was benchmarked against other open-chromatin assays in human K562 cells, and utilities were demonstrated in dispersed primary cultures of mouse and human brain cells as well as immunostained spatially localized neurons in brain sections. The amount of ssDNA is rapidly and dynamically regulated in response to acute mitogen perturbation.

Further, CHEX-seq also identifies single-stranded regions of mitochondrial DNA in single cells. Surprisingly, with the ability to query ssDNA in single cells, CHEX-seq identified single-stranded loci in mouse and human gDNA that catalyze porphyrin metalation in vitro suggesting an unprecedented catalytic activity for genomic ssDNA. Together, these experiments describe the application of CHEX-seq to exploring ssDNA open chromatin in fixed dispersed neurons and astrocytes, and in site single neurons preserving the cellular microenvironment.

The materials and methods used in these experiments are now described.

### Human Brain Tissue

Human brain tissue was collected using standard operating procedures for enrollment and consent of patients. Briefly, an en bloc sample of brain (typically 5×5×5 mm) was obtained from cortex that was resected as part of neurosurgical procedures for the treatment of epilepsy or brain tumors. This tissue was immediately transferred to a container with ice-cold oxygenated artificial CSF (KCl 3 mM, NaH2PO4 2.5 mM, NaHCO₃26 mM, glucose 10 mM, MgCl2-6H₂O 1 mM, CaCl₂)-2H₂O 2 mM, sucrose 202 mM, with 5% CO2 and 95% O₂ gas mixture) for transfer to the laboratory. Tissues arrived in the laboratory ˜10 minutes post excision. The brain tissues were then processed for cell culturing and fixation.

### Cell Culturing/Preparation and Fixation

K562 cells were obtained from ATCC and cultured in RPMI 1640 medium (Invitrogen) with 10% FBS and penicillin-streptomycin in a T75 flask at 37° C. in 5% CO2 for 2-3 days. The cultured cells were transferred to a 50 ml tube and 16% paraformaldehyde (final 1%) was added for 10 mins at room temperature to fix the cells. After fixation, 1 M glycine (final 200 mM) with RPMI 1640 medium was used to quench for 10 mins followed by centrifugation at 300×g for 5 mins. The supernatant was discarded, and 3 mL of PBS were added to the pellet and then mixed by gently pipetting up and down 10-15 times using a fire-polished glass-pipette, to prevent cell clumping, and centrifuged at 300×g for 5 mins. The 100 μl cell pellet was attached to 18 mm gridded coverslips by incubating them for 2 hr at room temperature. The samples were treated with PBS (w/o Ca++, Mg++) containing 0.01% Triton X-100 for 10 mins and then washed with PBS (w/o Ca++, Mg++) 3 times for 3 mins.

To prepare human neuronal cell cultures, adult human brain tissue was placed in the papain (20 U, Worthington Biochemical) solution to dissociate at 37° C. for 30 to 40 mins and followed by ovomucoid (a papain inhibitor, 10 mg/ml, Worthington Biochemical) to stop the enzymatic dissociation (Spaethling et al., 2017, Cell Rep., 18, 791-803; Buchhalter et al., 1991, Brain Res Bull. 26, 333-8). The tissue was triturated with a fire-polished glass Pasteur pipette. The cloudy cell suspension was carefully transferred to a new tube and centrifuged at 300×g for 5 mins at room temperature. The cells were counted in an Autocounter (Invitrogen). Cells were plated on poly-L-lysine-coated (0.1 mg/ml, Sigma-Aldrich) 12 mm coverslips at a density of 3×10⁴ cells/coverslip. Cultures were incubated at 37° C., 95% humidity, and 5% CO2 in neuronal basal medium (Neurobasal A, Gibco), serum-free supplement (B-27, Gibco) and 1% penicillin/streptomycin (Thermo-Fisher Scientific). Dispersed mouse neuron/astrocyte cultures were prepared following published protocols (Buchhalter et al., 1991, Brain Res Bull. 26, 333-8). Dispersed cells were fixed using 4% paraformaldehyde for 10 min at room temperature. Three washes were then performed with 1×PBS. The cells were permeabilized with 0.1% Triton-X100 for 10 min at room temperature followed by another three washes with 1×PBS. For select experiments, K562 cells were treated with 16 mM TPA (12-O-tetra-decanoylphorbol-13-acetate) for 15 mins, 1, 2, and 24 hrs.

### Mouse Brain Tissue Sections

A three-month-old male mouse was anaesthetized with halothane, euthanized by thoracotomy, then subjected to cardiac perfusion with 5 ml PBS followed by 20 ml PBS/4% paraformaldehyde. The brain was removed and post fixed at 4° C. for 16 hr., then rinsed in PBS and sectioned in the coronal plane at 100 m on a vibratome (Leica VT-1000s). Sections including the hippocampus were then subjected to immunofluorescence labeling with chicken anti-MAP2 antibody (1:1000; Ab 5392; Abcam) followed by Alexa 488 conjugated goat anti-chicken secondary antibody (1:400; ab150169; Abcam).

### CHEX-Seq Probe Synthesis

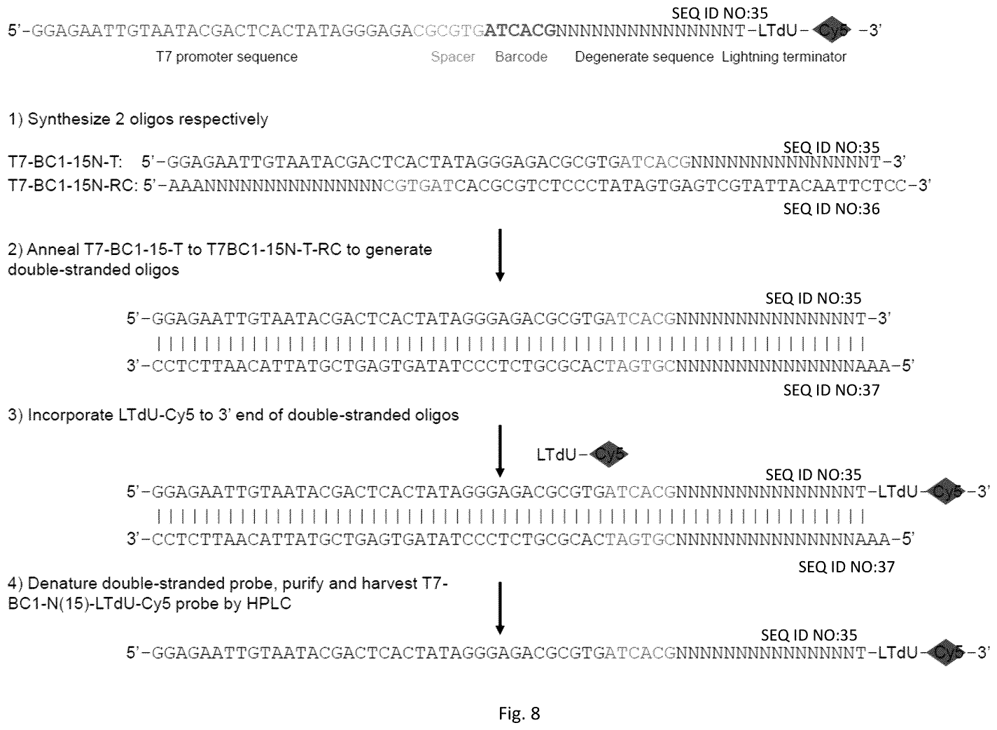

HPLC-purified probe oligo and its complimentary oligo were purchased. A template-dependent DNA polymerase incorporation assay was employed to extend Cy5-dye-labeled Lightning Terminator™ (Agilent®) to the 3′ end of probe oligo: (1) 5 μM of probe oligo, 25 μM complimentary oligo, 50 μM of Cy5-labeled Lightning Terminator™, 4 mM MgSO4, and 0.1 U/μL of Therminator™ (New England Biolabs®) were mixed in 1× ThermoPol® buffer, (2) the mix was heated to 80° C. for 45 sec and (3) then incubated for 5 mins at each of 60° C., 55° C., 50° C., 45° C., 40° C., 35° C., 30° C. and 25° C. The incorporation product was purified on the 1260 Infinity reverse phase HPLC (Agilent Technologies®) using the XTerra® MS C18 Prep column (Waters®). The purified product solution was concentrated to approximately 250 μL using the Vacufuge® (Eppendorf®) followed by denaturation into single-stranded oligo with equal volume of 0.2 M NaOH. HPLC purification and concentration were repeated using the same conditions for collection of the Lighting Terminator™-labeled single-stranded probe. The final product was dissolved into 1×PBS, and the concentration was determined by measuring Cy5 absorbance at 647 nm (FIG. 8).

CHEX-Seq Probe Annealing. Imaging and Photoactivation

After fixation and permeabilization, the cells and brain sections were incubated with CHEX-seq probe (170 nM) in TES buffer (10 mM Tris, 1 mM EDTA, 150 mM NaCl) for 1 hr at room temperature. The samples were then washed with 1×PBS (w/o Ca⁺⁺, Mg⁺⁺) 3 times for 3 min. After CHEX-seq probe annealing and washing, the samples were transferred to the imaging chamber with 1×PBS (w/o Ca⁺⁺, Mg⁺⁺). All images and photoactivations were performed using a Carl Zeiss 710 Meta confocal microscope (20× water-immersion objectives, NA 1.0). CHEX-seq probe annealing was confirmed by exciting at 633 nm and emission was detected at 640-747 nm. The photoactivation was performed using the 405 nm (UV) laser at 60% power and 6.30 s per pixel.

### First Strand DNA Synthesis In Situ and Single Cell Harvesting

After photoactivation in each individual cell's nucleus, a master mix containing DNA polymerase I and 1^{st} strand DNA synthesis buffer was added to the cells and incubated for 1 hr at room temperature. Subsequently, the single cells containing synthesized cDNA were harvested using a glass micropipette under using a Zeiss® 710 confocal microscope (Carl Zeiss®) for visualization.

### Linear Amplification of Nucleosome Free Areas of Chromatin

### 1^{st} Strand DNA Synthesis and Poly G Tailing at 3′ End

After harvesting single cells, the in situ synthesized cDNA was removed by adding fresh prepared 0.1 N NaOH and incubating the sample for 5 min at RT followed by neutralization with 1 M Tris (pH 7.5). After ethanol precipitation, the 1st strand DNA was resuspended in nuclease free water. Subsequently, poly(G) was added to the 3′ end using terminal deoxynucleotidyl transferase (TdT) (Invitrogen).

2^{nd} strand DNA synthesis and round 1 linear RNA amplification

2^{nd} strand DNA was synthesized using DNA polymerase I for 2 hours at 16° C. after priming with custom App-RC-polyC primer (FIG. 8). RNA was amplified using linear in vitro transcription from T7 RNA polymerase promoter incorporated into the double-stranded DNA with Ambion MEGAscript T7 In Vitro Transcription (IVT) Kit.

Round 2 1^{st} and 2^{nd} Strand DNA Synthesis and PCR Amplification

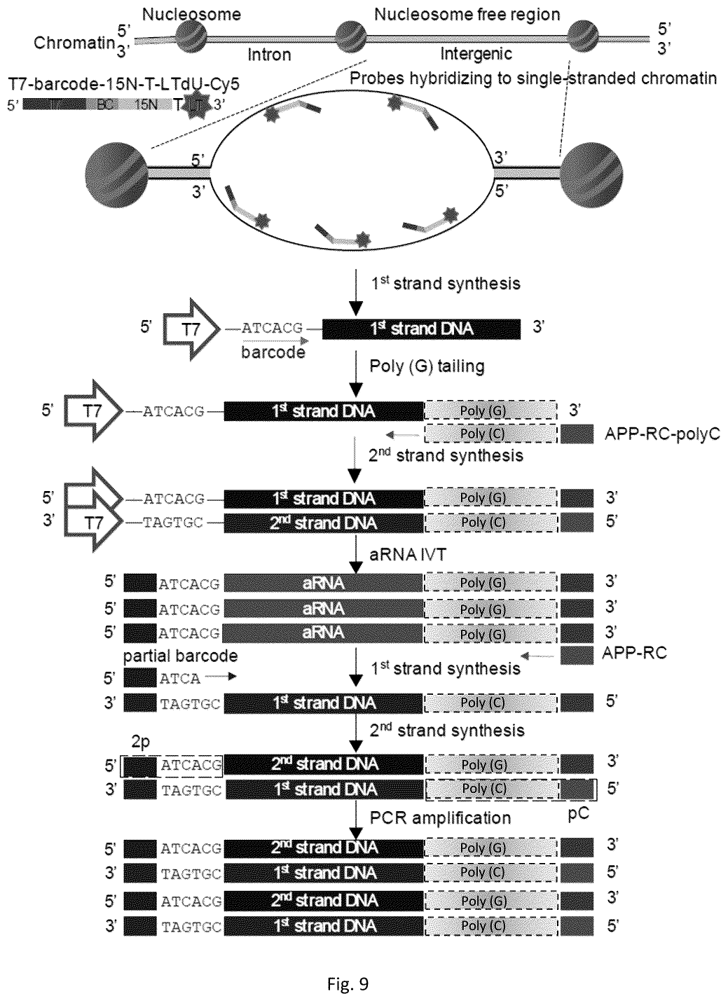

After cleanup IVT reaction, 1^{st} strand DNA was reverse transcribed from aRNA using Superscript III using a custom App-RC primer and 2nd strand DNA was synthesized using DNA Polymerase 1 with a custom XXbpPBCYY primer (XX=16-18, YY=1, 15-24; for complete list see Table 1). Subsequently, the double-stranded blunt ended DNA was amplified using custom primers XXbpPBCYY/App-RC (Table 1) following PCR condition: 98° C. for 30 sec; thermocycling at 98° C. for 10 sec, 50° C. for 30 sec, 72° C. for 30 sec for 27 cycles; extension at 72° C. for 2 mins and was then used for library construction. To distinguish CHEX-seq priming from endogenous PCR priming, the PCR primers were designed with their 3′ ends clipped up to 4 bp. Samples for the control experiments were processed with the same procedure except no CHEX-seq probe was applied, and 2^{nd} round 2^{nd} strand DNA PCR amplification was performed with custom primers 18bpPBC14/App-RC (FIG. 9).

Table 1: List of CHEX-seq probes, barcodes, and primers. The bold font denotes the barcode (full or 3′ end-clipped partial version); the underlined font denotes the T7 promoter in CHEX-seq probes; the first spacer is between the T7 promoter and the barcode, and the second spacer in dual-spacer primers (i.e., 510-518) is in lower case.

| | SEQ | | |
|---|---|---|---|
| | ID | | |
| ID | NO: | Name | Sequence |
| 302 | 1 | CHEX-App-RC-polyC | 5′-GCGCCATTGACCAGGATTITCCCCCCCCCCCCCC-3′ |
| 303 | 2 | CHEX-App-RC | 5′-GCGCCATTGACCAGGATTTTC-3′ |
| 505 | 3 | CHEX-20bpBC1 | 5′-TAGGGAGACGCGTGATCACG-3′ |
| 510 | 4 | CHEX-15NT-2S-BC15 | 5′-GGAGAATTGTAATACGACTCACTATAGGGAGACGCGTGATGTCAacaatgaggaNNNNNNN |
| | | | NNNNNNNNT-3′ |
| 511 | 5 | CHEX-15NT-2S-BC16 | 5′-GGAGAATTGTAATACGACTCACTATAGGGAGACGCGTGCCGTCCacaatgaggaNNNNNNN |
| | | | NNNNNNNNT-3′ |
| 512 | 6 | CHEX-15NT-2S-BC17 | 5′-GGAGAATTGTAATACGACTCACTATAGGGAGACGCGTGGTAGAGacaatgaggaNNNNNNN |
| | | | NNNNNNNNT-3′ |
| 513 | 7 | CHEX-15NT-2S-BC19 | 5′-GGAGAATTGTAATACGACTCACTATAGGGAGACGCGTGGTGAAAacaatgaggaNNNNNNN |
| | | | NNNNNNNNT-3′ |
| 514 | 8 | CHEX-15NT-2S-BC20 | 5′-GGAGAATTGTAATACGACTCACTATAGGGAGACGCGTGGTGGCCacaatgaggaNNNNNNN |
| | | | NNNNNNNNT-3′ |
| 515 | 9 | CHEX-15NT-2S-BC21 | 5′-GGAGAATTGTAATACGACTCACTATAGGGAGACGCGTGGTTTCGacaatgaggaNNNNNNN |
| | | | NNNNNNNNT-3′ |
| 516 | 10 | CHEX-15NT-2S-BC22 | 5′-GGAGAATTGTAATACGACTCACTATAGGGAGACGCGTGCGTACGacaatgaggaNNNNNNN |
| | | | NNNNNNNNT-3′ |
| 517 | 11 | CHEX-15NT-2S-BC23 | 5′-GGAGAATTGTAATACGACTCACTATAGGGAGACGCGTGGAGTGGacaatgaggaNNNNNNN |
| | | | NNNNNNNNT-3′ |
| 518 | 12 | CHEX-15NT-2S-BC24 | 5′-GGAGAATTGTAATACGACTCACTATAGGGAGACGCGTGGGTAGCacaatgaggaNNNNNNN |
| | | | NNNNNNNNT-3′ |
| 505b | 13 | CHEX-18bpPBC1 | 5′-TAGGGAGACGCGTGATCA-3′ |
| | | (short BC1) | |
| 505c | 14 | CHEX-17bpPBC1 | 5′-TAGGGAGACGCGTGATC-3′ |
| | | (short BC1) | |
| 505d | 15 | CHEX-16bpPBC1 | 5′-TAGGGAGACGCGTGAT-3′ |
| | | (short BC1) | |
| 507b | 16 | CHEX-18bpPBC14 | 5′-TAGGGAGACGCGTGAGTT-3′ |
| 510b | 17 | CHEX-18bpPBC15 | 5′-TAGGGAGACGCGTGATGT-3′ |
| 511b | 18 | CHEX-18bpPBC16 | 5′-TAGGGAGACGCGTGCCGT-3′ |
| 512b | 19 | CHEX-18bpPBC17 | 5′-TAGGGAGACGCGTGGTAG-3′ |
| 513b | 20 | CHEX-18bpPBC19 | 5′-TAGGGAGACGCGTGGTGA-3′ |
| 514b | 21 | CHEX-18bpPBC20 | 5′-TAGGGAGACGCGTGGTGG-3′ |
| 515b | 22 | CHEX-18bpPBC21 | 5′-TAGGGAGACGCGTGGTTT-3′ |
| 516b | 23 | CHEX-18bpPBC22 | 5′-TAGGGAGACGCGTGCGTA-3′ |
| 517b | 24 | CHEX-18bpPBC23 | 5′-TAGGGAGACGCGTGGAGT-3′ |
| 518b | 25 | CHEX-18bpPBC24 | 5′-TAGGGAGACGCGTGGGTA-3′ |
| 529n | 26 | CHEX-18bp-NBC | 5′-AGACGCagaagagcaGTG-3′ |

### Sequencing Library Preparation

Illumina® TruSeq® Nano DNA Library Preparation Kit was used with modifications. The entire second round PCR amplified double-stranded DNA was used as input. After converting DNA fragment into blunt ends with End Repair Mix, base “A” was added; sequence adapters were ligated. DNA inserts were amplified with PCR. High-throughput sequencing was done on Illumina® NextSeq® 500.

### Data Preprocessing

Raw FASTQ mates were concatenated by the read pair, then multiple copies of the same concatenated sequence were reduced to a unique one. Any identical read pairs from two different samples were defined as the shared reads and removed. Substrings from the primer 2p and pC were searched and trimmed (if found) sequentially (first 2p, then pC) (FIG. 9, Table 1). After trimming, reads shorter than 10 bp were discarded. Subsequently, poly Ns were trimmed (if found) from the ends. The resultant reads shorter than 10 bp were discarded. Read pairs were then aligned by STAR (Dobin et al., in press, doi:10.1002/0471250953.bi1114s51), to the GENCODE GRCh38 genome (Harrow et al., 2012, Genome Res., 22, 1760-1774; Frankish et al., 2019, Nucleic Acids Res., 47, D766-D773) (for human samples) or the UCSC mm10 genome (Kent et al., 2002, Genome Res., 12, 996-1006; Lee et al., 2022, Nucleic Acids Res., 50, D1115-D1122 (for mouse samples), with splicing junction disabled. The joint set of mapping criteria are (1) minimal mapping length 10; (2) minimal score normalized to read length 0.4; (3) minimal matched bases normalized to read length 0.4; (4) maximal mismatched bases normalized to mapped length (i.e., per-base mismatch rate) 0.1. The alignments were further filtered for the primary location and non-PCR duplicates called by Picard (github.com/broadinstitute/picard). Reads with a non-overlapping mapped length below 20 bp or mismatch rate greater than 0.1 were excluded.

To eliminate possible contamination, each human or mouse sample was aligned to alternative genomes: for human samples, mouse, bacteria (top 20 species) and mycoplasma; for mouse samples, human, bacteria (top 20 species) and mycoplasma. For each sample of a given species, the reads that aligned better to an alternative genome than the target genome were defined as contaminants and removed. To remove misalignments, 20 bp single-end reads were synthesized and mapped them to human or mouse reference using STAR and the same parameter setting. Genomic regions where reads could not be faithfully mapped (i.e., to a wrong chromosome or to the same chromosome but >10 bp off) were then filtered out from the CHEX-seq reads. In addition, human and mouse blacklist regions were obtained from ENCODE (Amemiya et al., 2019, Genome. Sci. Rep., 9, 1-5) and CHEX-seq reads whose barcode overlap with the blacklist regions were excluded.

CHEX-Seg ssDNA Calls and Priming Counts

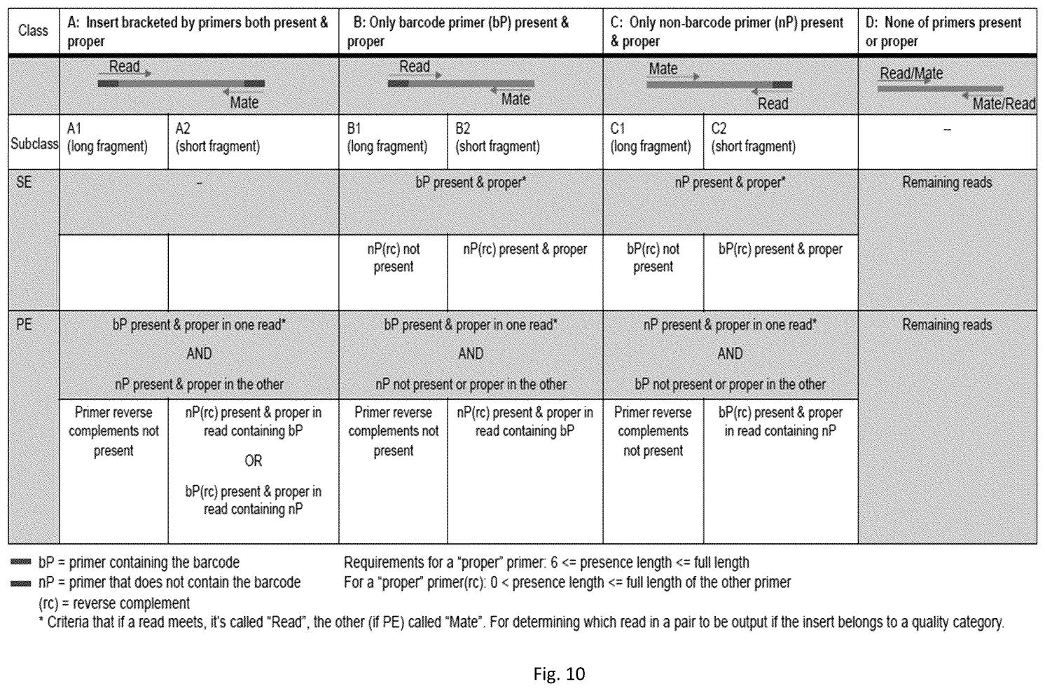

Based on the presence and format of 5′ end T7 barcode and 3′ end pC primers, the alignments were classified into four categories: A, B, C, and D, among which A to C were divided into subclass 1 or 2, depending on the presence of read-through or not (FIG. 10). For the sake of a balanced sensitivity and specificity, class A (both proper 2p and pC primer) and B reads (proper 2p/barcode primer only) were defined as good quality and merged as the AB reads. Then barcode locations were tracked in 20 bp bins across the genome. For each sample with k cells, the number of reads whose barcodes hit the same bin were reduced to a maximum 2×k, given the diploidy of the samples in this study. The barcode/primer quality (A1>A2>B1>B2) were prioritized and the mapping quality (STAR alignment score, mapped length) when deciding the duplicate priming reads. To count gene-level priming frequency, various genic regions and sub-genic regions (FIG. 15A) were defined based on Ensembl gene models from R packages EnsDb.Hsapiens.v86 (human) and EnsDb.Mmusculus.v79 (mouse) (Rainer et al., 2019, Bioinformatics., 35, 3151-3153). For CHEX-seq priming site annotation, R package GenomicRanges (Lawrence et al., 2013, PLOS Comput. Biol., 9, e1003118) were used and ChTPseeker G. Yu, L.-G. Wang, Q.-Y. He, ChTPseeker: an R/Bioconductor package for ChTP peak annotation, comparison and visualization. (Yu et al., 2015, Bioinformatics., 31, 2382-2383). The GO enrichment analysis was done with R package clusterProfiler (Chen et al., 2013, BMC Bioinformatics., 14, 128; Kuleshov et al., 2016, Nucleic Acids Res., 44, W90-W97).

Porphyrin Metalation DNAzyme Homologous ssDNA Loci

Catalytic core sequences of the synthetic DNAzymes previously shown to be active in porphyrin metalation from the database DNAmoreDB (Ponce-Salvatierra et al., 2021, Nucleic Acids Res., 49, D76-D81) were downloaded. They were then aligned to the human and mouse references using BLAST with the criteria (1) 85% mapped length, (2) 0 mismatch and (3) overlapping with at least one CHEX priming site. This led to 326 and 904 porphyrin metalation homologous ssDNA loci being identified in human and mouse, respectively.

### Mesoporphyrin Metalation Assay

All standard reagents and solvents were purchased from commercial sources and used as received. Mesoporphyrin IX (mPIX) was purchased from Frontier Scientific®. UV-vis absorption spectra were recorded on a Lambda 365 UV-Vis spectrophotometer (PerkinElmer®). A quartz micro-cuvette (Starna Cells, 0.1 ml) was used for spectroscopic measurements. All aqueous solutions were prepared using deionized water (dH₂O). SB buffer consisted of Tris (100 mM), NaOAc (200 mM), KOAc (25 mM), Mg(OAc)₂ (10 mM), Triton X (0.5% by weight) and DMSO (5% by volume). pH was adjusted to near-neutral (˜7.2) using NaOH (12N) or HCl (6N). Stock solutions of mPIX in DMSO (15 mM), Pb(OAc)₂ in dH₂O (50 mM) and Zn(OAc)₂ in dH₂O (50 mM) were used to prepare mixtures for kinetic experiments. Kinetics of Zn²⁺ insertion into mPIX was monitored using optical absorption spectroscopy. All experiments were conducted at 23° C. A solution for spectroscopic measurements was prepared directly in a micro-cuvette. The cuvette was charged with SB buffer (100 μL), and all components were added to it by a micro-pipette using the respective stock solutions. The final concentrations were: mPIX (1.5 μM), Pb(OAc)₂ (1 mM), Zn(OAc)₂ (1 mM), DNAzyme (1 μM) or gDNAzyme (25 ng/100 μL). After addition of the last component (typically Zn(OAc)₂), absorption spectra were recorded every 30 min during 24 h. Zn²⁺ insertion was monitored by measuring the absorbance at the maximum of the Soret band (410 nm) corresponding to Zn-mPIX. To verify that the change in the absorbance was indeed due to the formation of the target complex (Zn-mPIX), the Q-band region was examined in the end of each kinetic run, confirming that the absorption in the Q-band region was consistent with that in the Soret region. Each kinetic measurement was repeated three times to ensure reproducibility.

In Vitro gDNAzyme Catalytic Kinetics

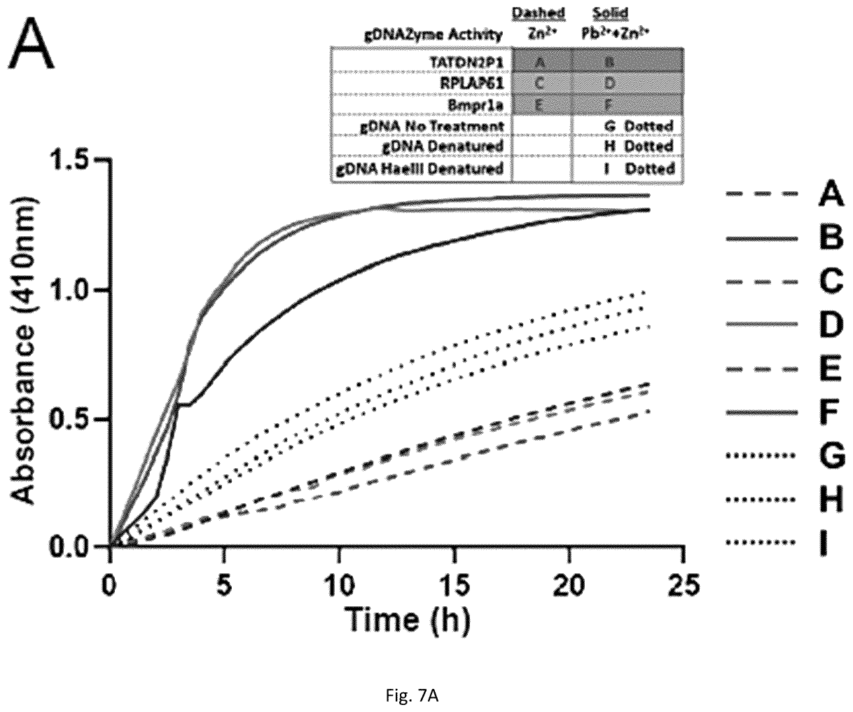

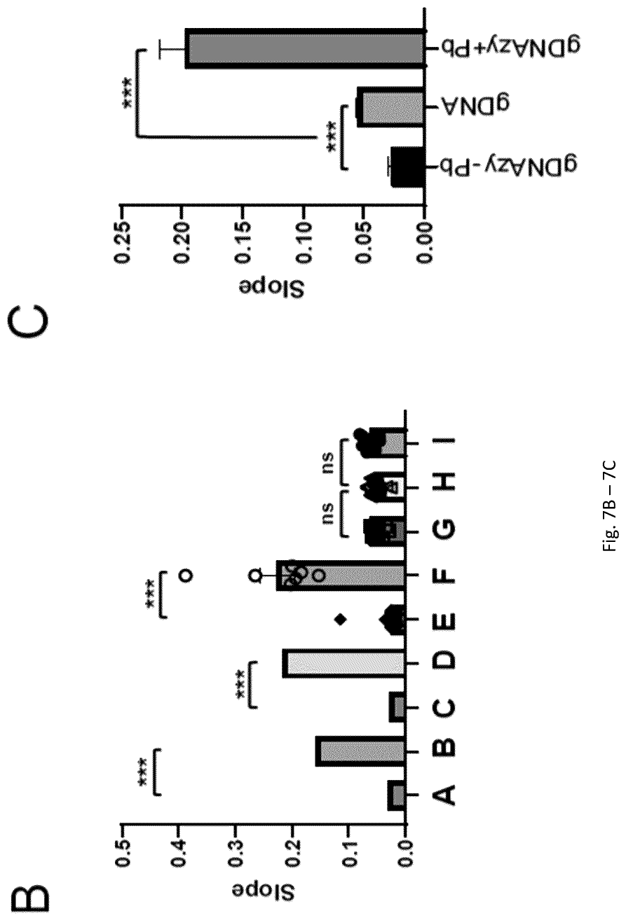

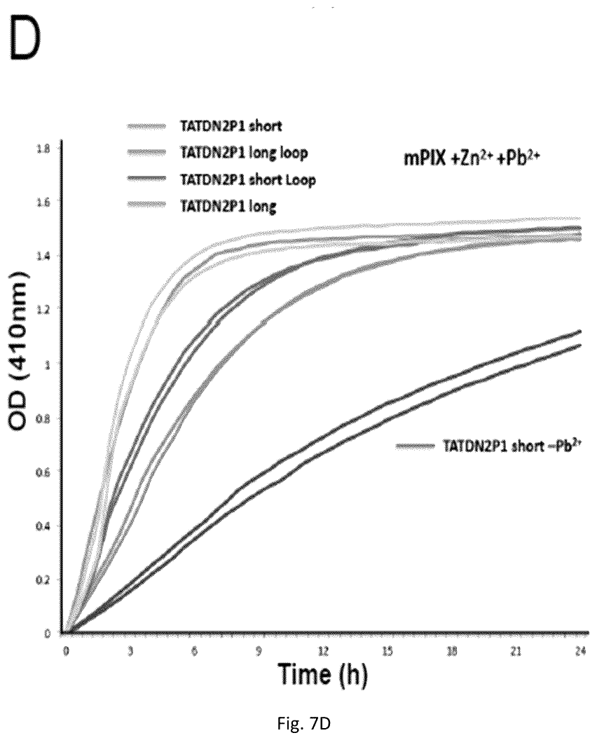

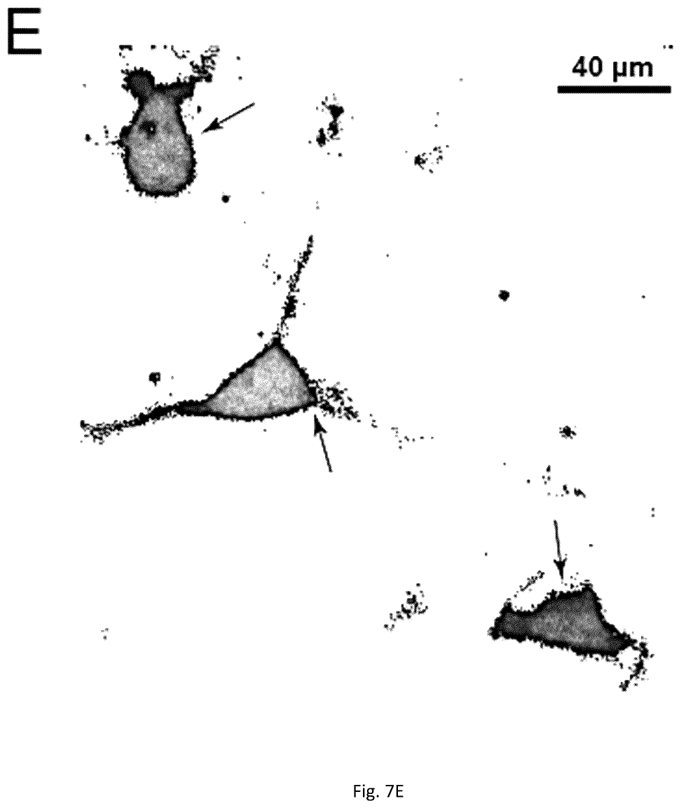

The activity of the enzyme was calculated by normalizing the time course of the measurement (see FIG. 7A, d(P)/dt) to the maximum rate (Vmax) and calculated the time constant of activity (tau) as half the maximum rate for the entire measurement (see FIG. 7B, C). The time constants for each segmented measurement (30-minute measurements over 24 hrs) were also measured and observed a statistical significance for the different DNAzymes (see FIG. 7B, FIG. 7C).

In Vivo Localization and Analysis of gDNAzyme Loci Using FRET-FISH

The FRET-FISH Probes for DNAzyme were designed based on two FISH probes couples to two fluorescent dyes (ATTO550/ATTO590) with overlapping spectra targeting two proximal DNA sequences that close enough to generate FRET that can be detected (Table 2). The labeled probes are 21-25 bases long and are 5 bases away with the ATTO590 facing toward the ATTO550. The proximity of ATTO550 and ATTO590 generates a FRET signal that shows the spatial location of predicted gDNAzymes inside cells. Primary mouse neurons were cultured at 12 mm coverslip (Buchhalter et al., 1991, Brain Res Bull., 26, 333-8). On the day of hybridization, the cells were rinsed in 1×PBS/0.12M sucrose (pH7.4) and fixed with fresh prepared 4% formaldehyde (PFA)/1×PBS/0.12M Sucrose for 30 min at RT. Subsequently, the cells were quenched in 0.1M glycine for 10 min to remove unreacted PFA and washed in 1×PBS (pH7.4) for 5 min 3 times. The cells were permeabilized in 0.1% Triton X-100 for 10 min RT and washed again in 1×PBS (pH7.4) for 5 min 3 times.

Table 2. List of gDNAzyme Analysis Sequences. The first set of sequences for genomic DNA sequences that were tested for DNAzyme activity. The second set of sequences are the FishFRET probes for Bmpr1a.

| | SEQ ID | |
|---|---|---|
| Primer Name | NO | Sequence |
| TATDN2P1long | 27 | CAAAGAGCAGTCTATCTAGGCTGGTGTTTTCCACAAAATAGGGTGGGTGGGTGGGTTTT |
| | | CTTTCACTCCCTTCACCATCTATCCTAATGTT |
| TATDN2P1short | 28 | GTTTTCCACAAAATAGGGTGGGTGGGTGGGTTTTCTTTCACTCCCTTCA |
| opTATDN2P1anneal- | 29 | AACATTAGGATAGATGGTGAAAAACACCAGCCTAGATAGACTGCTCTTTG |
| short-loop | | |
| opTATDN2P1anneal- | 30 | AACATTAGGATAGACTGCTCTTTG |
| long-loop | | |
| Bmpr1a | 31 | TAATAATGGATTGGGTGGGTGGGTGGGTACAAGGATGGAGATGA |
| RPL7AP61 | 32 | GTTTGATGGAAAAATGGGTGGGTGGGTGGGTAGATGAATGGATAGAT |
| Bmpr1aFishFRETProbes-3′ | 33 | ccaatccattaaaattactct/3ATTO550N/ |
| Bmpr1aFishFRETProbes-5′ | 34 | /5ATTO590N/cccttttagacctaaaaggtg |

The FISH hybridization mix was prepared by mixing the FRET oligonucleotide probe pairs in hybridization buffer (4×SSC, 0.5 mM EDTA, 10% formamide) at final concentration 100-200 uM, and added to each cell for overnight reaction at RT. The next day, the cells were washed sequentially with 4×SSC for 5 min 2 times, 2×SSC for 5 min 2 times, 0.5×SSC for 5 min 3 times, and distilled water once. The cells mounted on glass slide in Vector mounting media containing DAPI. The cells were screened for FRET detection using a Zeiss 880 confocal microscope. To detect low-level fluorescence signals from FRET with high confidence, the emission signals were separated by spectrum using a grating. Both acceptor and emission signals were spectrally separated in 15 nm resolutions, divided into four channels each, and integrated using a multichannel GaAsP PMT. In contrast to conventional FRET quantification using two channels, four channels for both donor and acceptor emission signals were used to evaluate FRET efficiency. The mean fluorescence signal of each channel in the cell of interest was calculated and compared to the fluorescence signal changes of each pixel. Any increase or decrease in the signal that was more than defined Z standard deviations from the mean value of every pixel fluorescence value in the cell of interest was considered significant. To determine successful FRET in single pixels, the value of each channel was computed and all four channels in the donor or acceptor had to show either a decrease or increase respectively. This process not only increased the detection sensitivity of FRET at the level of single pixels but also significantly decreased the likelihood of false positives due to unavoidable system noise.

### Data and Materials Availability

Raw FASTQ data, per-sample priming sites (in BED format) and gene-by-sample priming count matrices are deposited to GEO under accession number GSEXXXXX CHEX-seq NGS pipeline is freely available at github_com/kimpenn/chex-seq. Data analysis scripts are public at github_com/kimpenn/chex-analysis.

The results of the experiments are now described.

### Overview of CHEX-Seq Analysis and Experiment Design

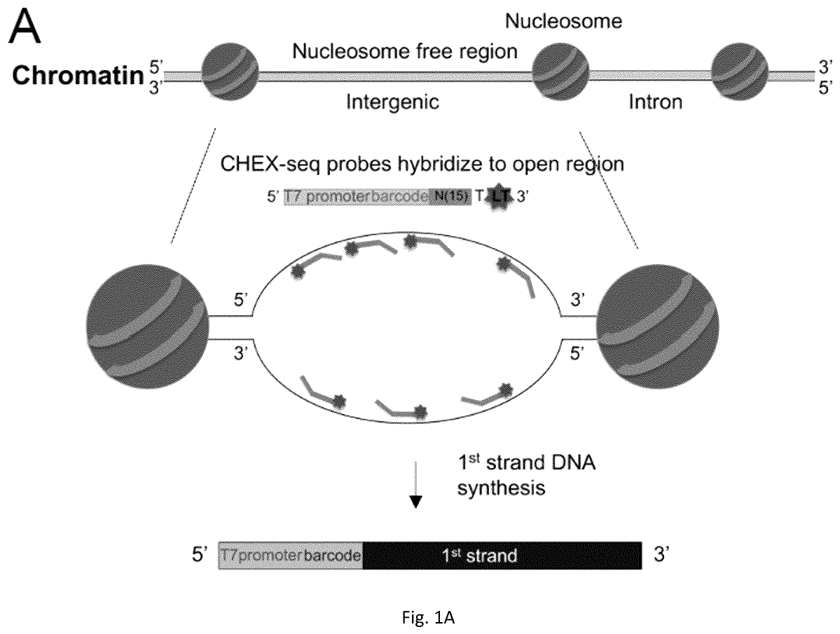

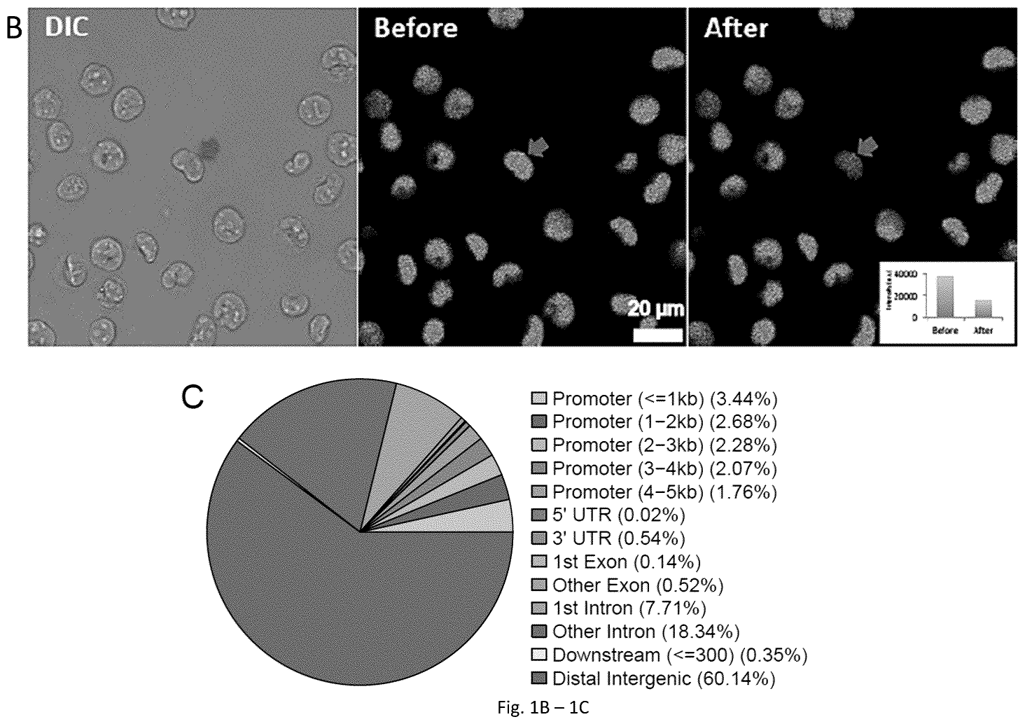

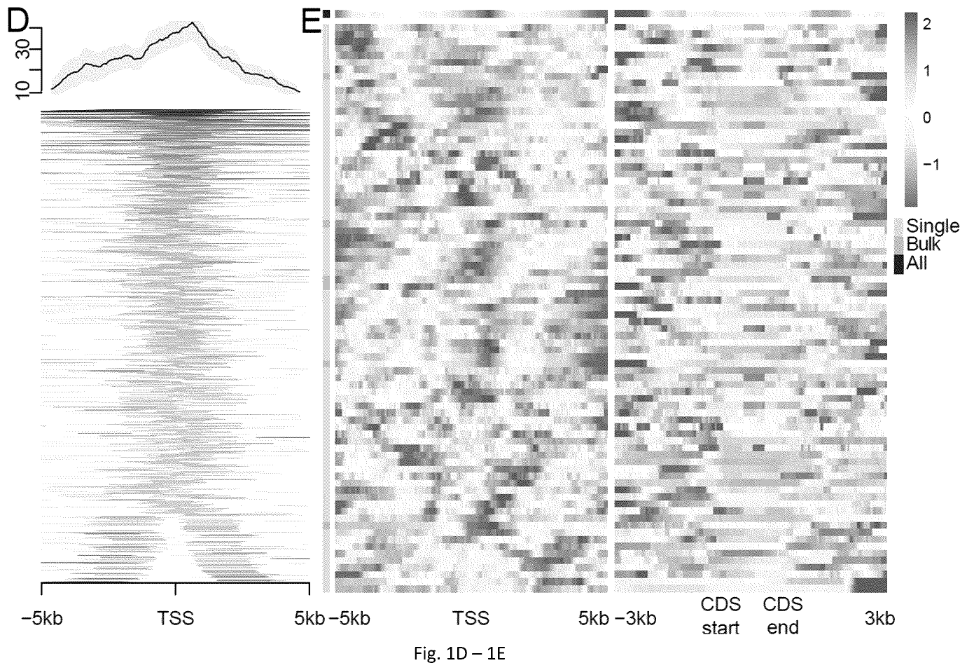

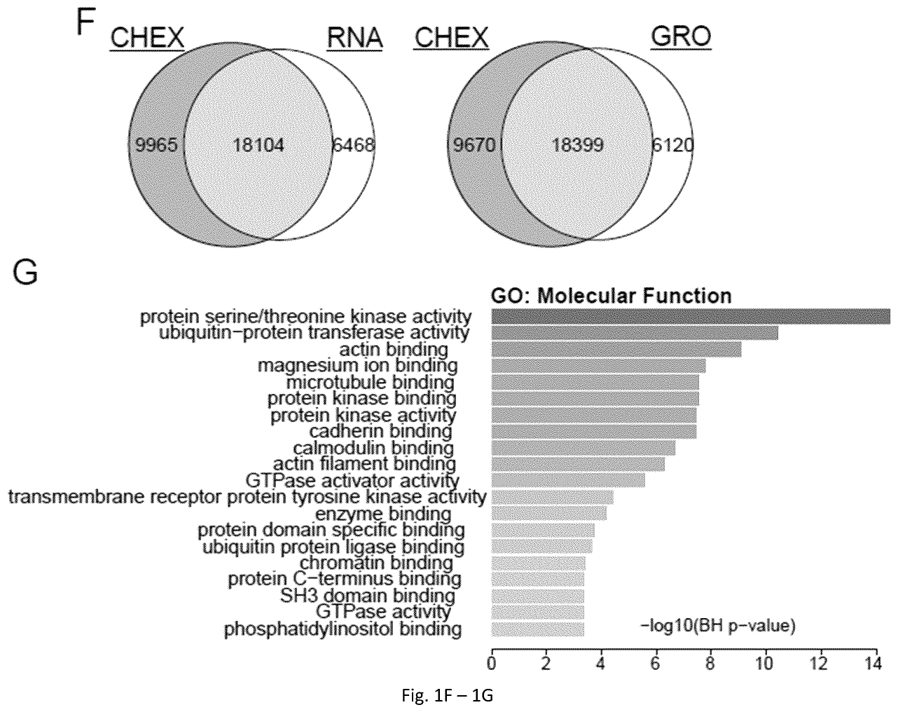

To assay ssDNA at single-cell resolution in situ, a ssDNA chromatin interrogator as a multifunctional oligonucleotide probe was designed. It is composed of three parts: the 5′ barcode, the degenerate sequence, and the 3′ “lightning terminator” (FIG. 1A and FIG. 8). The barcode can distinguish multiplexed samples from the same library. The degenerate sequence is designed to anneal to single-stranded gDNA in fixed cells (FIG. 8, Table 1). The lightning terminator is a fluorescently tagged, photo-reversibly blocked deoxynucleotide. It will remain inactive at the site of annealing until light activation, which liberates a free 3′-OH that serves as a primer for spatially-localized, polymerase-mediated in situ cDNA synthesis (Tecott et al., 1988, Science, 240, 1661-4; Eberwine et al., 1992, Methods Enzym., 216, 80-100) (FIG. 1A). After in situ DNA synthesis, the cDNA was removed with 0.1N NaOH, copied into dsDNA, and linearly amplified using the T7 RNA polymerase (a.k.a. “aRNA amplification”) (Van Gelder et al., 1990, Proc Natl Acad Sci U A., 87, 1663-7; Eberwine et al., 1992, Proc Natl Acad Sci U A. 89, 3010-4). The aRNA product was subsequently reverse transcribed to 1^{st} and 2^{nd} strand DNA with custom primers, PCR amplified, made into libraries, and sequenced using 75 bp paired-end reads (FIG. 9). Raw reads were filtered based on the barcode/primer quality (FIG. 10), aligned to the respective (human or mouse) reference genome, further filtered by the alignment quality, and finally the 5′ end of the barcode-carrying reads was taken as the ssDNA site. It is noteworthy that, unlike other open-chromatin assays (ATAC-, DNase-, FAIRE-seq), the barcoded design and strand specific prep enable CHEX-seq to preserve ssDNA's strand information.

To show the utility of CHEX-seq, extensive tests were run on distinct species, cell types as well as experimental conditions. They include: two species (human and mouse), two classes of brain cells (astrocytes and neurons), two types of neuronal cell preparations (dispersed primary culture and in situ tissue section), and finally, K562 cells perturbed by TPA (12-O-Tetradecanoylphorbol-13-Acetate), a protein kinase C (PKC) activator and peripheral blood lymphocyte mitogen (Niedel et al., 1983, Proc. Natl. Acad. Sci. U.S.A, 80, 36-40; Nagel et al., 1982, Clin. Exp. Immunol., 49, 217-224). which can induce chromatin accessibility changes in K562. The wide range of applications proves a general utility of the methodology, and permits interesting biology correlating ssDNA to genome maintenance, replication, and transcriptional activity to be uncovered.

### CHEX-Seq Benchmark in Human K562 Cells

Human K562 cells were chosen by ENCODE for extensive chromatin analyses (The ENCODE Project Consortium, 2012, Nature, 489, 57-7; Davis et al., 2018, Nucleic Acids Res., 46, D794-D80); hence this cell line was selected for benchmark. After fixation, K562 cells were gravity deposited onto poly-L-lysine-coated cover slips, then permeabilized and washed in PBS. Annealing of the CHEX-seq probes to the fixed cells shows the tagged fluorescence concentrating in the nucleus of the cell (FIG. 1B, middle). The lightning terminator was illuminated by 405 nm (UV) laser at 60% power and 30 s per pixel, whereupon a 45˜80% decrease in the fluorescence was observed (FIG. 1B, right). This indicates the loss of the fluorescent moiety and the freeing of a 3′-hydroxyl group that can later prime DNA synthesis.

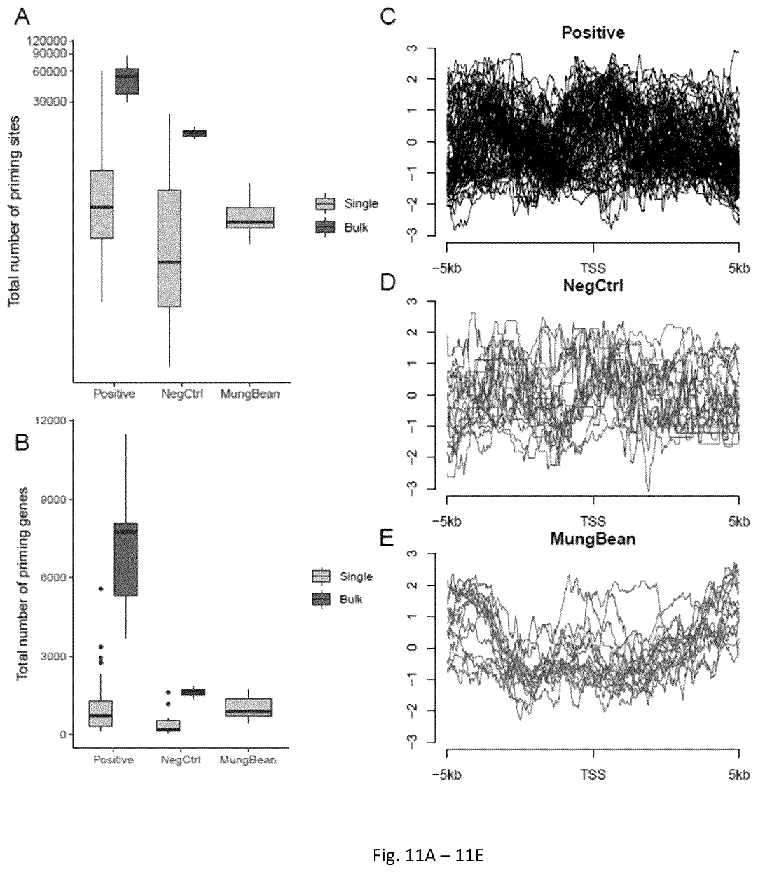

After a series of QC filtering (for details see Methods: Data Processing), the total number of priming sites in each non-control K562 sample varies from 305 to 60,437 (median=2,640) for single cells, and from 30,118 to 85,382 (median=53,357) for multi-cell (bulk) samples (FIG. 11). As a type of negative control, 14 K562 cells were treated with mung bean nuclease to digest ssDNA, and experiments showed significant reduction in the total number of priming sites, ranging from 1,139 to 4,632 (median=1,890). The greatest reduction happened to the other type of negative control where there were no CHEX-seq probes or no light activation: only a median of 748 or 14,704 total priming sites observed in single cells or bulk samples, respectively (FIG. 11). In conclusion, CHEX-seq has a meaningful specificity in ssDNA detection despite a relatively high background in bulk controls.

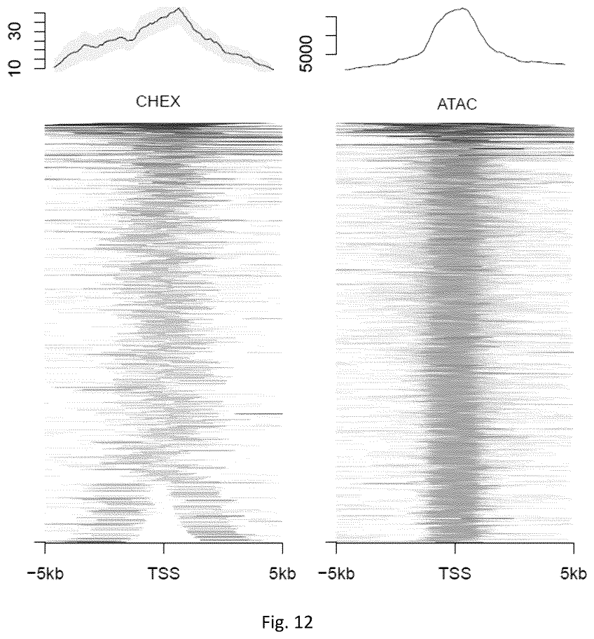

The percentage of CHEX-seq priming sites that map to the gene body, the flanking regions, including the 5′ promoter, TSS, exons, introns, the 3′-proximal area, and distal intergenic regions were computed (FIG. 1C). K562 cells show the highest proportion of priming sites in intergenic regions (>60.1%), followed by introns (˜26.1%), and then by promoter regions (less than 5 kb to the TSS) (˜12.2%). Further breaking down the TSS 5 kb neighborhood into the proximal (<1 kb) and the distal (4-5 kb) regions, experiments showed almost two times more priming sites in proximal regions than distal regions (1.8%), consistent with the notion that chromatin tends to be more accessible near the TSS. Indeed, experiments showed TSS enrichment in most non-control samples, while weak or no enrichment in negative controls (FIG. 11B). Combining the coverage across all non-control samples created a distinct peak centered at the TSS (FIG. 1D), resembling the TSS peak observed in ATAC-seq (Buenrostro et al., 2013, Nat Methods, 10, 1213-8). However, closer comparison revealed a distinction between the two assays: ATAC-seq has a sharp peak around the TSS, while the CHEX-seq has a wider peak with an extended slope 5′ to the TSS (FIG. 12A). These findings suggest that there is an anticipatory single-strandedness to the TSS of genes that are likely to be transcribed (Swamynathan et al., 1998, FASEB J., 12, 515-522). Alternatively, these CHEX-seq sites found 5′ of the TSS may result from TSS-proximal DNA being uncoiled as a result of the “bursting transcriptional activity” (Suter et al., 2011, Science, 332, 472-474) at the TSS. FIG. 1E shows CHEX-seq coverage from single-cell, bulk, and sample aggregates, pooling annotated features (gene or coding sequence [CDS]): the left panel shows cell-cell variability in CHEX-seq priming locations near the TSS, and the right panel shows an increased propensity for ssDNA near the start of the CDS.

To assess how many of the K562 priming sites correspond to expressed mRNA, the CHEX-seq data were compared with published K562 transcriptome datasets (FIG. 1F). About 73.6% (18,104) of the highly expressed (>median) genes measured by RNA-seq had corresponding CHEX-seq priming. Even with this relatively large overlap, there were still 35.5% (9,965) single-stranded genes that were not highly expressed. Further, it was reasoned that transcriptionally associated ssDNA might be more correlated to ongoing transcription and nascent mRNAs, which could be detected by GRO-seq, a real-time transcription runoff assay (Danko et al., 2015, Nat Methods, 12, 433-8). Therefore, CHEX-seq were compared to GRO-seq transcripts (Lladser et al., 2017, J Math Biol., 74, 77-97). and observed similar but more overlapping genes (˜75.0%) with a slight decrease in the CHEX-seq unique genes (˜34.5%). Gene Ontology (GO) (Ashburner et al., 2000 Nat Genet. 25, 25-29) enrichment analysis of the CHEX-RNA overlapping genes identified cell signaling, kinase activity, and GTPase regulatory pathways as significantly enriched, consistent with the fact that K562 cells are a transformed cell line (FIG. 1G).

### CHEX-Seq Compared to Other Open-Chromatin Assays

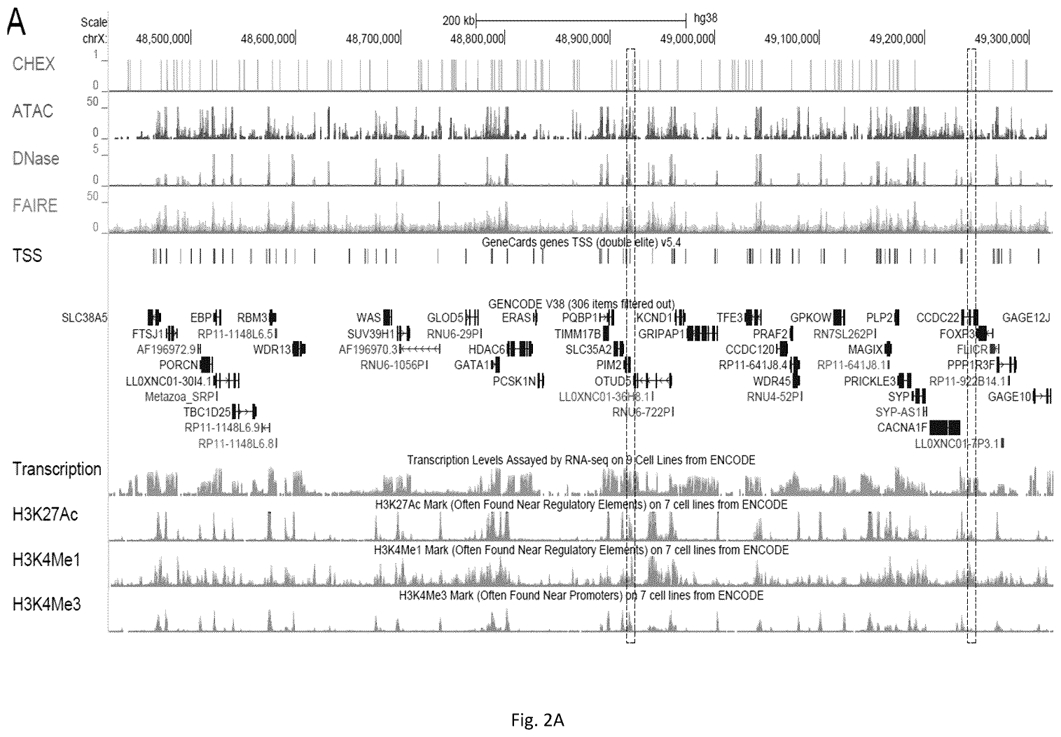

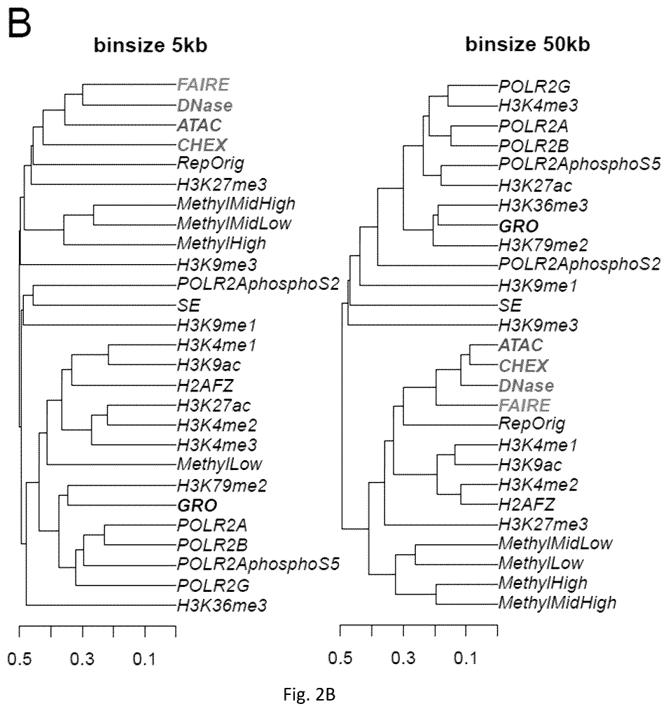

Having assessed CHEX-seq's TSS propensity and transcriptional association, CHEX-seq were further benchmarked against three other well established chromatin assays (ATAC-, DNase-, FAIRE-seq) in genome-wide coverage. FIG. 2A exemplifies the comparison in an 800 kb region of Chromosome X (chrX:48,500,000-49,300,000) from the UCSC Genome Browser (Kent et al., 2002, Genome Res. 12, 996-1006; Lee et al., 2022, Nucleic Acids Res. 50, D1115-D1122 (2022). There is commonality between CHEX-seq and the other three methods, however, regions unique to each method were noted. For example, the first dashed-line box highlights the gene OTUD5, whose 3′ UTR and downstream area are shared by ATAC-seq and CHEX-seq, while the gene PIM2's intron is shared by CHEX-seq, DNase-seq and FAIRE-seq; the second dashed-line box highlights the sixth intron of CCDC22 where all the four methods overlap, though CHEX-seq appears slightly downstream the other three (FIG. 2A). It was reasoned that the discrepancy could be explained by the fact that different epigenomic assays have different genomic scales due to both the biological nature of the signals detected by each technique and the chemistry of each assay.

To better quantify the genome-wide relationship between different open-chromatin assays, signal concordance was computed between CHEX-, ATAC-, DNase- and FAIRE-seq, against a select set of K562 epigenomes (broad and narrow histone modifications, DNA methylome, Pol II ChIP-seq, GRO-seq, super-enhancers, and replication origins) in fixed-sized bins followed by hierarchical clustering (FIG. 2B). At the size scale of 5 kb windows, the four chromatin methods cluster together while CHEX-seq appears the most distant (FIG. 2B, left). The GRO-seq transcriptome (in bold) is even more distant, indicating that there is not a one-to-one overlap between detectable open chromatin and newly transcribed genes, even with the pronounced overlap between CHEX-seq and highly transcribed nascent mRNAs as shown in FIG. 1F. At the size scale of 50 kb, again, FAIRE-, ATAC- and DNase-seq form a cluster with CHEX-seq, with CHEX-seq displaying the closest relationship to ATAC-seq (FIG. 2B, right). As the average size of a human gene is ˜42 kb and the functional transcriptional chromatin unit is ˜50 kb (Hegedus et al., 2018, Nucleic Acids Res., 46, 10649-10668), these data suggest that many of the same open-chromatin associated genes are identified with each of these procedures, but the ssDNA identified by CHEX-seq is distinct from the dsDNA open chromatin identified by the other approaches. A direct overlap would not be expected, as the other procedures have a target bias for dsDNA (ATAC- and FAIRE-seq) or are indiscriminate of single or double strands (DNase-seq) as compared to CHEX-seq's ssDNA specificity. Moreover, CHEX-seq data are sparser due to fewer numbers of analyzed cells. These results highlight that both double-stranded and single-stranded DNA exist within the open chromatin of genic and intergenic genomic areas, both of which sculpt the open-chromatin landscape of a cell.

In addition to the clustering analysis comparing CHEX-seq with the select set of K562 epigenomes (FIG. 2A, B), this analysis was extended to a full set of 284 K562 epigenomes curated from ENCODE ChIP-seq (Consortium et al., 2004, Science, 306, 636-40), Non-B-form DNA database (Cer et al., 2013, Nucleic Acids Res., 41, D94-D100), and R-loop DRIP-seq (Sanz et al., 2013, Mol. Cell. 63, 167-178), and asked how CHEX-seq differs from the other three open-chromatin assays in the epigenome. Genomic association scores (Heger et al., 2013, Bioinformatics. 29, 2046-2048) for each of the four chromatin assays to each epigenome, then normalized the enrichment scores to rank quantiles to alleviate assay specific confounding factors (such as different coverages or peak breadths), and finally clustered the four assays based on the normalized enrichment scores (FIG. 2C). As highlighted in the zoom-in view of FIG. 2C, CHEX-seq exhibits strong overlap with a unique set of transcription factors (TFs), including the Y-box family (YBX1/3), the mini-chromosome maintenance family (MCM2/3/5/7), non-B-form DNA (direct repeats), and heterogeneous nuclear ribonucleoproteins [hnRNPs] (HNRNPH1, HNRNPUL1, HNRNPK). The Y-box and the MCM protein family have been well known as evolutionarily conserved ssDNA binding proteins that participate in DNA replication and repair (Eliseeva et al., 2011, Biochem. Biokhimiia., 76, 1402-1433; Ashton et al., 2013, BMC Mol. Biol., 14, 9). Interestingly, both protein families are related to transcription as well. For example, YBX1 is reported for the involvement in pre-mRNA transcription, splicing, mRNA packaging and stability regulation (Lyabin et al., 2014, RNA., 5, 95-110) and similarly, MCM2-7 for being necessary for RNA Pol II mediated transcription (Snyder et al., 2009, J. Biol. Chem., 284, 13466-13472). Some members of the hnRNPs are known to bind to single-stranded telomeric DNA in vitro (Huang et al., 2010, Cell Res., 1803, 1164-1174), hence the enrichment in hnRNPs were less surprising. In fact, TFs with DNA/RNA dual binding capacities have been shown previously (Cassiday and Maher III 2002).

More intriguingly, experiments found that DNMT1, one DNA methyltransferase that is responsible for conferring the template strand's methylated status to the newly synthesized strand during replication (Li et al., 1992, Cell, 69, 915-926), ranked much higher for its enrichment in CHEX-seq (18th of 284) than ATAC- (152nd of 284), DNase- (221st of 284) or FAIRE-seq (147th of 284) (FIG. 2C). Although CHEX showed higher enrichment in low-/un-methylated genome (MethyLow) than other three assays (FIG. 2C), it suggests a possible role of ssDNA in DNA methylome maintenance, which is also a single-stranded process. Besides the enrichments, experiments also showed some epigenomes are exclusively depleted in CHEX-seq. They are mostly TFs, and many participate in development, proliferation, or neoplasia, e.g., GATA1, JUNB, JUND, MYC, etc. (FIG. 2C, upper brace). These sites could represent genes whose expression is limited to a particular stage of development or disease state (oncogenesis) which is reflected in their single strand/double strand ratios.

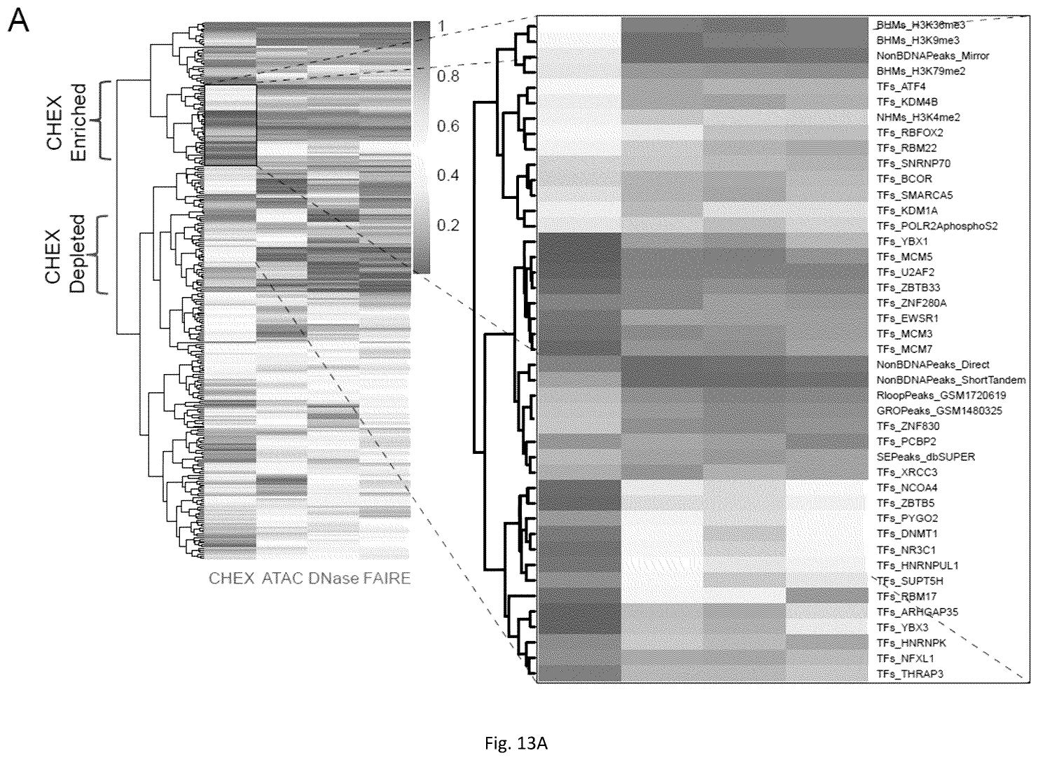

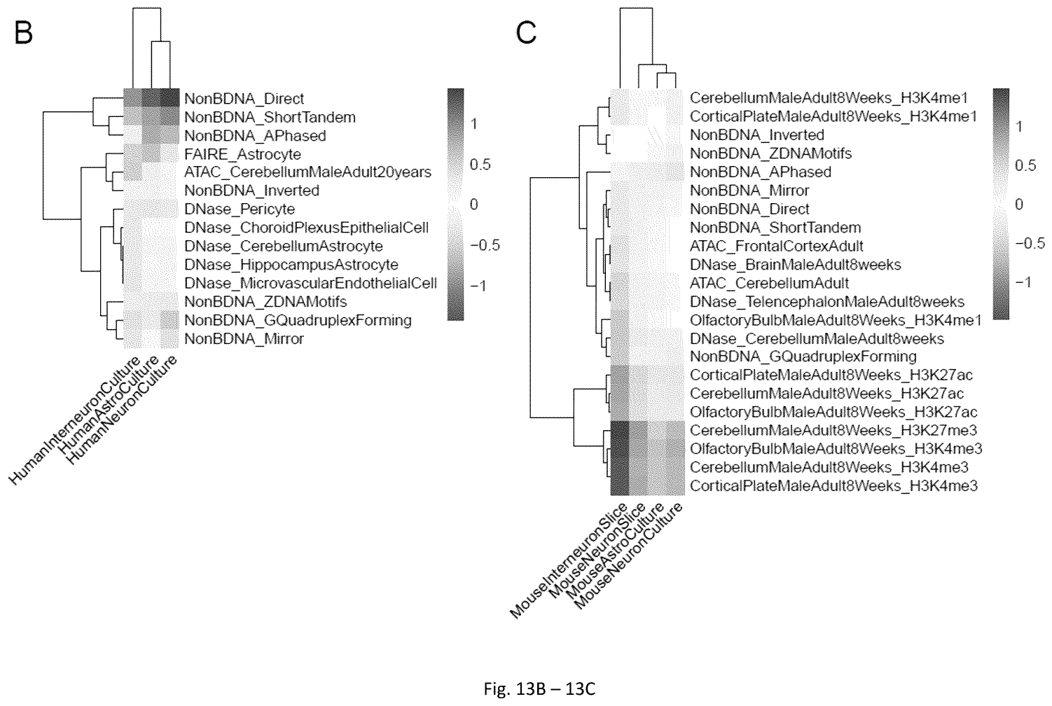

To reduce spurious reads, the barcode/primer class criterion were tightened (quality level from A/B/C to A/B only) and the alignment criterion (minimal mapped length from 20 bp to 30 bp), then repeated this analysis. This time experiments not only recapitulated the enrichments (YBX1/3, MCM3/5/7, HNRNPK, DNMT1), but also uncovered additional CHEX-seq exclusive epigenomes: R-loops, super-enhancers, and transcriptional activity indicators (POLR2AphosphoS2, GRO-seq), and other types of non-B DNA (short tandem and mirror repeats) (FIG. 13A). In summary, the benchmark results showed ample evidence for CHEX-seq's ability to detect ssDNA in the process of expression, replication and possibly DNA methylation maintenance.

FISH Validation of CHEX-Seq Identified Intergenic ssDNA Loci

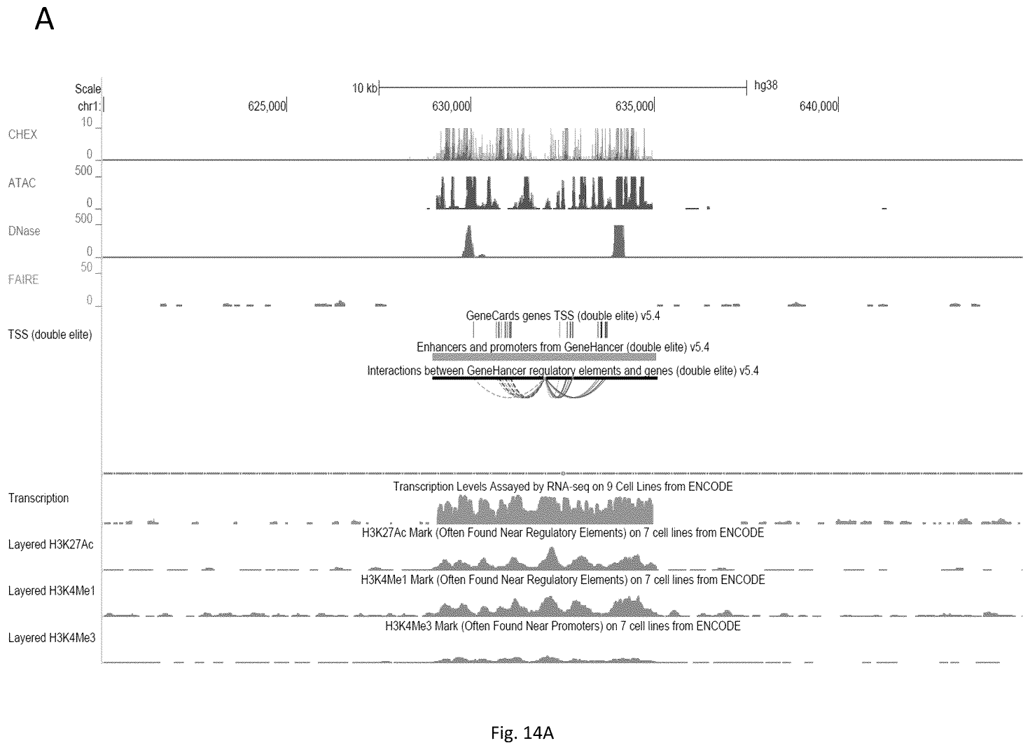

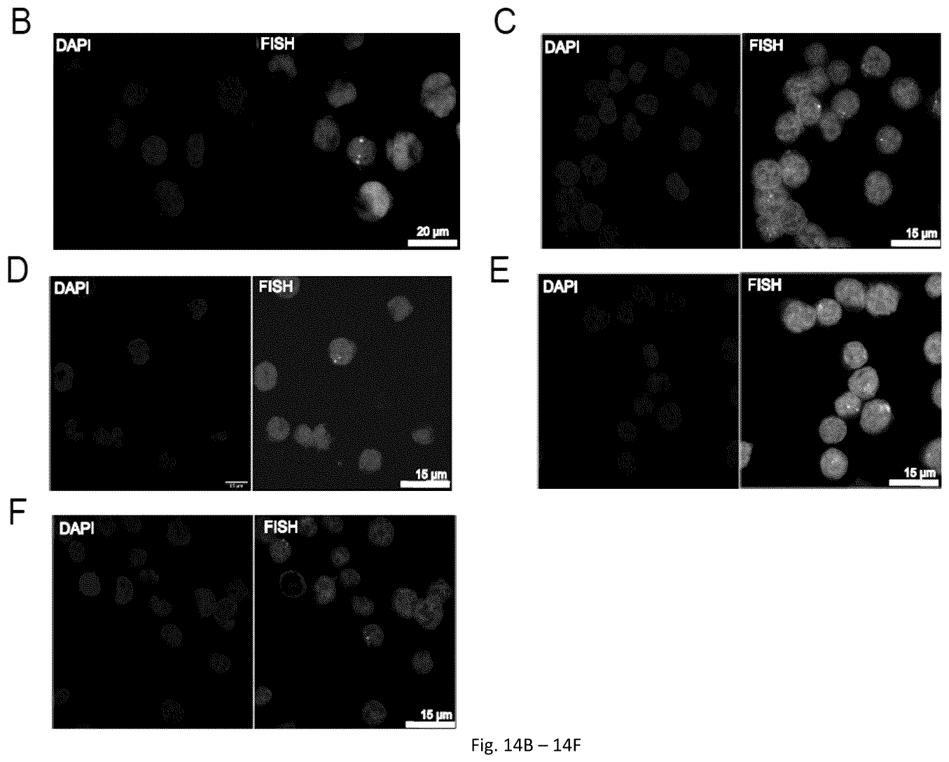

To validate the CHEX-seq predicted ssDNA loci, single molecule FISH (smFISH) was performed for a CHEX-seq priming hotspot on Chromosome 1 (chr1:630737-633960), where ATAC-seq predicted open while DNase-seq predicted limited openness and FAIRE-seq predicted closed (FIG. 14A). Eight 20-mer oligonucleotide probes were synthesized to target this area and these probes were labeled at the 5′-end with the ATTO 590 fluorophore. It is important to note that, as experiments were assessing endogenous single-strandedness, no heat denaturation of the tissue was performed. Therefore, a positive signal could only arise from endogenous single-stranded chromatin. Three strong positive spots are observed in a single nucleus with smFISH (FIG. 14B). This trisomy signal is due to the complicated K562 cell karyotype where some cells have 3 copies of Chromosome 1 (Gribble et al., 2000, Cancer Genet Cytogenet., 118(1):1-8). To further test CHEX-seq and to rule out the interference of RNA transcripts during probe hybridization, a second run of smFISH experiments were performed on other four additional loci from distal intergenic regions of Chromosome 4, 8, 11 and X. All showed at least one positive cell out of ˜10 cells in the field of view (FIG. 14C-F). Specifically, loci chr4:1466001-1468000 (FIG. 14C) and chr11:123002001-123004000 (FIG. 14E) showed ˜30% cells with two or more fluorescent spots. In conclusion, the FISH experiment corroborated that single-stranded chromatin structure that is detected by CHEX-seq.

The Role of ssDNA in Active Transcription and the CHEX-Seq Strand Specific Model

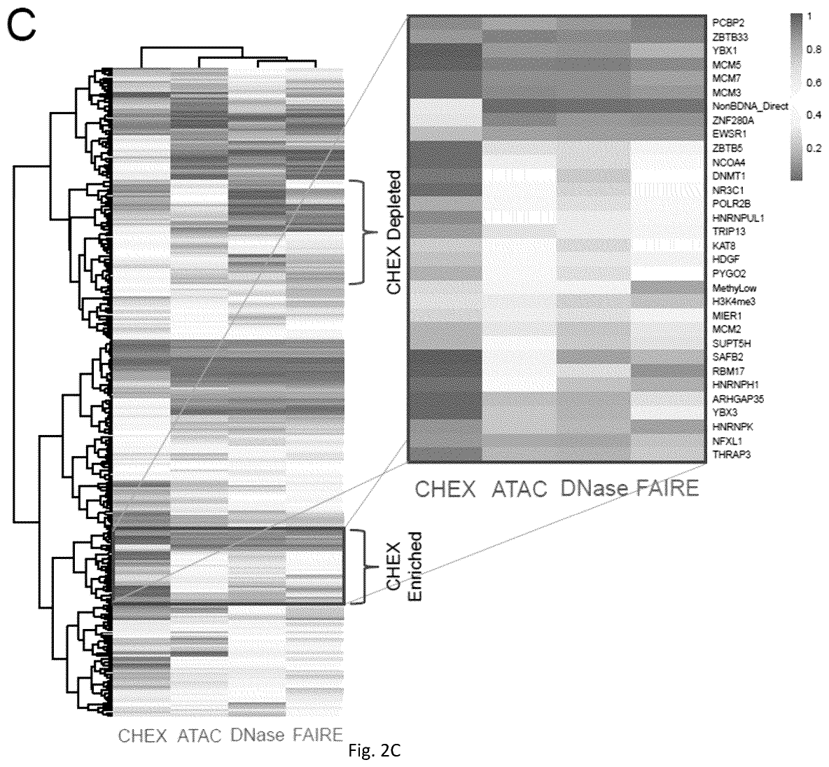

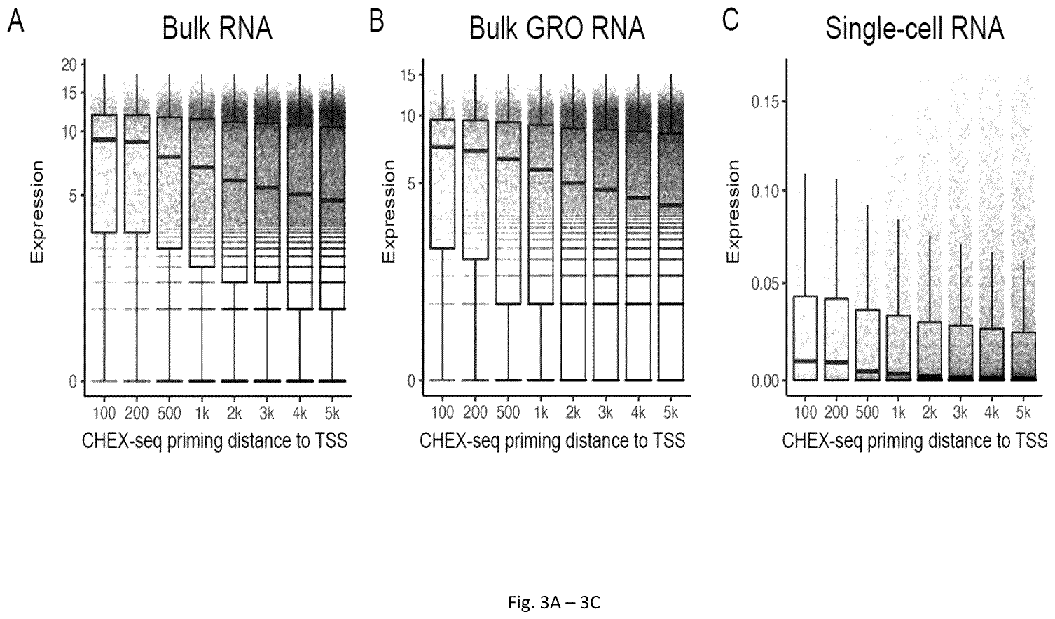

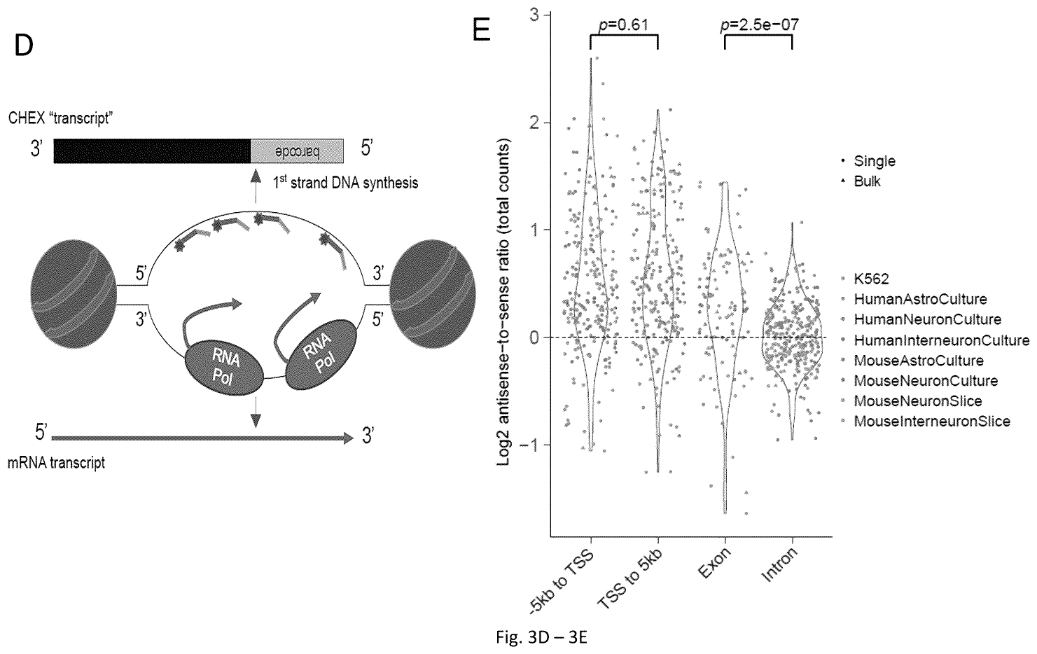

Having established CHEX-seq as a ssDNA detection method, experiments went on to explore how single-stranded chromatin is associated with transcription in a more quantitative way. Genes in K562 were stratified according to their CHEX-seq priming location (if any) with respect to the distance to the TSS, then correlated it to the same gene's mRNA expression level from three different sources—bulk RNA-seq (Lan et al., 2012, Nucleic Acids Res., 40, 7690-7704) bulk GRO-seq (Core et al., 2014, Nat. Genet., 46, 1311-1320) and single-cell RNA-seq [scRNA-seq] (Dixit et al., 2016, Cell, 167, 1853-1866.e17). Experiments showed an anticorrelation between the CHEX-seq priming distance to the TSS and the gene expression level for all three datasets: the closer the CHEX-seq priming sites to the TSS, the higher the median expression level of the corresponding mRNAs (FIG. 3A-FIG. 3C). The same pattern was also found in human and mouse brain cells (FIG. 15A-FIG. 15C and FIG. 15D-FIG. 15G, respectively). These results suggest a regulated plasticity with regard to ssDNA within a gene: i.e., as the transcription machinery moves along the length of the gene, the 5′-open site becomes unavailable for hybridization, perhaps due to reannealing of the single-stranded region or formation of an intrastrand DNA secondary structure (Szlachta et al., 2018, Genome Biol., 19, 89). This suggests that CHEX-seq priming sites have varying half-lives, and those that are proximal to the TSS remain single-stranded for a longer time and correspond to high levels of transcription, thus CHEX-seq priming closer to the TSS is predictive of highly transcribed genes. It was noted that these are not simply due to the differences in RNA stability, as GRO-seq detects newly synthesized nuclear RNA but shows the same anticorrelation (FIG. 3B). Experiments suggest a location dependent model of ssDNA: TSS-proximal ssDNA is associated with ongoing gene expression whose single-stranded accessibility decays with 5′ to 3′ progress of transcription, while distal ssDNA is more likely related to other conformational regulation of the DNA such as genome replication or methylome maintenance.

Unlike other open-chromatin assays such as ATAC-, Dnase- or FAIRE-seq, CHEX-seq identifies the DNA strand that is being copied as its primer-extension in situ transcription method informs sequence directionality. Since the RNA Pol II transcriptional complex binds to the DNA and synthesizes mRNA transcripts in a 5′ to 3′ direction by transcribing the antisense (template) strand, it was hypothesized that CHEX-seq probes might be preferentially bound to the potentially more accessible sense strand, giving rise to an excess of antisense-strand “CHEX-seq transcripts” (FIG. 3D). The ratio of antisense to sense-strand reads were calculated for different regions of the annotated gene model: TSS 5 kb upstream, TSS 5 kb downstream, exons and the introns. As expected, experiments found generally more antisense-stranded reads in the transcribed regions of the genome, and the strongest antisense bias was in the TSS 5 kb upstream, followed by the TSS 5 kb downstream, then by the exonic region (FIG. 3E). In contrast, the intronic region showed the least bias (FIG. 3E). It was reasoned that the strongest antisense bias in TSS proximity to be due to promoter-proximal RNA Pol II pausing as a pervasive transcription regulatory mechanism in metazoans (Adelman et al., 2012, Nat. Rev. Genet. 13, 720-731; Schier et al., 2020, Genes Dev., 34, 465-488). It can be speculated that the disappearance of the bias from exons to introns was a result of the uneven distribution of RNA Pol II along the gene. As observed in mouse embryotic stem cells (mESCs), RNA Pol II has peak density at the TSS, then rapid decay after the TSS, with a gradual rebound towards the termination site (Grosso et al., 2012, Genome Res., 22, 1447-1456). Therefore, the antisense strand of the first and the last exon are hypothesized to show greater RNA Pol II occupancy than other exons or introns. Herein, CHEX-seq's TSS-proximal signal was established as an indicator of active transcription, and further proposed and tested the model for CHEX-seq strand bias due to polymerase/TF occupancy preference in transcribed regions.

Dynamic Regulation of ssDNA Upon Chromatin Perturbation in K562 Cells

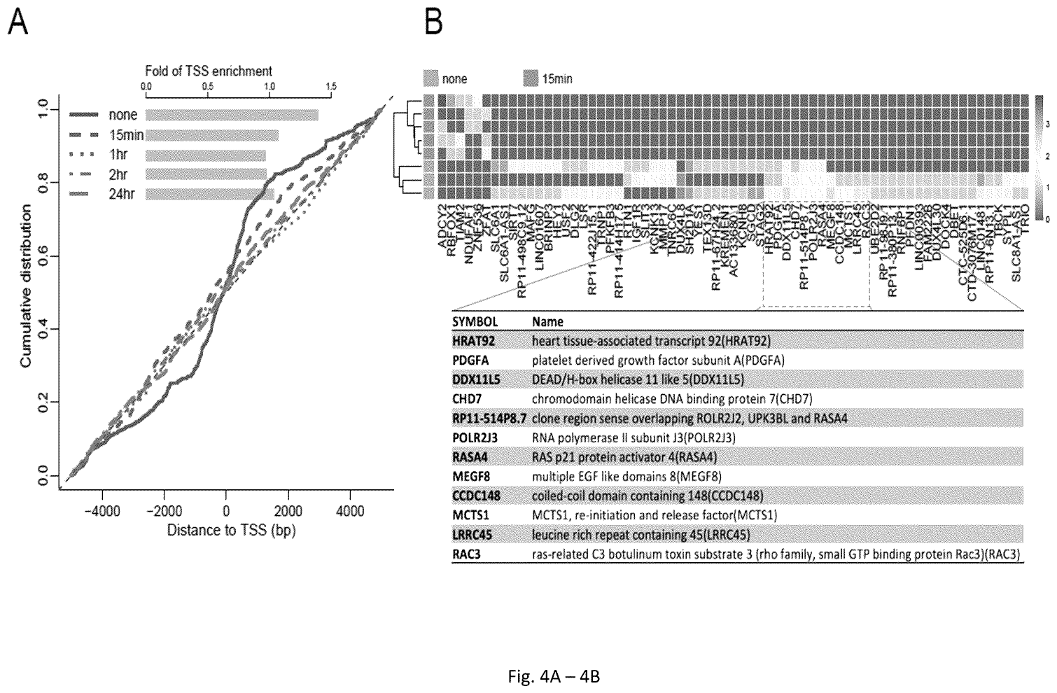

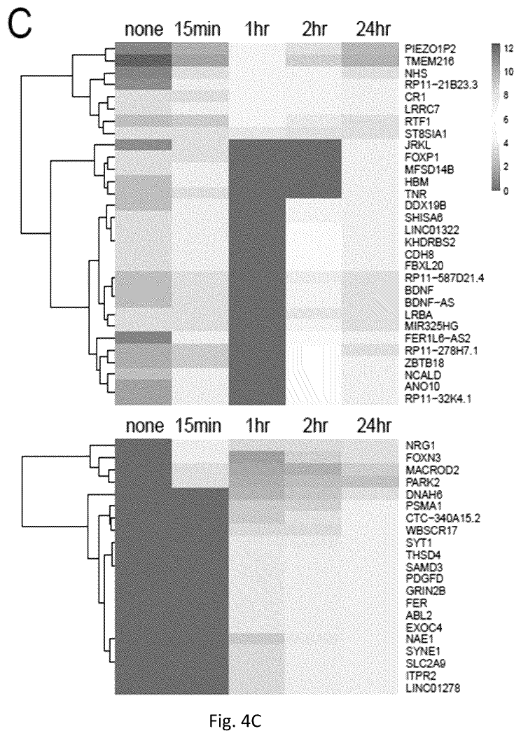

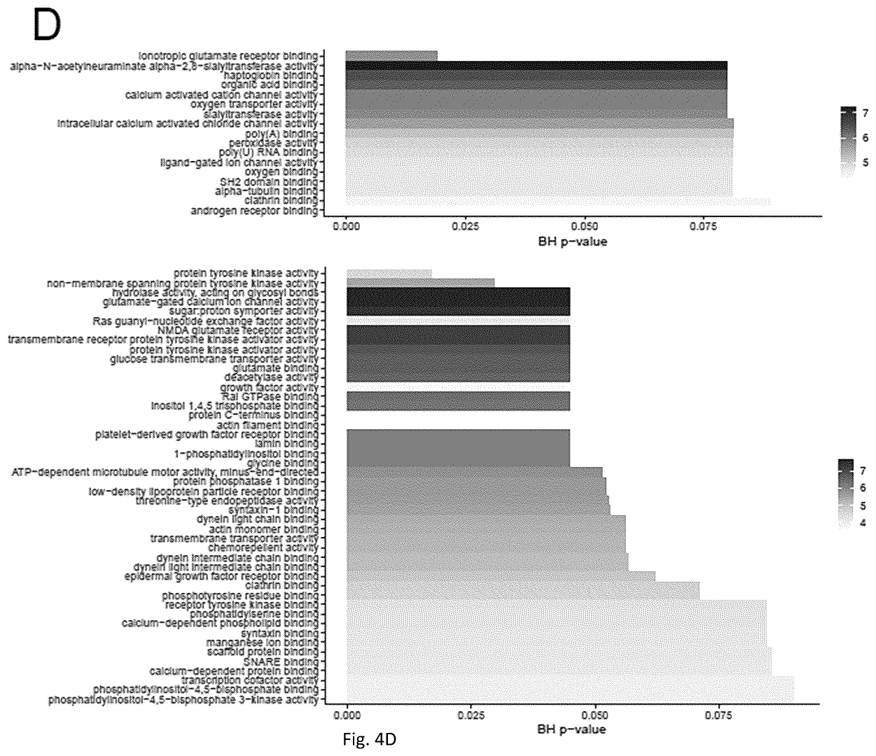

Given the ability of CHEX-seq to identify single-stranded open chromatin, experiments were designed to investigate if these regions are dynamically regulated. To address this, K562 cells were treated with TPA, a PKC activator known to modulate K562 gene expression and induce differentiation in leukemic cells (Ezawa et al., 1997, Hematol. Oncol., 15, 151-161; Naour et al., 1997, Leukemia, 11, 1290-1297; Sun et al., 2013, Blood, 122, 1209-1209). To capture the time-dependent changes in ssDNA, the single-cell replicates were aggregated at each time point: pre-treatment, 15 minutes, 1 hour, 2- and 24-hours post-treatment, and plotted the cumulative distribution of ssDNA as a function of the priming distance to the TSS, which was further summarized as the fold of enrichment score at each time point (FIG. 4A). As anticipated, experiments showed that the highest fold of ssDNA enrichment around the TSS in untreated samples, and upon treatment, a steep decrease at 15 minutes after treatment, followed by continued decrease leading to the minimum at 1 hour. After that, the TSS ssDNA enrichment began to recover, and reached increasingly higher level at 2 and 24 hours, though the end-point enrichment still fell short of the level at 15 min. Collectively, this time-dependent trend suggested a rapid and time-dependent regulation of single-stranded open chromatin in K562 cells.

Given that TSS ssDNA enrichment showed the sharpest decrease at 15 minutes, experiments were designed to address what genes or pathways perturbed ssDNA openness compared to the pre-treatment baseline. As shown in FIG. 413, ssDNA regions in 67 genes were found to be highly differentially regulated immediately upon perturbation. Among them 6 genes (ADCY2, RBFOX1, TIAM2, NDUAF1, ZNF536, ZFAT) showed increased ssDNA openness, 61 genes exhibited decreased ssDNA openness. GO enrichment analysis suggested that the genes gained ssDNA openness are enriched in cellular response to forskolin (GO: 1904322), c AMP biosynthetic process (GO:0006171) and activation of protein kinase A activity (GO:0034199) at an adjusted p-value 0.03. The genes that lost ssDNA openness are only significantly (adjusted p-value 0.04) enriched in limb development (GO:0060173). Despite lacking statistical significance, experiments still showed several TPA target relevant genes change in ssDNA architecture (FIG. 4B, table inset). For instance, HRAT92 is a lincRNA specifically expressed in the heart (Fagerberg et al., 2014, Mol. Cell. Proteomics MCP., 13, 397-406) and (11D7 and MEG8 are members of heart morphogenesis (GO:0003007). Consistent with this finding, TPA has been reported to be able to regulate cardiomyogenesis via PKC/ERK signaling in mESCs (Radaszkiewicz et al., 2020, Sci. Rep., 10, 15922. Moreover, MCTS1 and PDGFA are components of regulation of cell population proliferation (GO:0042127), RAC3 is involved in regulation of small GTPase mediated signal transduction (GO:0051056), and RASA4 is involved in negative regulation of GTPase activity (GO:0034260). These findings generally agree with TPA's mitogenic or pro-differentiation effect on blood cells (Nagel et al., 1982, Clin. Exp. Immunol., 49, 217-224; Schaar et al., 2006, Cancer Chemother. Pharmacol., 57, 789-795) and highlight the ssDNA dynamics upon pharmacological treatment.

In addition, experiments were designed to address what genes had ssDNA openness covarying with the TSS enrichment trend across the five time points. The rationale was that given the correlation between CHI-IEX-seg and active transcription, it would be expected that TPA induced dynamics to be reflected in the nascent transcriptome, which in turn should correlate with C-IEX-seg priming in the gene body. After filtering for genes whose CHEX-seq priming counts (library normalized) had high correlation (|r|≥0.9) with the TSS trend, experiments identified 30 genes that correlated with the TSS trend, and 21 genes that anticorrelated (FIG. 4C). These genes were queried for GO enrichment (FIG. 4D). LRR27 and SHISA6 from ionotropic glutamate receptor binding (GO:0035255) is the only significant function In the correlated genes, while among the anticorrelated genes, several tyrosine kinase functions (GO:0004713, GO:0004715, G0:0030297, GO:0030296) and Ras (GO:0005088) and Ras-like (GO:0017160) functions are enriched (FIG. 4D, bottom). These results revealed the cell signaling and proliferation related pathways show concerted regulation of TSS ssDNA chromatin dynamics. This demonstrates CHEX-seq's capacity to capture immediate and time-delayed changes in the single-stranded chromatin landscape upon chemical perturbation.

### CHEX-Seq Applied to Spatially Identified Cells in Mouse Brain Tissue Sections

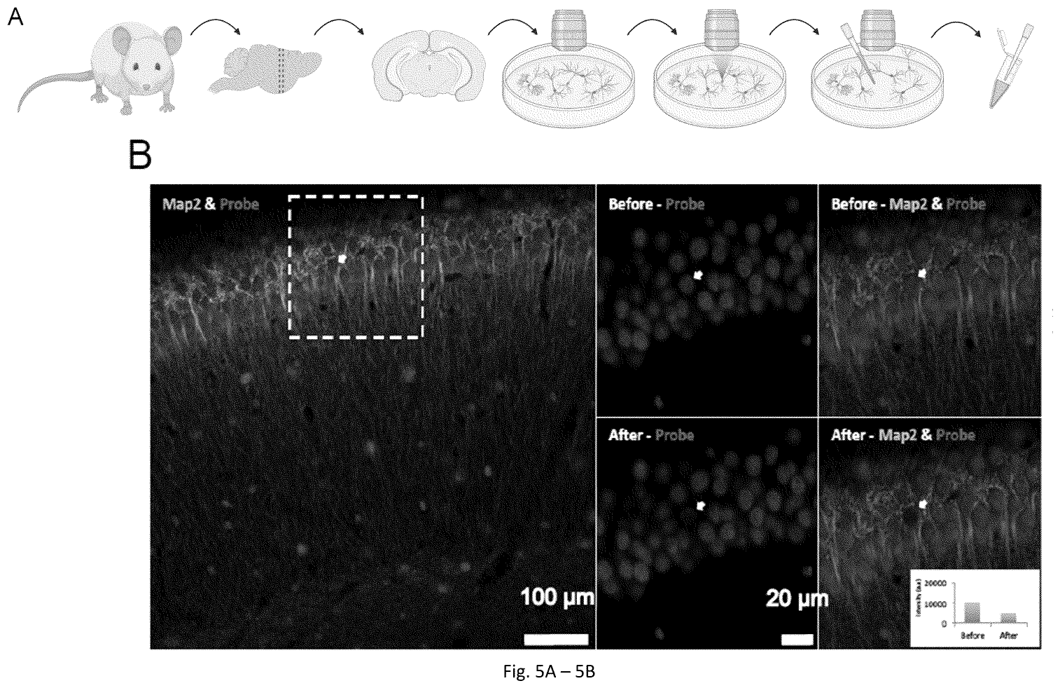

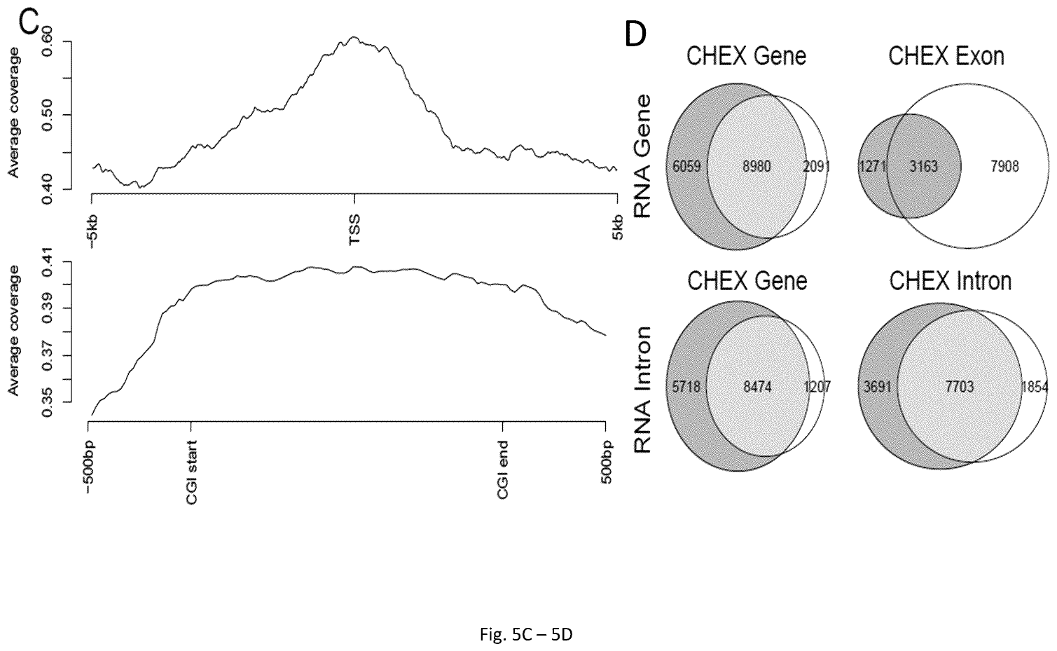

Experiments investigated open-chromatin ssDNA landscape in individual neurons from in situ tissue sections of the adult mouse brain, where neurons were resident in their natural microenvironment (FIG. 5A). CHEX-seq probes were annealed to fixed adult mouse brain coronal tissue sections (100 μm) and detected by immunofluorescence using a fluorescently-labeled antibody targeting the neuronal microtubule associated protein 2 (MAP2) (Shafit-Zagardo et al., 1998, Mol. Neurobiol. 16, 149-162). FIG. 5B shows the CA1 region of the hippocampus stained with MAP2 immunofluorescence (green) and CHEX-seq probes (red). The CHEX-seq probes were then photoactivated in an individual nucleus (arrow in boxed area of FIG. 5B) with the activation confirmed by the loss of fluorescent signal (FIG. 5B, inset).

Experiments first examined read coverage and confirmed the TSS peak when aggregating individual cells from multiple mouse neuron tissue sections (FIG. 5C, top). CpG islands (CGIs) also showed distinct elevation, especially in the area preceding the CGI start (FIG. 5C, bottom). Mammalian CGIs have been found to co-localize with promoters and replication origins (Antequera et al., 2018, Methods Mol. Biol. Clifton NJ. 1766, 3-13; Delgado et al., 1998, EMBO J., 17, 2426-2435; Cayrou et al., 2011, Genome Res., 21, 1438-1449), and these agree with ssDNA's role in transcription, replication, and putatively, DNA methylome maintenance as found in K562. CHEX-seq was repeated in mouse interneuron tissue sections and identified the same TSS peak (FIG. 15C) and the CGI preference (FIG. 15D). Additionally, experiments showed in general higher coverage in the gene-body and a decay from the TSS to the TES (Transcription End Site) in mouse neuron (FIG. 15A) and interneuron (FIG. 15E) tissue section samples. A similar decaying pattern was also observed in the 5′ UTR region in these samples (FIG. 15B, F), however, the functional implication of ssDNA in 5′ UTR is unclear.

Apart from the coverage, experiments compared the genes showing ssDNA from mouse neuron tissue sections with the high-expression transcriptome from single neuron RNA-seq. Like K562, experiments showed an overall significant overlap between CHEX-seq and transcriptome: ˜60% of the ssDNA loci are highly expressed, while 81% of the highly expressed genes exhibit single-stranded regions (FIG. 5D, top-left). This asymmetry suggests a larger proportion of the single-stranded chromatin in fixed tissue sections that is not substantially represented in the transcribed mRNA, and they likely correspond to genes poised to transcribe, DNA replication sites or other types of DNA organizational structures. Further breaking down CHEX-seq or RNA-seq reads into exonic or intronic regions, experiments showed an even stronger overlap with CHEX-seq and RNA-seq in introns (FIG. 5D, bottom-right). As discussed for the K562 CHEX-seq data, intronic transcripts are more indicative of the active transcriptional events hence a better overlap in intronic RNA-seq is unsurprising (FIG. 5D, bottom-left). What was surprising was the better overlap in intronic ssDNA loci: ˜68% of them are highly, actively transcribed, which indicates that in neuron tissue sections, single-stranded intronic chromatin has a better association with ongoing transcriptional activity. This may have to do with introns being longer than exons but may also be reflective of exons generally having a higher density of nucleosomes than exons (Schwartz et al., 2009, Nat. Struct. Mol. Biol. 16, 990-995).

Seeing the overall high CHEX-seq/RNA association in mouse neuron tissue sections, it was hypothesized that a gene's ssDNA level would be positively correlated to the intronic expression level of that gene, and negatively correlated to the intronic expression variability of that gene. Using the number of single neurons that had CHEX-seq reads as a proxy for the ssDNA level, experiments confirmed both: monotonically greater expression level and monotonically lesser expression variability (CV) as the number of CHEX-seq positive neurons increased, especially when it did not exceed 18 cells (FIG. 5E,F). The genes that broke the monocity (on the right-most side) included Kif26b, Upk3bl, Ap3b1, Cadm2, Ctnna3, Gpc5, Mansc1, Ptprm, Tiam2, Mdga2, Tacc2 and Fgfr2; many of them are unusually long. For example, Fgfr2 has an isoform (Fgfr2-217) that contains an intron spanning ˜3 Mb. Ctnna3 and Gpc5 have long isoforms (1.6 Mb and 1.4 Mb, respectively) too. It was therefore suspected that the weakened correlation was partly due to these extremely long genes. It is worth noting that, in general experiments did not show good correlation when using CHEX-seq read counts as the direct readout for the ssDNA level. Two reasons might explain this observation. First, it was hypothesized that, for individual cells CHEX-seq might be more likely a binary gauge for ssDNA—it detects ssDNA by the presence or absence of priming, rather than the frequency of priming. Second, “bursting transcription” has been proposed to model the single-cell transcriptome (Suter et al., 2011, Science. 332, 472-474; Kumar et al., 2015, PLOS Comput. Biol., 11, e1004292). A more highly expressed gene could trigger transcriptional bursts more frequently and the single-stranded state be present more often through the extended periods of bursting: thus, the number of priming cells (as an ensemble) would be a more representative statistic of the ssDNA level than the number of probes annealing to the ssDNA in a single cell.

### CHEX-Seq Replicated in Human and Mouse Brain Dispersed Cultures

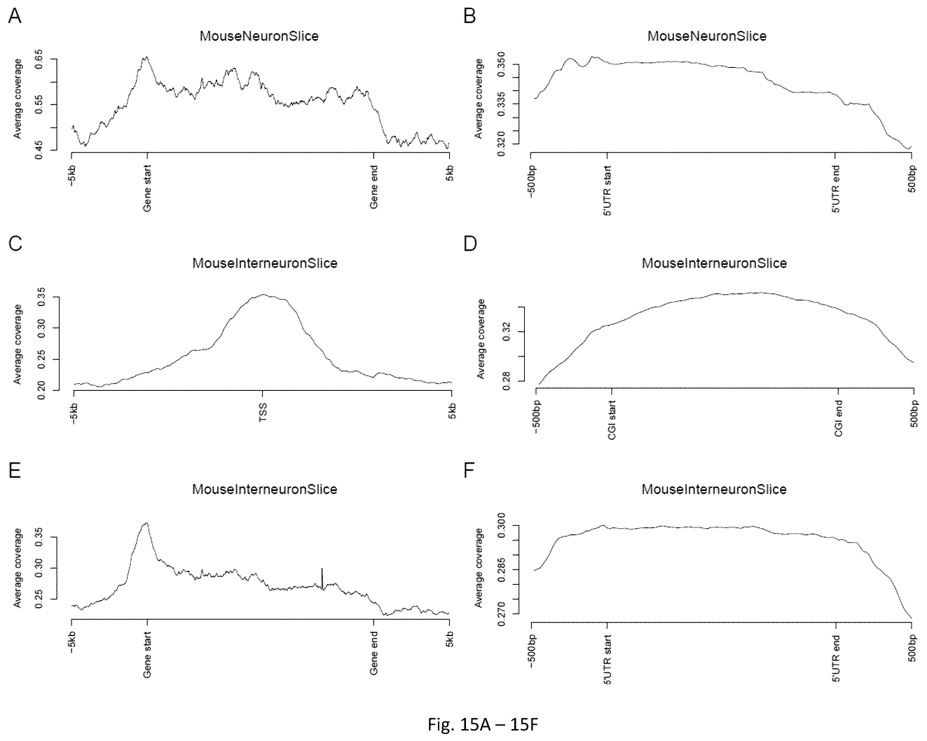

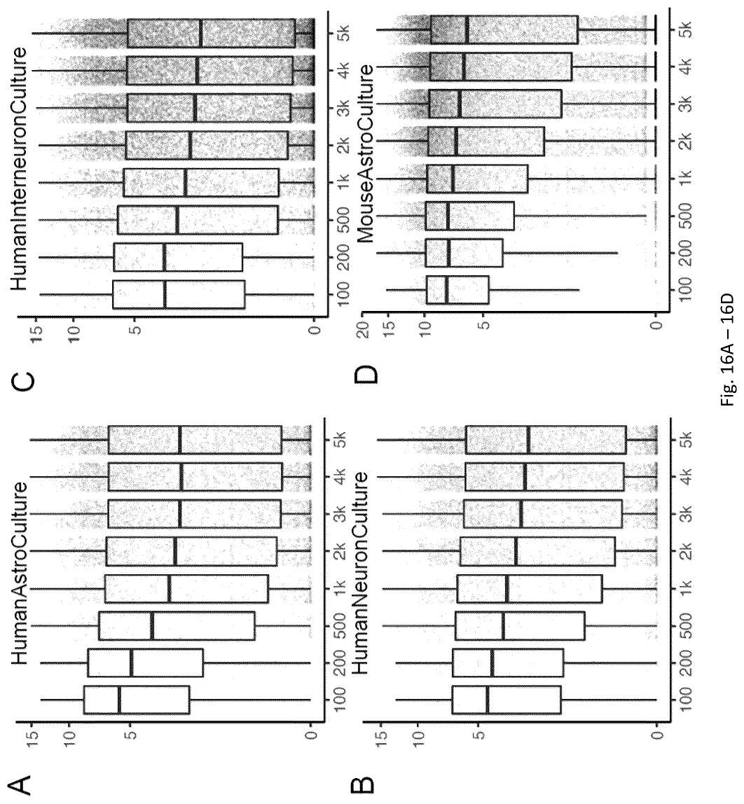

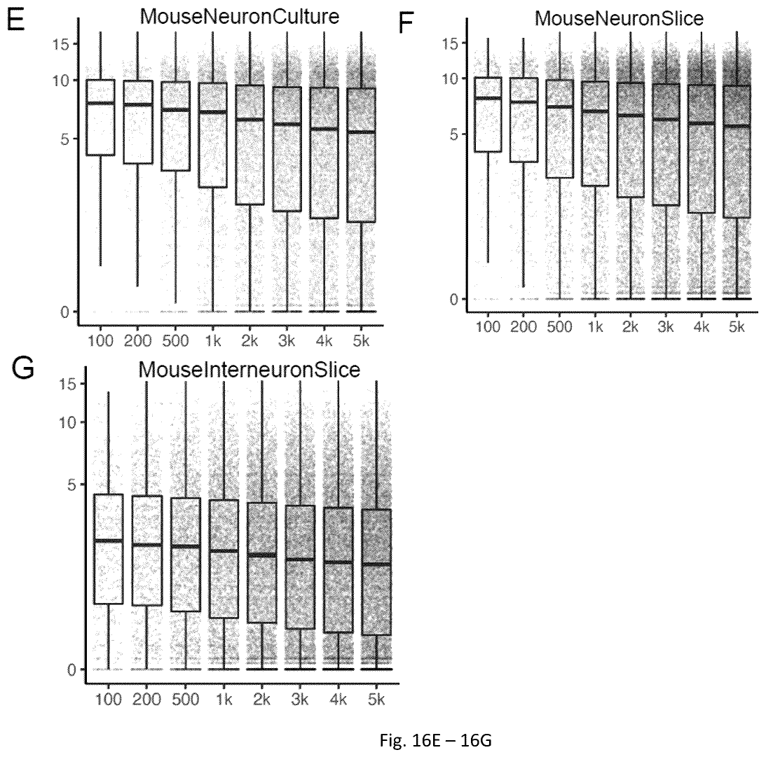

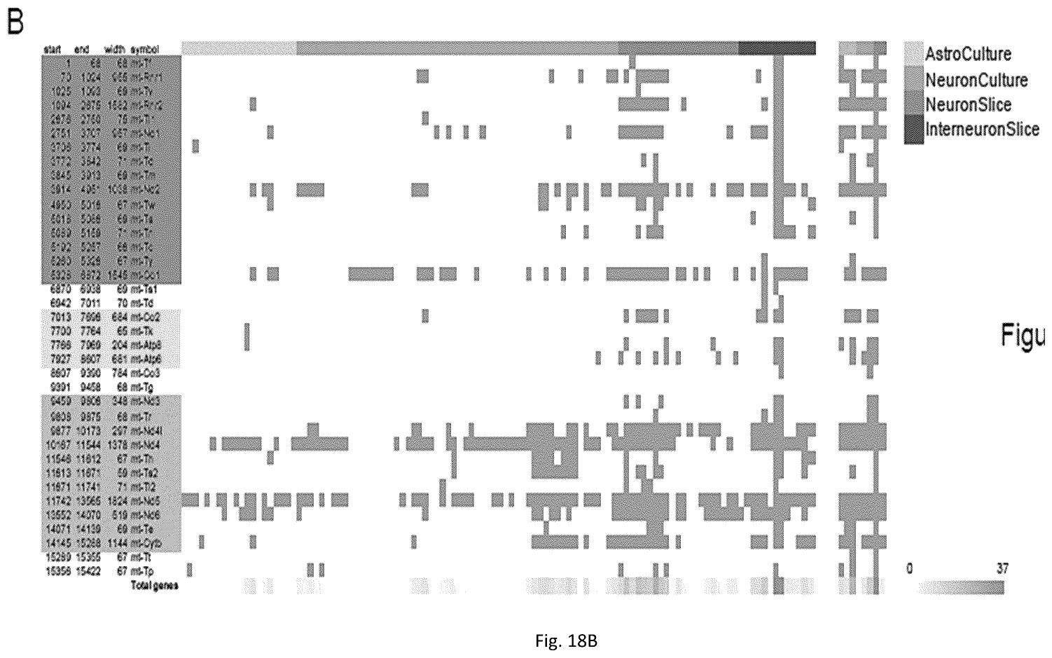

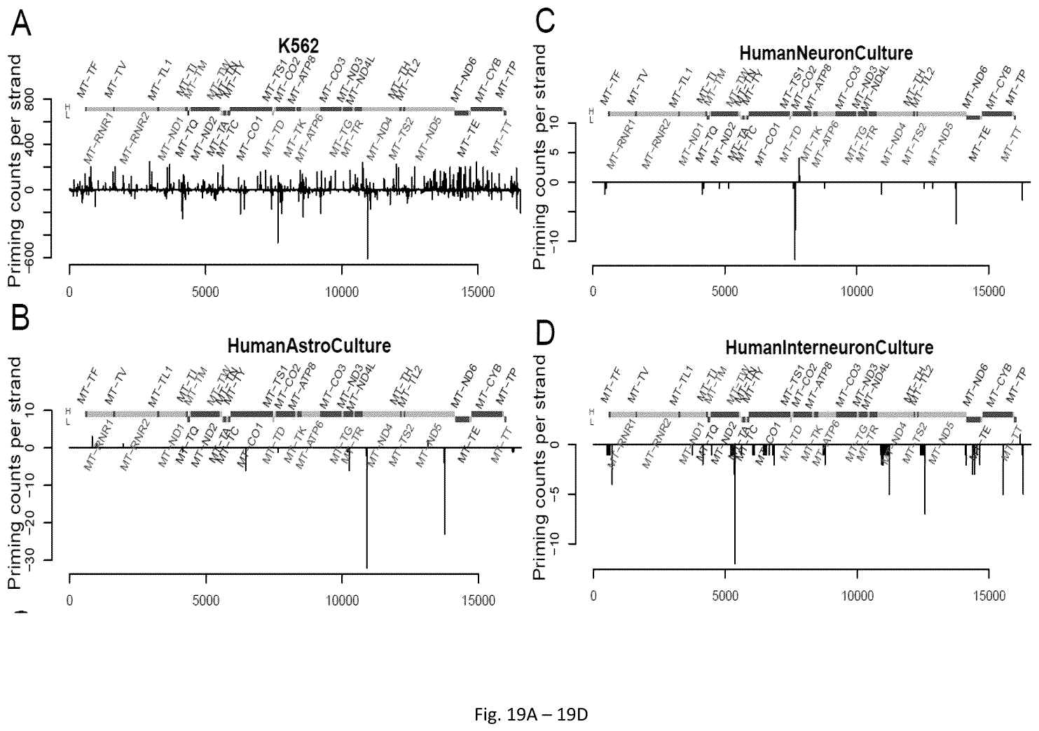

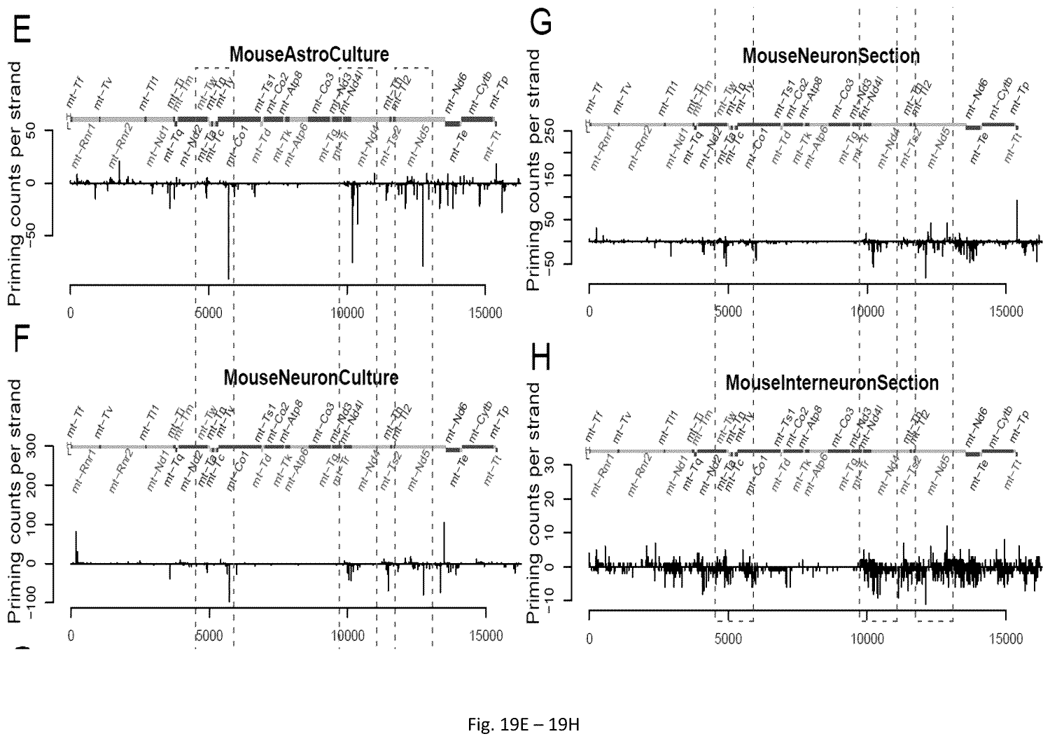

Besides mouse in situ brain sections, CHEX-seq analysis were performed in human and mouse brain primary cultures and were able to recapitulate the relationship between CHEX-seq and transcription, non-B DNA and open-chromatin epigenomes. FIG. 19 showed generally well-correlated, negative relationship between the RNA-seq expression level and the distance of the CHEX-seq priming sites to the TSS (within 5 kb) in human astrocyte (FIG. 16A), neuron (FIG. 16B), and interneuron (FIG. 16C) cultures, and in mouse astrocyte (FIG. 16D) and neuron (FIG. 16E) cultures. The negative correlation was also found for both mouse neuron (FIG. 16F) and interneuron (FIG. 16G) bearing tissue sections. The human and mouse brain samples recapitulated the ENCODE epigenomes enriched in K562. For human astrocyte and neuron cultures, experiments showed consistent and strong enrichment in non-B DNA (direct, short tandem, a-phased), but almost no enrichment in ATAC-seq or FAIRE-seq. This is possibly due to the ENCODE human brain chromatin data from control subjects did not match well with the subjects who presented in the clinic with epilepsy or brain tumors. Nonetheless, ENCODE human DNase-seq showed weak enrichment (˜1.2×) in human astrocyte and neuron CHEX-seq (FIG. 13B). For mouse astrocyte and neuron cultures, as well as neuron and interneuron tissue sections, experiments showed enrichment in non-B DNA (direct, mirror, short tandem, a-phased), ENCODE mouse brain H3K27ac, H3K27me3 and H3K4me3, though to a mild extent (˜1.5×) (FIG. 13C).

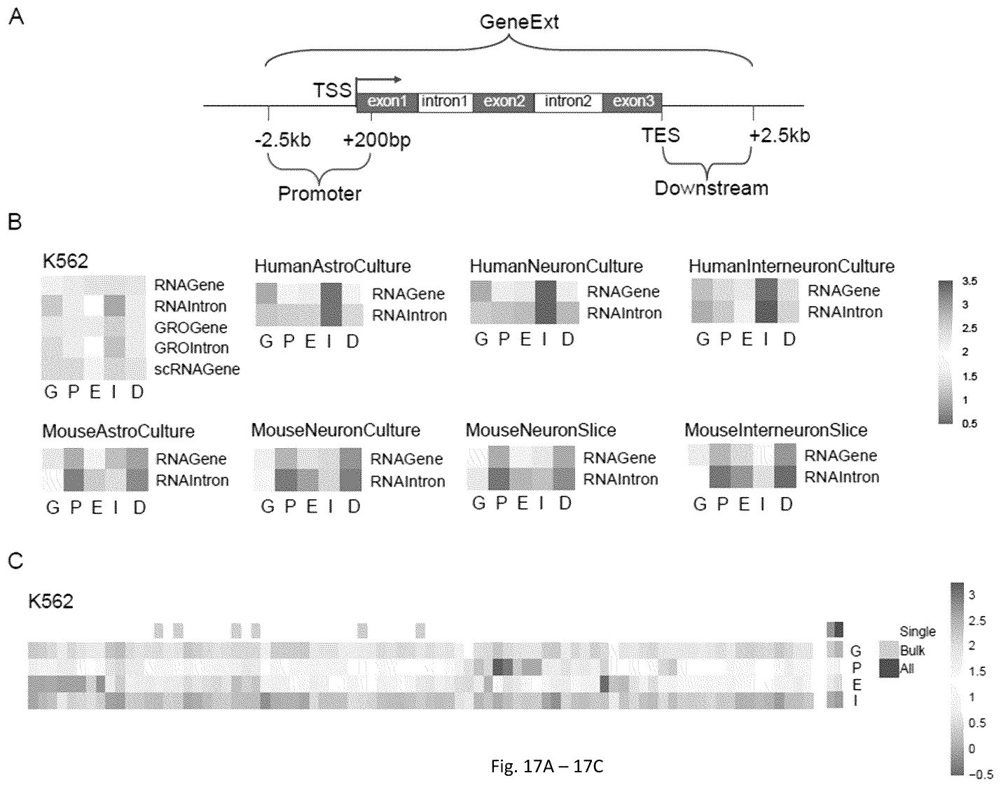

Having observed a tight association between CHEX-seq priming and RNA-seq expression in intronic regions in mouse neuron tissue sections, experiments were designed to determine whether other sub-genic regions also show such associations. To this end, genic units of GeneExt (the gene-body plus 2.5 kb upstream and downstream), Promoter (−2 kb to +200 bp from the TSS), Exon, Intron and Downstream (from the TES to 2.5 kb downstream) were constructed (FIG. 17A). For each (sub-)genic region, the priming counts were pooled from biological replicates and conducted Fisher's exact test for the association between CHEX-seq priming and the highly expressed genes from the RNA-seq of the corresponding cell type. Experiments confirmed that the intronic genic ssDNA correlated best with the intronic gene expression in both K562 and human or mouse neuronal cell samples (FIG. 17B). To further ask whether the CHEX-RNA association exists in single cells, the CHEX-RNA association test was repeated for individual K562 sample. Remarkably, experiments confirmed that genes with intronic ssDNA are significantly associated with highly expressed genes from K562 GRO-seq (FIG. 17C), albeit to a lesser degree as compared to bulk or pooled CHEX-seq samples.

CHEX-Seq Also Detects Mitochondrial ssDNA in Single Cells

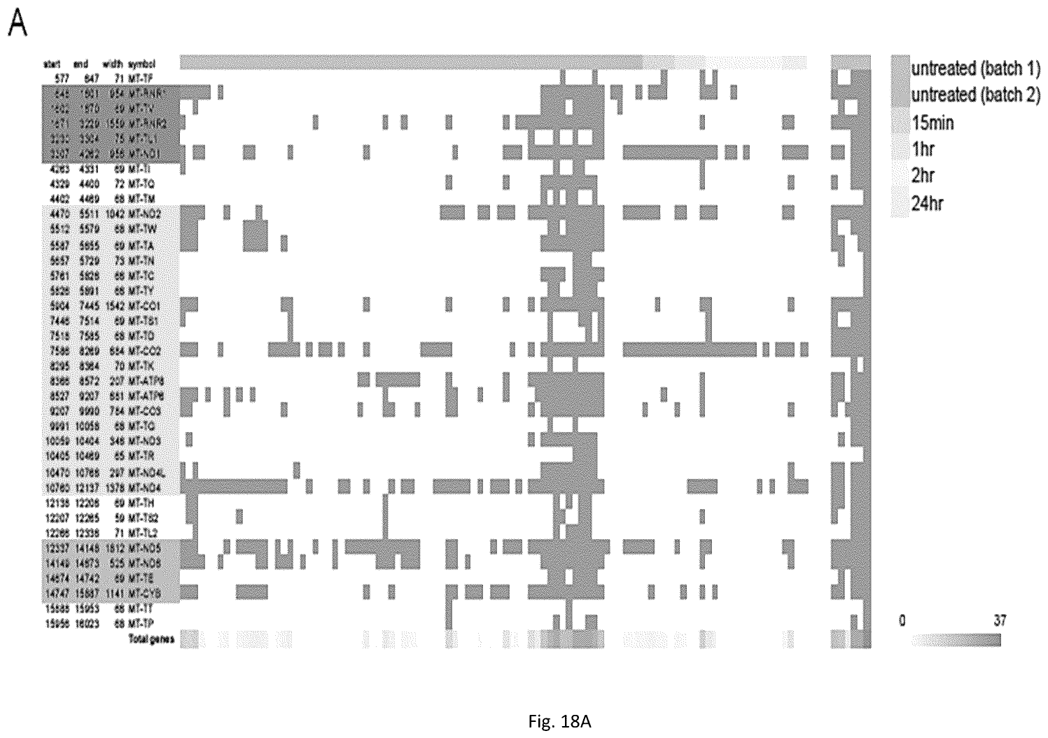

As mitochondria also exist in fixed cells, experiments were designed to address if CHEX-seq could detect single-stranded DNA in the mitochondrial genome. Mitochondrial DNA (mtDNA) has been noted in other open-chromatin assays such as ATAC-seq, but has generally been removed in favor of nuclear DNA analysis (Montefiori et al., 2017, Sci Rep., 7, 2451). Unlike nuclear DNA, mtDNA is not organized into chromatin, but rather has a nucleoid structure (containing ssDNA regions) that is dynamically regulated and transcribed. Since CHEX-seq priming is limited by the interval of single-stranded regions and the mitochondrial genome is only ˜16 kb, many priming events per genic region per mitochondrion were not expected. However, there are usually hundreds of mitochondria per cell and the amount of mitochondrial gene transcription varies between cells, hence CHEX-seq should be able to identify mitochondrial transcribed genes. Indeed, experiments detected ˜7.5 mt-genes with ssDNA detected by CHEX-seq in untreated single K562, and the priming count per gene ranged from 1 to 651 (average 73) (FIG. 18). For bulk K562 samples, the numbers got even higher: on average 24 single-stranded mt-genes and 8570 priming counts per gene; the latter is not surprising because there are as many as 600 cells in a bulk K562 sample.

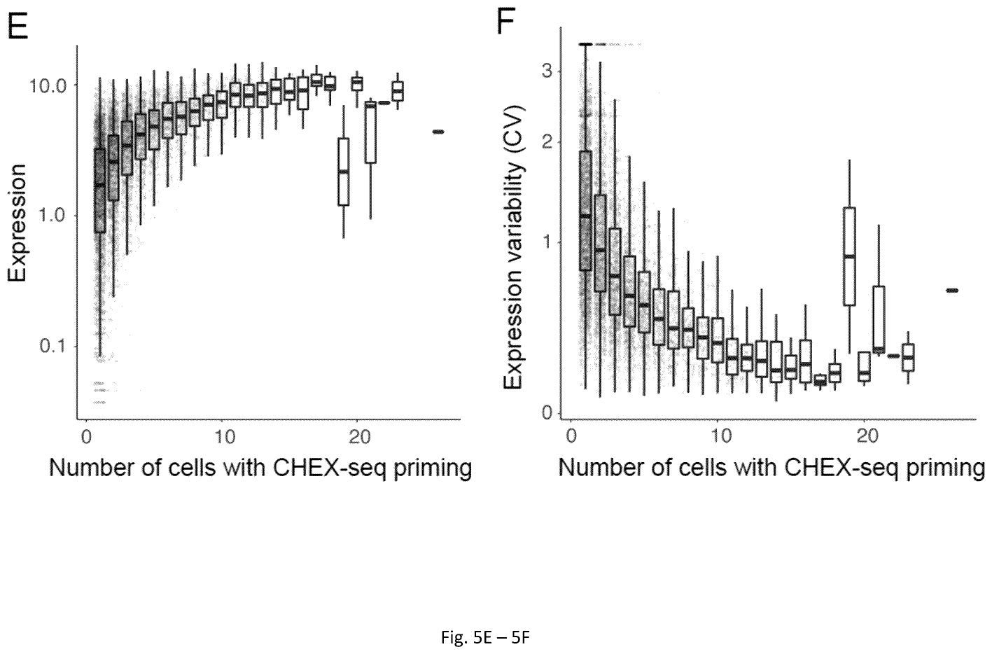

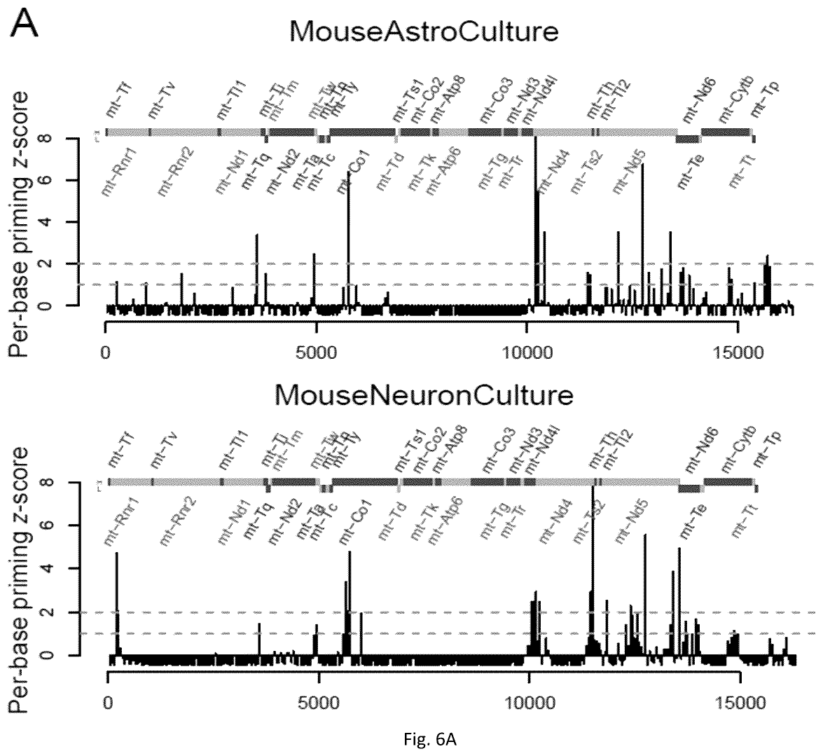

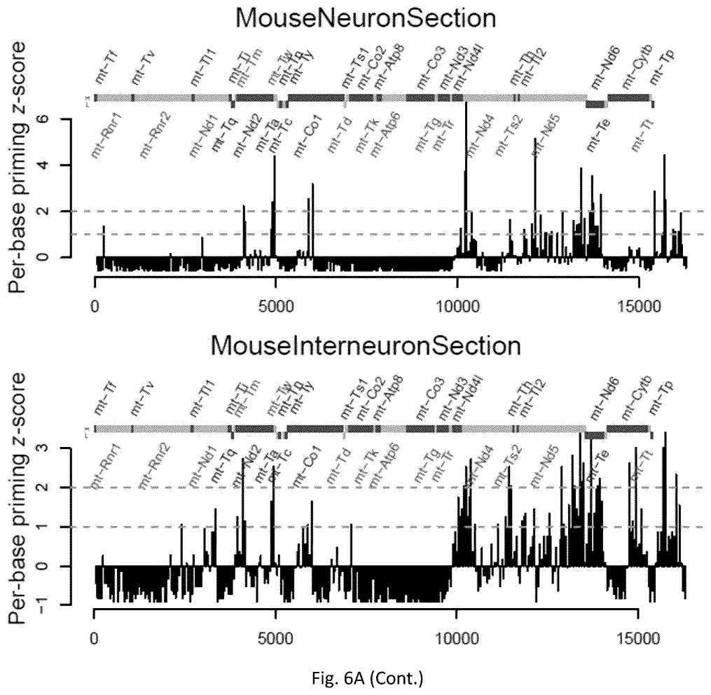

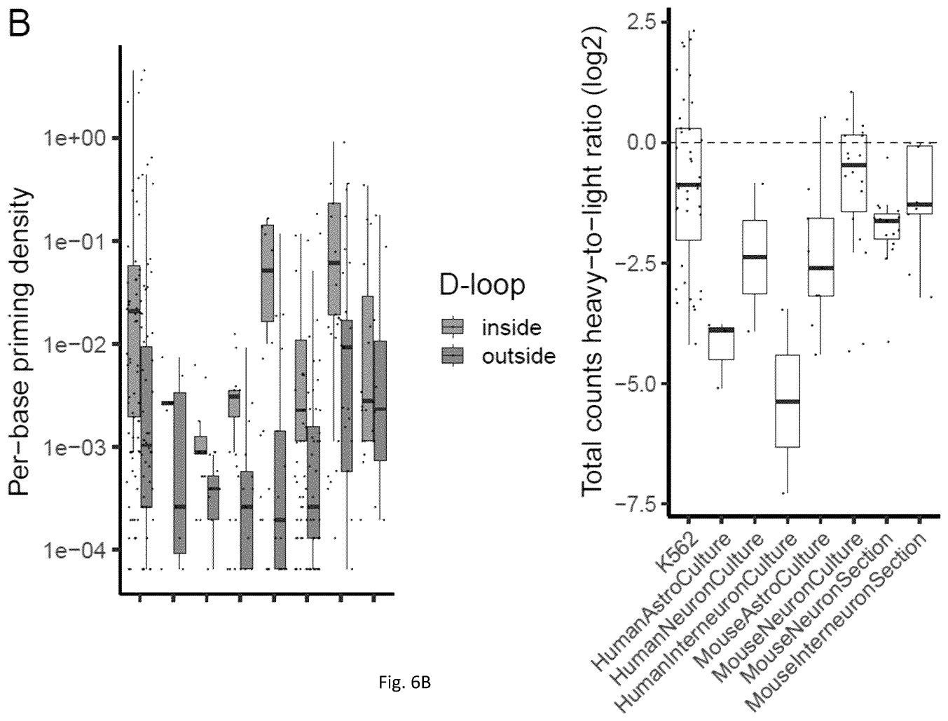

To assess the single-stranded openness along the mt-genome, CHEX-seq mitochondrial priming density were calculated in mouse neuronal cell samples. Experiments showed a nonrandom distribution of priming sites along the circular genome with selected genic regions showing much higher level (≥2SD) of ssDNA, in cells in the in situ tissue section (FIG. 6A): mt-Col, mt-Nd2, mt-Nd41, mt-Nd4, mt-Nd5, mt-Nd6. More interestingly, most of these were shared across the neuronal samples. The astrocytes showed fewer CHEX-seq mitochondrial reads, but several areas were correlated with similar ssDNA regions in the neurons, including mt-Nd2, mt-Col, mt-Nd4, mt-Nd5. This is consistent with prior evidence showing that neurons use more OXPHOS pathways (reflected in more mito transcription) to generate ATP than do astrocytes (Ivanov et al., 2014, J. Cereb. Blood Flow Metab., 34, 397-407; Rose et al., 2020, Front. Neurosci. 14). It is intriguing to note that Map2 immuno-identified cells in the mouse neuron sections and Parvalbumin positive cells in the mouse interneuron tissue section samples showed more mitochondrial ssDNA as compared with primary cultures. Perhaps this is attributable to in situ tissue localized cells being more responsive to their local tissue environment then dispersed cells in primary culture but is also consistent with data showing the PV interneurons have higher metabolic needs (Kann et al., 2014, J. Cereb. Blood Flow Metab., 34, 1270-1282; Kontou et al., 2021, eLife., 10, e65215).

Mammalian mitochondria contain a triplex structure called “D-loop”, which is formed by the displacement of the heavy strand due to the newly synthesized heavy strand annealing to its template (Kasamatsu et al., 1971, Proc. Natl. Acad. Sci. U.S.A, 68, 2252-2257; Marom et al., 2019, iScience., 12, 141-151; Kucej et al., 2008, J Cell Sci., 121, 1861-8). Experiments were designed to ask whether the D-loop has more single-stranded openness for CHEX-seq priming. The per-base priming rate (i.e., counts normalized to the length) was calculated inside and outside the D-loop for human and mouse samples. Indeed, experiments found consistently higher priming rate inside the D-loop than outside it for both species and all cell types (FIG. 6B, top).

As noted, CHEX-seq probes preferentially bind to the strand opposite the template strand that gives rise to the natural, sense-strand mRNAs, resulting in a bias towards the antisense-strand reads (FIG. 3D). Since most of the mt-genes (n=28) are located on the heavy strand (i.e., the light strand being the template strand), it was hypothesized that the heavy strand should be less occupied by the polymerase, which should allow for more CHEX-seq probes binding, thus leading to an excess of CHEX-seq reads mapped to the light strand. To test this model, the heavy-to-light strand ratio of CHEX-seq counts were calculated. Like K562, all human and mouse data showed systematic bias towards the light-strand (FIG. 6B, bottom; FIG. 19), which matched this prediction.

Genomic ssDNA can Act Catalytically to Metalate Porphyrin In Vitro

Apart from passively serving as a genomic template, experiments were designed to test whether endogenous ssDNA could conduct active functions. There is precedent for endogenous single stranded nucleic acids having catalytic activity as some endogenous single-stranded RNAs can act enzymatically and are so-called “ribozymes” (Hammann et al., 2007, Genome Biol. 8, 210; N. K. Tanner, 1999, FEMS Microbiol. Rev., 23, 257-275). Ribozymes are transcribed from genes in the genome but can be engineered in vitro (Breaker et al., 2003, RNA N. Y. N., 9, 949-957) via systematic evolution of ligands by exponential enrichment (SELEX) (Gopinath et al., 2007, Anal. Bioanal. Chem. 387, 171-182). Likewise, in vitro synthesized ssDNAs (“DNAzymes”) are able to catalyze various reactions including cutting and modifying RNA molecules (Baum et al., 2008, Cell. Mol. Life Sci. CMLS. 65, 2156-2174; Cairns et al., 2004, Methods in Molecular Biology, 267-277; Silverman et al., 2005, Nucleic Acids Res. 33, 6151-6163), as well as modification of small molecules such as porphyrin metalation (Yang et al., 2021, Chem. Front, 8, 2183-219). No endogenous DNAzymes have been described as gDNA has generally been thought to exist predominantly in double-stranded B-form that precludes the DNA from acting catalytically. In assessing potential functionality of CHEX-identified ssDNA, these areas for sequence similarity were screened with published DNAzyme sequences. In human and mouse genome respectively, experiments identified ˜300 and ˜900 single-stranded loci with high homology to previously engineered synthetic DNAzymes, including those that had been synthesized to metalate porphyrin.

Porphyrins are present in all mammalian cells (Sachar et al., 2016, J. Pharmacol. Exp. Ther., 356, 267-275). They are critical metabolic precursors to heme and are involved in other biological processes including neuroprotection (Chiabrando et al., 2018, Front. Neurosci. 12; Lee et al., 2015, Angew. Chem. Int. Ed. 54, 11291-11291; Yu et al., 2016, Sci. Rep., 6, 24200; Daly et al., 2015, Chem. Commun. Camb. Engl., 51, 1066-1069). Some metalated porphyrins are known to bind to DNA (Tanner et al., 1999, FEMS Microbiol. Rev., 23, 257-275; Breaker et al., 2003, RNA N. Y. N., 9, 949-957; Baum et al., 2008, Cell. Mol. Life Sci. CMLS. 65, 2156-2174). The binding to DNA can occur through intercalation of the porphrin macrocycle between the bases or external to the nucleotide rings, dependent upon the type of porphyrin and metal ion. Porphyrins have also been shown to interact with DNA often through intercalation in G4-DNA quadruplexes (Cavallari et al., 2009, J. Phys. Chem. B., 113, 13152-13160) including direct interaction with Bcl-2 promoter G-Quadruplex (Le et al., 2013, PLOS ONE, 8, e72462). Further, metalloporphyrins appear to bind to ssDNA (Sehlstedt et al., 1994, Biochemistry, 33, 417-426.

Of the several ssDNA regions predicted to contain gDNAzyme (genomic DNAzyme) sequences, a subset of those that correspond to DNAzymes that metalate porphyrin were selected to screen for enzymatic activity. Several prospective gDNAzymes were screened for enzymatic activity with some showing no activity. Three of the genomic regions that showed activity include: two from human RPL7AP61 (chr13:97852922-97852939) and TATDN2P1 (chrX:44290573-44290591) and one from mouse Bmpr1 (chr14:34429016-34429034) (FIG. 7).

As synthetic DNAzymes range in size between 20-50 nucleotides, 49 nt sequences were synthesized from the CHEX-identified ssDNA regions. Previously, metalation of porphyrin with Zinc (Zn⁺⁺) has been observed with molecularly evolved synthetic DNA sequences and, in some cases, it has been shown that it can be enhanced by adding lead (Pb⁺⁺) as a cofactor (Peng et al., 2019, Anal. Chem., 91, 11403-11408). In these studies, genomic DNAzyme (gDNAzyme) candidates were incubated with mesoporphyrin IX (mPIX), Zn²⁺ and Pb2+ and observed a time-dependent increase in the insertion of Zn²⁺ into mPIX. The kinetic curves plateaued at 8-12 hours (FIG. 7A) and revealed statistically significant increases in the rate of metalation (FIG. 7B). Reactions without the Pb²⁺ cofactor showed little activity (FIG. 7A). As a negative control, isolated mouse liver gDNA was also screened for innate catalytic activity. Both native and denatured gDNA showed a slow but steady non-saturated increase in porphyrin metalation, whose rate was higher than that of reactions without Pb²⁺ cofactor (FIG. 7A, C). The fraction of the total gDNA sequence that contains predicted porphyrin metalation gDNAzyme sequences is small, suggesting that this limits the attainable molar concentration of a gDNAzyme in isolated gDNA for testing in the in vitro assay.

As the endogenous ssDNA regions have structures that are constrained and stabilized by inter- and intra-strand base pairing as well ssDNA binding proteins, the effect of sequence constraints upon porphyrin metalation activity were tested. To assess this, a 90 nt gDNAzyme oligonucleotide (“long”) was synthesized to which a complementary clamping oligonucleotide which anneals to internal regions of the long oligonucleotide, forcing a short (45 nt) single-stranded loop containing the gDNAzyme sequence to form (“short loop”) (Table 2). A second clamping oligonucleotide that anneals to the ends of the long oligonucleotide was used to force a long single-stranded 66 nt loop to form (“long loop”). The short (49 nt) single-stranded oligonucleotide (“short”) that had showed porphyrin metalation activity was compared to other TATDN2P1 DNA structural variants. The long construct showed the least amount of metalation activity, whereas the short loop showed increased activity and the long loop showed activity that is comparable to the original short sequence (FIG. 7D).

To determine the endogenous genomic localization of the ssDNA that contain putative gDNAzymes, a sensitive two-oligonucleotide Forster resonance energy transfer (FRET) fluorescence in situ hybridization (FRET-FISH) procedure was performed (FIG. 7E). Given the short size of the gDNAzyme to be detected, this FISH procedure is different from that used to assess longer stretches of CHEX-identified ssDNA (FIG. 14). For FRET to occur the donor chromophore on the 3′-end of one oligonucleotide must be close to the acceptor on the 5′-end of an adjacent oligonucleotide with the oligonucleotides separated by 5 bases. Detection of the FRET signal was aided by use of a high-resolution analysis procedure that quantitates user-specified subfractions of the excitation and emission spectra for the FRET pairs (Table 2). This approach reduces artifacts caused by autofluorescence and other background signals. The in situ DNA localization of the porphyrin metalating gDNAzyme in mouse Bmpr1 was assessed with the FRET-FISH probe-annealed region (red dots) is shown in three cells localized to the periphery of the nucleus in areas occupied mostly by heterochromatin (Penagos-Puig et al., 2020, Front. Cell Dev. Biol., 8, 579137) (FIG. 7E). This is consistent with the fact that many of the CHEX-seq reads originated from the repetitive genome. As expected, not all cells showed signal likely due to dynamics of double-stranded/single-stranded DNA transitions (FIG. 4A).

CHEX-Seg Detects Single-Cell Genomic ssDNA

Light-assisted spatially activated CHEX-seq queries single-stranded open-chromatin DNA regions in single cells. As an interrogator of ssDNA in single cells, CHEX-seq assesses not only nuclear DNA but also the single-stranded open-chromatin status of the mitochondrial genome. As a measure of chromatin openness and surrogate for gene transcriptional activity, the ability to assess the transcriptional potential of fixed cells provides a window into the plasticity that underlies a cell's ability to respond to local cues. This is particularly important in understanding the plasticity of neuronal systems where neurons must respond to environmental influences generated through the activity of synaptically interconnected distal neurons as well as local cellular interactions. As a component of cellular phenotype, coupling chromatin status with immunocytochemical categorization will help link a cell's plasticity with its function. Light activation of chromatin state analysis enables the connection to function to be more concrete using fluorescent biomarkers of physiological function that can be imaged at the same time as when chromatin analysis is initiated.

The strandedness of annealing of CHEX-seq primers to ssDNA, apparent in the upstream of transcribed genes, suggests a scaffolding of proteins on one strand of the DNA (blocking CHEX-seq probe binding). This finding is intriguing given the dynamic back and forth between genes' double- and single-stranded status upon chemical perturbation, which emphasizes the role of chromatin 3D structure in transcriptional regulation. It is tempting to hypothesize that the balance between the single and double-strand states of chromatin and the rate of interconversion is important to the degree of plasticity or vulnerability that any cell can exhibit.

The surprising discovery that regions of the endogenous genome have sequence similarity with manufactured DNAzymes and that these sequences exhibit in vitro catalytic activity suggests a novel means for cells to modulate selected biological processes. It is important to note that the in vivo demonstration of DNA catalytic activity remains to be shown. In particular, for the example of gDNAzyme catalyzed porphyrin metalation, the requirement of Pb++ as a cofactor for catalysis of porphyrin metalation suggests that in vivo porphyrin metalation through this mechanism is unlikely as high concentrations of Pb++ are cytotoxic. These data do however show that a subset of CHEX-identified ssDNA loci have the capacity to act catalytically. Any such in vivo activity would require the ssDNA to be in a structure where the active site of the catalytic ssDNA sequence is free to take on a conformation that facilitates its enzymatic activity. The short single-strand porphyrin gDNAzyme shown in FIG. 7D seemingly assumes such a catalytic structure while the long single-stranded oligonucleotide likely exists as a disordered structure that hinders the sequence from acting enzymatically. This hypothesis is supported as when the long oligonucleotide sequence is constrained to produce a ssDNA loop (FIG. 7D) the long ssDNA can act catalytically likely due to assuming a favorable conformation. The longer loop duplex is presumed to have more degrees of freedom to conform to the porphyrin substrate and hence exhibits greater enzymatic activity than the short, more constrained loop duplex. As the ssDNA landscape dynamically varies between single cells (FIG. 4A and FIG. 7E), it is difficult to predict what regions of gDNA may be available to act catalytically at any time. We note that in addition to porphyrin metalation several other synthetic DNAzymes with varying catalytic functions (including RNA cleavage and DNA ligation) have sequence similarity to CHEX-identified single-stranded regions in genomic as well as mitochondrial DNA. It is intriguing to speculate that genomic and mitochondrial gDNAzymes catalyze enzymatic reactions provide locally required compounds.

The association of in situ single-stranded non-B-form DNA in transcribed genes, especially their intronic regions, with gene transcription as well as its presence in intergenic areas suggests an important regulatory role for the dynamic interconversion and ratio of single- to double-stranded DNA in chromatin dynamics. As such, the ability to perform single-cell, spatial open-chromatin, non-B-form DNA analysis on immuno-identified single neuronal cells promises to provide high resolution spatial genomic landscape analysis of dynamic circuit function and disease-associated dysfunction in functionally relevant cells.

### Example 2: High-Throughput Identification and Characterization of Endogenous ssDNAs that Exhibit Catalytic Potential to Modify RNA and DNA

Homology searches to identify endogenous gDNAzymes are limited by the small repertoire of synthetic DNAzymes that have been developed and evolved to perform a restricted set of predetermined functions. Herein, novel functional screens (DEAS methodology) are employed to de novo identify the cellular repertoire of endogenous gDNAzymes from amongst CHEX-seq identified ssDNA regions. Screening by function overcomes the limitations imposed studying only those candidate gDNAzymes with homology to the limited number of synthetic DNAzymes that have been in vitro evolved to perform specified functions.

Specialized High-Throughput DEAS Screen for gDNAzymes that Cleave DNA

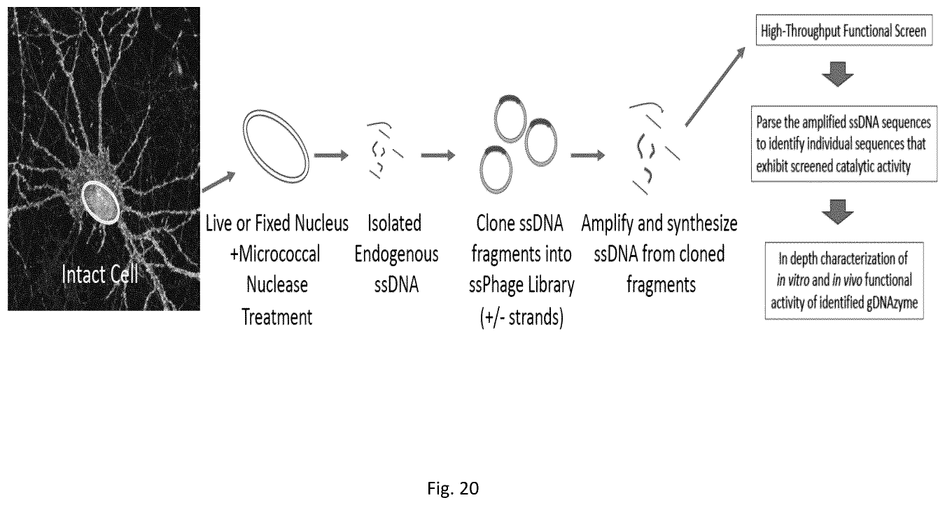

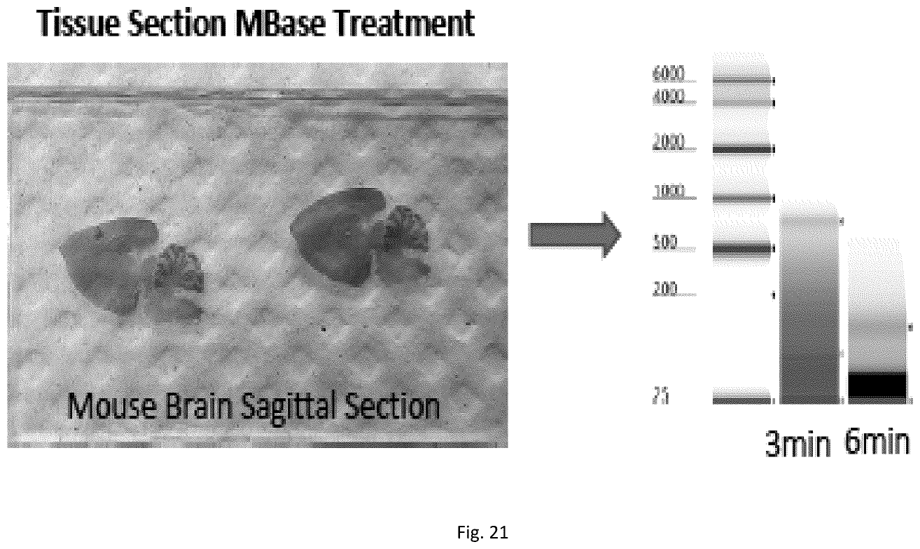

These “first in class” predicted gDNAzymes were identified using homology-based approaches to screen databases of known synthetic DNAzymes. This approach is inherently limited as synthetic DNAzymes have been evolved to perform specific user-defined functions with selective substrates such as small molecules like porphyrin or as modifiers of specific RNA/DNAs including cleavage of particular RNAs. As such the manmade compendium of DNAzyme will only identify a small subset of endogenous gDNAzymes that is anticipated to be quite large given the large number of CHEX-detected ssDNA regions throughout the genome. Their catalytic activities may be quite varied and is difficult to predict. A general high-throughput DNA Enzymatic Activity Screen (de novo gDNAzyme sequences; DEAS) is described that utilizes genome-wide endogenous ssDNAs as catalysts in various easily quantified reactions. This general approach for screening ss gDNA for catalytic function utilizes either—1) isolated intact nuclei from neurons (FIG. 20) to preserve endogenous ssDNA complexity (the nuclei are isolated from neurons treated with a variety of pharmacological agents) and to modulate the amount of single-strandedness and/or 2) ssDNA isolated from fixed dispersed primary cell cultures or from fixed tissue sections that were subjected to these same pharmacological agents. To isolate the ssDNA from paraffin embedded fixed mouse brain tissue the sections are placed in a glass slide holder filled with xylene and left for 3 min, this is repeated with fresh xylene. The sections are then dipped into a 1:1 mixture of xylene and 100% EtOH for 3 min of incubation. The tissue sections are hydrated gradually through graded EtOHs. 100 μl of Mung Bean Nuclease reaction mixture was added to the tissue sections for 3 min at 370 (FIG. 21), stopped with EGTA, purified, and run on a bioanalyzer to quantify the amount of isolated ssDNA (FIG. 21).

Different gDNAzyme sequences are available because of pharmacologically stimulated chromatin changes and associated changes in ssDNA gDNAzyme availability depending upon the manipulation. The pharmacological manipulations used for primary cell culture are 1) treatment with 90 mM KCl for 3 min, wash for 10 min and repeated 4 times (I.-F. Wang et al., 2008, Journal of Neurochemistry 105, 797-806), and 2) treatment with 100 mM DHPG for 1 hour to stimulate metabotropic glutamate receptor (Huber et al., 2002, Proceedings of the National Academy of Sciences 99, 7746-7750; Kolber et al., 2010, J. Neurosci. 30, 8203-8213). Upon isolation, ssDNA endonuclease (e.g., Mung Bean Nuclease, Endonuclease IV, etc.) is diffused into the nuclei where it cleaves endogenous ssDNA. At various times the reaction is stopped and the liberated ssDNA isolated. The purified ssDNA is amplified by cloning into single-strand phage and phagemid vectors (e.g., M13) enabling “+ and −” strand ssDNA to be created (Szekely et al., 2016, FEMS Microbiology Letters 363, fnw027; Zagursky et al., 1984, Gene 27, 183-191). After propagation, the single-strand phage DNA is screened directly for the assayed catalytic activity.

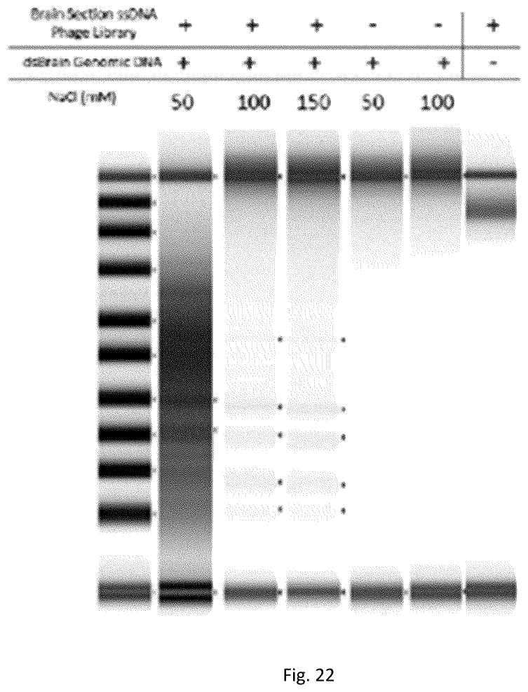

The use of DEAS to screen for endogenous gDNAzymes that cut genomic DNA is shown in FIG. 22. In these experiments brain tissue ssDNA that has been cloned into M13 phage was incubated with mouse double-stranded genomic DNA for 2 hour under varying reaction conditions. The phage cloned ssDNA and genomic DNA controls are shown in FIG. 22, lanes 5-7 of the tape station image (FIG. 22, lanes 5-7) while the enzymatic products (bands and smear below intact genomic DNA and intact phage) are shown in lanes 2-4 after incubation with differing concentrations of NaCl. 50 mM NaCl was better for cleavage than the higher concentrations. These gDNAzyme cleavage products are sequenced to identify the genic regions that were cut by the gDNAzyme library. These genic regions are used as bait to fish out the DNAzyme that have activity on the specific DNA sequence (e.g., nuclease activity).

This system easily enables user-defined changes in size and structural constraints on the cloned ssDNAs, facilitating their screening for gDNAzyme activity. For instance, since the size of the functional catalytic unit is unknown, the ssDNA Phage libraries are treated with ssDNA endonuclease (to shorten the DNA) and the smaller fragments will also be screened for activity. For pools of phage showing catalytic activity, specific catalytic sequences are identified 1) through iterative sub-partitioning of the phage libraries using the activity assay as a screen and 2) sequencing of the substrate DNA product with the goal of finding those subsets of reads where the 3′-end of one read is near the 5′-end of a second read. In this second strategy, identification of those “paired” reads will suggest that the larger encompassing genomic DNA was cleaved between these fragments and the region between the reads can be used as a specific sequence-substrate to “bootstrap” the isolation of the catalytic gDNAzyme.

Specialized High-Throughput Screen for gDNAzymes that Cleave RNA

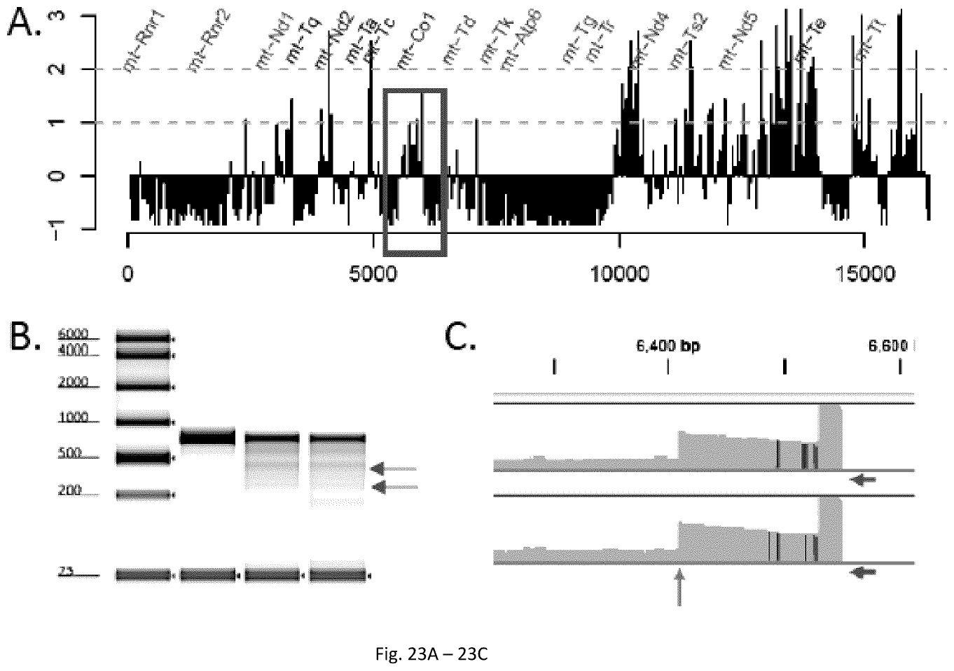

Among the neuronal CHEX-seq predicted gDNAzymes is a mitochondrial DNA region present in the Cox1 gene which has homology with a synthetic DNAzyme that cleaves RNA (FIG. 23A). The predicted mtDNAzyme sequence was synthesized and tested in vitro to determine whether it would cut Cox1 RNA at the predicted cleavage site. Indeed, FIG. 23B shows that an in vitro synthesized 1200 nt portion of Cox1 mRNA was cut giving rise to the predicted size fragments which were sequenced to confirm cutting site. To assess this in vivo, the same primer extension assay was used on RNA isolated from mouse brain cortex. Of the primer extended products ˜80% of the detected endogenous Cox1 were full-length with the remaining 20% exhibiting various deletions and/or 5′-ends. ˜6% of these RNA isoforms have a 5′-end coincident with the predicted cut site for the Cox1-directed mtDNAzyme. These in vivo data show the endogenous catalytic activity of mito ssDNA.

The DEAS assay is customized to identify endogenous gDNAzymes that exhibit RNA cleavage activity. In this multi-RNA analysis screen the cloned cellular ss genomic DNA phage libraries are incubated with isolated size-selected cellular RNA, with a length of >1 kb. After incubation for varying times, under varying reaction conditions, the reaction mixture is screened for cleaved RNA fragments that are <1 kb in length. With appropriate controls implemented, fragments below 1 kb can only result from catalytic activity of the in vivo CHEX-defined phage cloned ssDNA. RNA fragments that are <1 kb in length are isolated and sequenced to identify the RNAs that are cleaved by the gDNAzymes. Full-length mRNA for the identified RNA is used as a “fishhook” to bootstrap isolate the gDNAzyme that performs the cleavage. With this in mind, to gain a wider range of functional gDNAzymes where bands might be faint due to there being a small amount of substrate RNA in the original cellular pool of RNAs or if the in vitro activity is not optimal, then the whole range of RNAs below 1 kb can be cloned.

DEAS can be modified to screen for gDNAzymes with other activities such as phosphorylation of DNA or RNA, DNA or RNA ligation, etc.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising an isolated catalytic single stranded DNA molecule (DNAzyme) or a modulator of a catalytic single stranded DNA molecule (DNAzyme).

2. The composition of claim 1, wherein the modulator comprises an inhibitor of the DNAzyme. wherein the inhibitor disrupts or destabilizes the conformation of the DNAzyme.

3. The composition of claim 1, wherein the modulator comprises an activator of the DNAzyme, wherein the activator generates or stabilizes a specific structure within the ssDNA region to increase the activity of the DNAzyme.

4. The composition of claim 3, wherein the activator comprises an oligonucleotide comprising at least one region that binds to an internal ssDNA region of the DNAzyme.

5. The composition of claim 4, wherein the activator comprises an oligonucleotide comprising at least one region that binds to an internal ssDNA region of the DNAzyme, wherein binding of the oligonucleotide generates or stabilizes a specific structure within the ssDNA region.

6. The composition of claim 5, wherein the activator comprises a clamping oligonucleotide selected from the group consisting of SEQ ID NO:29 and SEQ ID NO:30.

7. A method modulating the level or activity of a DNAzyme, the method comprising contacting the DNAzyme with a composition of claim 1.

8. The method of claim 7, wherein the DNAzyme is selected from the group consisting of SEQ ID NO:28, SEQ ID NO:27 and SEQ ID NO:32.

9. The composition of claim 1 comprising an isolated nucleic acid molecule comprising a porphyrin metalation DNAzyme, wherein the DNAzyme is selected from the group consisting of SEQ ID NO:28, SEQ ID NO:27 and SEQ ID NO:32.

10. A method of modulating porphyrin metalation in a sample, the method comprising contacting the sample with a composition of claim 9.

11. The method of claim 10, wherein the method comprises contacting the sample with a complex comprising
a) a DNAzyme selected from the group consisting of SEQ ID NO:28 and SEQ ID NO:27; and
b) a clamping oligonucleotide selected from the group consisting of SEQ ID NO:29 and SEQ ID NO:30.

12. A method of treating a disease or disorder in a subject in need thereof, the method comprising administering a modulator of claim 1 to modulate the activity of a DNAzyme in the subject, wherein the subject has a disease or disorder that would benefit from modulation of the DNAzyme.

13. The method of claim 12, wherein the disease or disorder is selected from cancer, a neurodegenerative disease, a mitochondrial disease, a disease associated with a DNA repair defect, a disease associated with telomere shortening, a bacterial disease and a viral disease.

14. A method of identifying a DNAzyme, the method comprising
a) performing a CHEX-seq (CHromatin EXposed) assay to identify single-stranded open chromatin regions in a genomic or mitochondrial DNA sample,
b) identifying at least one catalytic core sequence of a DNAzyme having a desired activity,
c) aligning the catalytic core sequence of the DNAzyme having the desired activity with the single-stranded open chromatin regions in a genomic or mitochondrial DNA sample,
d) identifying homologous ssDNA loci in the sample as potential DNAzymes, and
e) confirming the DNAzyme activity of the homologous ssDNA loci.

15. The method of claim 14, wherein the method further comprises a step of synthesizing a modulator of the DNAzyme.

16. A method of identifying a catalytic nucleic acid molecule, the method comprising performing a de novo gDNAzyme sequences (DEAS) assay comprising the steps of:
a) isolating endogenous single stranded DNA from at least one genonic or mitochondrial DNA sample;
b) cloning at least a portion of the isolated ssDNA into a single-strand phage to generate a library of phage-cloned ssDNA;
c) contacting the phage-cloned ssDNA library with at least one test nucleic acid molecule under conditions that allow for the generation of modified nucleic acid products by a catalytic nucleic acid molecule;
d) collecting and sequencing the modified nucleic acid products to identify the target sequence of a catalytic nucleic acid molecule, and
e) contacting at least a portion of the sample with a bait nucleic acid molecule comprising the target sequence of the catalytic nucleic acid molecule to capture the catalytic nucleic acid molecule.

17. The method of claim 16, wherein the method further comprises a step of sequencing the captured DNAzyme.

18. The method of claim 16, wherein the method further comprises a step of contacting the ssDNA phage library with ssDNA endonuclease prior to step c).

19. The method of claim 24, wherein at least one test nucleic acid molecule of step c) is a genomic DNA molecule or RNA molecule.

20. A catalytic DNA molecule isolated according to the method of claim 16.

21. The catalytic DNA molecule of claim 20, wherein the catalytic nucleic acid molecule is a gDNAzyme or an mtDNAzyme.

22. The catalytic DNA molecule of claim 20, wherein the catalytic nucleic acid molecule has DNA cleavage activity, RNA cleavage activity, DNA ligation activity, RNA ligation activity, DNA phosphorylation activity or RNA phosphorylation activity.


SEQUENCE LISTING (37 sequences)

SEQ ID NO: 1 - DNA, 34 bp, synthetic construct
  source 1..34: mol_type = other DNA
gcgccattgaccaggattttcccccccccccccc

SEQ ID NO: 2 - DNA, 21 bp, synthetic construct
  source 1..21: mol_type = other DNA
gcgccattgaccaggattttc

SEQ ID NO: 3 - DNA, 20 bp, synthetic construct
  source 1..20: mol_type = other DNA
tagggagacgcgtgatcacg

SEQ ID NO: 4 - DNA, 70 bp, synthetic construct
  source 1..70: mol_type = other DNA
ggagaattgtaatacgactcactatagggagacgcgtgatgtcaacaatgaggannnnnnnnnnnnnnnt

SEQ ID NO: 5 - DNA, 70 bp, synthetic construct
  source 1..70: mol_type = other DNA
ggagaattgtaatacgactcactatagggagacgcgtgccgtccacaatgaggannnnnnnnnnnnnnnt

SEQ ID NO: 6 - DNA, 70 bp, synthetic construct
  source 1..70: mol_type = other DNA
ggagaattgtaatacgactcactatagggagacgcgtggtagagacaatgaggannnnnnnnnnnnnnnt

SEQ ID NO: 7 - DNA, 70 bp, synthetic construct
  source 1..70: mol_type = other DNA
ggagaattgtaatacgactcactatagggagacgcgtggtgaaaacaatgaggannnnnnnnnnnnnnnt

SEQ ID NO: 8 - DNA, 70 bp, synthetic construct
  source 1..70: mol_type = other DNA
ggagaattgtaatacgactcactatagggagacgcgtggtggccacaatgaggannnnnnnnnnnnnnnt

SEQ ID NO: 9 - DNA, 70 bp, synthetic construct
  source 1..70: mol_type = other DNA
ggagaattgtaatacgactcactatagggagacgcgtggtttcgacaatgaggannnnnnnnnnnnnnnt

SEQ ID NO: 10 - DNA, 70 bp, synthetic construct
  source 1..70: mol_type = other DNA
ggagaattgtaatacgactcactatagggagacgcgtgcgtacgacaatgaggannnnnnnnnnnnnnnt

SEQ ID NO: 11 - DNA, 70 bp, synthetic construct
  source 1..70: mol_type = other DNA
ggagaattgtaatacgactcactatagggagacgcgtggagtggacaatgaggannnnnnnnnnnnnnnt

SEQ ID NO: 12 - DNA, 70 bp, synthetic construct
  source 1..70: mol_type = other DNA
ggagaattgtaatacgactcactatagggagacgcgtgggtagcacaatgaggannnnnnnnnnnnnnnt

SEQ ID NO: 13 - DNA, 18 bp, synthetic construct
  source 1..18: mol_type = other DNA
tagggagacgcgtgatca

SEQ ID NO: 14 - DNA, 17 bp, synthetic construct
  source 1..17: mol_type = other DNA
tagggagacgcgtgatc

SEQ ID NO: 15 - DNA, 16 bp, synthetic construct
  source 1..16: mol_type = other DNA
tagggagacgcgtgat

SEQ ID NO: 16 - DNA, 18 bp, synthetic construct
  source 1..18: mol_type = other DNA
tagggagacgcgtgagtt

SEQ ID NO: 17 - DNA, 18 bp, synthetic construct
  source 1..18: mol_type = other DNA
tagggagacgcgtgatgt

SEQ ID NO: 18 - DNA, 18 bp, synthetic construct
  source 1..18: mol_type = other DNA
tagggagacgcgtgccgt

SEQ ID NO: 19 - DNA, 18 bp, synthetic construct
  source 1..18: mol_type = other DNA
tagggagacgcgtggtag

SEQ ID NO: 20 - DNA, 18 bp, synthetic construct
  source 1..18: mol_type = other DNA
tagggagacgcgtggtga

SEQ ID NO: 21 - DNA, 18 bp, synthetic construct
  source 1..18: mol_type = other DNA
tagggagacgcgtggtgg

SEQ ID NO: 22 - DNA, 18 bp, synthetic construct
  source 1..18: mol_type = other DNA
tagggagacgcgtggttt

SEQ ID NO: 23 - DNA, 18 bp, synthetic construct
  source 1..18: mol_type = other DNA
tagggagacgcgtgcgta

SEQ ID NO: 24 - DNA, 18 bp, synthetic construct
  source 1..18: mol_type = other DNA
tagggagacgcgtggagt

SEQ ID NO: 25 - DNA, 18 bp, synthetic construct
  source 1..18: mol_type = other DNA
tagggagacgcgtgggta

SEQ ID NO: 26 - DNA, 18 bp, synthetic construct
  source 1..18: mol_type = other DNA
agacgcagaagagcagtg

SEQ ID NO: 27 - DNA, 91 bp, synthetic construct
  source 1..91: mol_type = other DNA
caaagagcagtctatctaggctggtgttttccacaaaatagggtgggtgggtgggttttctttcactcccttcaccatctatcctaatgtt

SEQ ID NO: 28 - DNA, 49 bp, synthetic construct
  source 1..49: mol_type = other DNA
gttttccacaaaatagggtgggtgggtgggttttctttcactcccttca

SEQ ID NO: 29 - DNA, 50 bp, synthetic construct
  source 1..50: mol_type = other DNA
aacattaggatagatggtgaaaaacaccagcctagatagactgctctttg

SEQ ID NO: 30 - DNA, 24 bp, synthetic construct
  source 1..24: mol_type = other DNA
aacattaggatagactgctctttg

SEQ ID NO: 31 - DNA, 44 bp, synthetic construct
  source 1..44: mol_type = other DNA
taataatggattgggtgggtgggtgggtacaaggatggagatga

SEQ ID NO: 32 - DNA, 47 bp, synthetic construct
  source 1..47: mol_type = other DNA
gtttgatggaaaaatgggtgggtgggtgggtagatgaatggatagat

SEQ ID NO: 33 - DNA, 21 bp, synthetic construct
  source 1..21: mol_type = other DNA
ccaatccattaaaattactct

SEQ ID NO: 34 - DNA, 21 bp, synthetic construct
  source 1..21: mol_type = other DNA
cccttttagacctaaaaggtg

SEQ ID NO: 35 - DNA, 60 bp, synthetic construct
  source 1..60: mol_type = other DNA
ggagaattgtaatacgactcactatagggagacgcgtgatcacgnnnnnnnnnnnnnnnt

SEQ ID NO: 36 - DNA, 62 bp, synthetic construct
  source 1..62: mol_type = other DNA
aaannnnnnnnnnnnnnncgtgatcacgcgtctccctatagtgagtcgtattacaattctcc

SEQ ID NO: 37 - DNA, 62 bp, synthetic construct
  source 1..62: mol_type = other DNA
cctcttaacattatgctgagtgatatccctctgcgcactagtgcnnnnnnnnnnnnnnnaaa